(19) United States
(12) Patent Application Publication
(10) Pub. No.: US 2019/0072541 A1
(43) Pub. Date: Mar. 7, 2019

(54) BIOMARKERS FOR TREATMENT OF ALOPECIA AREATA

(71) Applicant: THE TRUSTEES OF COLUMBIA UNIVERSITY IN THE CITY OF NEW YORK, New York, NY (US)

(72) Inventors: Angela Christiano, Mahwah, NJ (US); Raphael Clynes, West Nyack, NY (US)

(21) Appl. No.: 15/752,205

(22) PCT Filed: Aug. 15, 2016
(86) PCT No.: PCT/US16/47053

§ 371 Date: Feb. 12, 2018

Related U.S. Application Data
(60) Provisional application No. 62/205,476, filed on Aug. 14, 2015.

(51) Int. Cl. G01N 33/50; G01N 33/564

(52) U.S. Cl. CPC G01N 33/5023; G01N 33/564; G01N 2800/56; G01N 2800/52; G01N 2800/60

(57) ABSTRACT

The presently disclosed subject matter relates to biomarkers allowing for improved diagnosis and prognosis of alopecia areata as well as effective treatments for the disease, including methods that incorporate biomarkers capable of identifying patient sub-populations that will respond to such treatments and methods that incorporate biomarkers capable of tracking the progress of such treatments.

### CROSS-REFERENCE TO RELATED APPLICATIONS

This application claims priority to U.S. Provisional Patent Application Ser. No. 62/205,476, filed Aug. 14, 2015, which is hereby incorporated by reference in its entirety.

### GRANT INFORMATION

This invention was made with government support under NIH Grant Numbers R01AR056016, R21AR061881 and 5U01AR067173 awarded by the National Institutes of Health. The government has certain rights in the invention.

### 1. INTRODUCTION

The presently disclosed subject matter relates to biomarkers allowing for improved diagnosis and prognosis of Alopecia Areata as well as effective treatments for the disease, including methods that incorporate biomarkers capable of identifying patient sub-populations that will respond to such treatments and methods that incorporate biomarkers capable of tracking the progress of such treatments.

### 2. BACKGROUND

Alopecia areata (AA) is an autoimmune skin disease in which the hair follicle is the target of immune attack. Patients characteristically present with round or ovoid patches of hair loss usually on the scalp that can spontaneously resolve, persist, or progress to involve the scalp or the entire body. The three major phenotypic variants of the disease are patchy-type AA (AAP), which is often localized to small ovoid areas on the scalp or in the beard area, alopecia totalis (AT), which involves the entire scalp, and alopecia universalis (AU), which involves the entire body surface area.

There are currently no FDA approved drugs for AA, and treatment is often empiric but typically involves observation, intralesional steroids, topical immunotherapy or broad immunosuppressive treatments of unproven efficacy. The more severe forms of the disease, AU and AT, are often recalcitrant to treatment. Furthermore, a prevailing assumption among dermatologists and treating physicians is that long-standing AU and AT becomes irrecoverable, or transforms the scalp to a “burned out” state, supported by an inverse correlation between disease duration and responsiveness to treatment. Despite its high prevalence, there remains a need for biomarkers to identify the severity of the disease, as well as effective treatments for the disease, including methods that incorporate biomarkers capable of identifying patient sub-populations that will respond to such treatments and methods that incorporate biomarkers capable of tracking the progress of such treatments.

### 3. SUMMARY

The present disclosure relates to biomarkers allowing for improved diagnosis and prognosis of Alopecia Areata as well as effective treatments for the disease. In certain embodiments, a method of treating Alopecia Areata (AA) in a subject comprises identifying the AA disease severity in said subject by detecting a biomarker indicative of said disease severity, and administering a therapeutic intervention to said subject appropriate to the identified disease severity. The presently disclosed subject matter further provides a method of treating AA in a subject comprising identifying the propensity of a subject having AA to respond to JAK inhibitor treatment by detecting a biomarker indicative of said propensity, and administering a JAK inhibitor to said subject if the identified biomarker indicates a propensity that the subject will respond to said inhibitor. The presently disclosed subject matter further provides a method of treating Alopecia Areata (AA) in a subject comprising administering a JAK inhibitor to a subject having AA; and detecting a biomarker indicative of responsiveness to JAK inhibitor treatment.

In certain embodiments, said detection of the presently disclosed biomarker is performed on a sample obtained from the subject and the sample is selected from the group consisting of skin, blood, serum, plasma, urine, saliva, sputum, mucus, semen, amniotic fluid, mouth wash and bronchial lavage fluid. In certain embodiments, the subject is human. In certain embodiments, the sample is a skin sample. In certain embodiments, the sample is a serum sample. In certain embodiments, the biomarker is a gene expression signature. In certain embodiments, the gene expression signature comprises gene expression information of one or more of the following groups of genes: KRT-associated genes; CTL-associated genes; and IFN-associated genes. In certain embodiments, the KRT-associated genes comprise DSG4, HOXC31, KRT31, KRT32, KRT33B, KRT82, PKP1 and PKP2. In certain embodiments, the CTL-associated genes comprise CD8A, GZMB, ICOS and PRF1. In certain embodiments, the IFN-associated genes comprise CXCL9, CXCL10, CXCL11, STAT1 and MX1. In certain embodiments, the gene expression signature is an Alopecia Areata Disease Activity Index (ALADIN). In certain embodiments, the gene expression signature is an Alopecia Areata Gene Signature (AAGS) comprising one or more genes set forth in Table A. In certain embodiments, the gene expression signature is IKZF1, DLX4 or a combination thereof.

In certain embodiments, the detection of the presently disclosed biomarker is performed via a nucleic acid hybridization assay. In certain embodiments, the detection is performed via a microarray analysis. In certain embodiments, the detection is performed via polymerase chain reaction (PCR) or nucleic acid sequencing. In certain embodiments, the biomarker is a protein. In certain embodiments, the presence of the protein is detected using a reagent which specifically binds with the protein. In certain embodiments, the reagent is a monoclonal antibody or antigen-binding fragment thereof, or a polyclonal antibody or antigen-binding fragment thereof. In certain embodiments, the detection is performed via an enzyme-linked immunosorbent assay (ELISA), an immunofluorescence assay or a Western Blot assay.

In another aspect, the presently disclosed subject matter provides for a kit for treating Alopecia Areata (AA) in a subject comprising one or more detection reagents useful for detecting a biomarker indicative of a disease severity of the subject, and one or more treatment reagents useful for treating AA. The presently disclosed subject matter may further provide for a kit for treating Alopecia Areata (AA) in a subject comprising one or more detection reagents useful for detecting a biomarker indicative of a propensity of the subject to respond to one or more treatment reagent useful for treating AA, and one or more treatment reagents useful for treating AA. In certain embodiments, the kit further comprises one or more probe sets, arrays/microarrays, biomarker-specific antibodies and/or beads. In certain embodiments, the kit further comprises an instruction. In certain embodiments, the treatment reagent may be selected from a JAK inhibitor.

In certain embodiments, the JAK inhibitor is a compound that interacts with a Jak1/Jak2/Jak3/Tyk2/STAT1/STAT2/STAT3/STAT4/STAT5a/STAT5b/STAT6/OSM/gp 130/LIFR/OSM-Rβ gene or a Jak1/Jak2/Jak3/Tyk2/STAT1/STAT2/STAT3/STAT4/STAT5a/STAT5b/STAT6/OSM/gp130/LIFR/OSM-Rβ protein. In certain embodiments, the JAK inhibitor may be selected from ruxolitinib (INCB 018424), tofacitinib (CP690550), Tyrphostin AG490 (CAS Number: 133550-30-8), momelotinib (CYT387), pacritinib (SB1518), baricitinib (LY3009104), fedratinib (TG101348), BMS-911543 (CAS Number: 1271022-90-2), lestaurtinib (CEP-701), fludarabine, epigallocatechin-3-gallate (EGCG), peficitinib, ABT 494 (CAS Number: 1310726-60-3), AT 9283 (CAS Number: 896466-04-9), decernmotinib, filgotinib, gandotinib, INCB 39110 (CAS Number: 1334298-90-6), PF 04965842 (CAS Number: 1622902-68-4), R348 (R-932348, CAS Number: 916742-11-5; 1620142-65-5), AZD 1480 (CAS Number: 935666-88-9), cerdulatinib, INCB 052793, NS 018 (CAS Number: 1239358-86-1 (free base); 1239358-85-0 (HCl)), AC 410 (CAS Number: 1361415-84-0 (free base); 1361415-86-2 (HCl).), CT 1578 (SB 1578, CAS Number: 937273-04-6), JTE 052, PF 6263276, R 548, TG 02 (SB 1317, CAS Number: 937270-47-8), lumbricus rebellus extract, ARN 4079, AR 13154, UR 67767, CS510, VR588, DNX 04042, hyperforin, a derivative thereof, a deuterated variation thereof, a salt thereof, or a combination thereof. In certain embodiments, the detection reagent may be selected from a fluorescent reagent, a luminescent reagent, a dye, a radioisotope, a derivative thereof or a combination thereof.

### 4. BRIEF DESCRIPTION OF THE FIGURES

The Detailed Description, given by way of example but not intended to limit the invention to specific embodiments described, may be understood in conjunction with the accompanying drawings.

FIG. 1. Alopecia areata disease-specific signature. (A) Heat map of the 50 most differentially expressed genes with increased expression and 50 most differentially expressed genes with decreased expression within the AA-specific disease signature among AT/AU, AAP, and NC samples in the training set. (B) Expression terrain map of samples arrayed along the principal components of differential gene expression. The dots represent the location of each sample in the expression space (white=NC, blue=AAP, red=AT/AU), and the size of the peaks are generated based on the number of samples in the region (more juxtaposed samples produce higher, wider peaks). The principal component space can be condensed into a single numeric score reflecting the risk of a sample being a control, AAP, or AT/AU based on its location in the terrain space. This consensus score provides statistically significant separation control, AAP, and AT/AU sample cohorts (box-and-whiskers plot). Box denotes the interquartile range and median, whiskers denote the 5th and 95th percentiles, * indicates statistical significance against NC, † indicates statistical significance against AAP.

FIG. 2. Increased gene expression complexity and sustained inflammation in alopecia totalis and universalis. (A) Venn diagram of differentially expressed genes in AT/AU compared with normal (“AT/AU”) and AAP compared with normal (“AAP”). Shown are the numbers of differentially expressed genes within each section of the Venn diagram. (B) Perifollicular/peribulbar histopathological scores of CD3 infiltrates among skin sections from patients with AT/AU, AAP, or NC. * p<0.01; ** p<0.0005. (C) Representative histology images reflecting the HPS scores 0 (no infiltration) through 3 (severe infiltration). (D) List of KEGG pathways shared between AT/AU versus normal controls and AAP versus normal controls. (E) Network map of KEGG pathways upregulated in AT/AU versus normal controls (red), AAP versus normal controls (blue), or shared pathways in both AT/AU versus normal and AAP versus normal controls.

FIG. 3. Intraindividual gene expression analysis in AA. (A) Heat map comparing patient-matched lesional and nonlesional samples to identify genes that delineate them from each other as well as healthy controls. (B) Patient-matched lesional (red) and non-lesional (blue) samples arrayed by their normalized deviation from the first principal component of differential expression. Length of the line between paired samples indicates overall similarity (shorter lines) or dissimilarity (longer lines) based on the consensus of all signature genes. (C) A display using the first two principal components analysis of normal control samples, lesional AAP, and non-lesional AAP samples reveals that lesional samples cluster in between lesional samples and controls, rather than with either cohort. (D) Pathway and functional annotation analysis of the over- and under-expressed genes between lesional and non-lesional AAP samples reveals discrete sets of genes that are up in non-lesional and down in lesional (red nodes), and down in non-lesional and up in lesional (blue nodes). These genes link to the indicated enriched annotations (yellow nodes), representing functional molecular differences between lesional and non-lesional samples reflected in their expression profiles.

FIG. 4. Immune cell infiltrate gene expression signatures correlate with AA phenotype. (A), Relative estimates of the indicated infiltrating immune cells on a patient-by-patient basis based on consensus expression of corresponding immune markers (heatmap, right). Increasing red indicates increasing amounts of infiltrate. Patients are ranked by CD8 infiltration. Ranking patients by CD8 infiltration highest-to-lowest reveals population bias. The box-and-whiskers plot reflects the distribution of the indicated clinical presentations according to CD8 infiltration rank (lower rank indicates higher levels of infiltration). Box denotes the interquartile range and median, whiskers denote the 5th and 95th percentiles, * indicates statistical significance p=0.005, ** indicates p<lx10-5. (B) Using the consensus expression, infiltration contamination of the biopsy samples is estimated for each presentation cohort, AAP=patchy, (left pie) AT/AU=totalis/universalis (right pie), as well as the relative share of each immune tissue type in the total infiltration contaminant compared to unaffected controls (line chart). (C) Changes in estimated infiltration of each indicated immune type expressed as a fraction of total sample signal across NC, AAP, and AT/AU.

FIG. 5. ALADIN scores parallels disease phenotype. (A) Co-expression analysis of the genes differentially expressed between AA and healthy controls reveals 20 modules of genes. (B) GSEA of all 20 genes modules for enrichment in significant differential expression between AA and controls reveals that the green and brown modules are most highly enriched in comparisons. (C) Pathway analysis of these two modules (circles) reveals significant enrichment of several immune and immune response pathways (orange diamonds) and include genes previously implicated in GWAS (yellow), ALADIN CTL genes (magenta), CTL genes that are also GWAS hits (pink), and ALADIN IFN genes (turquoise). (D) The ALADIN score classifies patient samples in three dimensions integrating immune infiltration and structural changes reflected by gene expression to identify relative risk of AA severity in patients (Black: NC, Green: AAP, Red: AT/AU). (E) CTL (top panel), IFN (middle panel), and KRT (bottom panel) signature scores from patients with AU/AT with respect to disease duration.

FIG. 6. AA Validation Set. Dendrogram and heatmap of the 33 samples in the validation dataset. Hierarchical clustering using Euclidean distance and average linkage was performed using the 2002 Affymetrix PSIDs that were identified as differentially expressed between AA patients and normal controls in the Discovery dataset were used to cluster the samples.

FIG. 7A-7B. T cell immune gene signature among AA samples. (A) Unsupervised consensus clustering of AA patients and unaffected controls using signature genes unique to each infiltrating immune tissue allows for the relative quantification of infiltrates in each sample. In this heatmap, red indicates higher expression and white indicates lower expression. Three main superclusters are demarcated as Low, Medium (Med), and High relative levels of infiltration based on marker expression. (B) The three infiltration superclusters are statistically significantly correlated with prognosis. A 3×3 chi-squared test reveals that the severity of infiltration is predictive of the severity of the AA phenotype across these patients. The numbers displayed in each cell represents the percentage of each clinical presentation that is found in the accompanying supercluster, e.g., 72% of NC samples were found in the Low cluster. The chi-squared statistic and accompanying p-value are provided.

FIG. 8A-8C. Modules in AA disease specific signature define ALADIN components. (A) A dendrogram reflecting the gene co-expression clustering results. Along the bottom the colored barcode indicates the divisions that identified the 20 co-expressed modules used in this work. (B) A table of results when testing several clinical traits for association with the twenty modules. Displayed in each cell are the p-values for association between the corresponding module and trait. Cells are colored in increasing red to correspond to the significance of the association. (C) Gene Set Enrichment Analyses testing for statistical enrichment of each of the original ALADIN pathways in the AA cohort. In all comparisons against unaffected controls, there was statistical enrichment of the genes in the IFN, CTL, and KRT pathways in the direction expected (IFN and CTL are positively enriched, KRT is negatively enriched).

FIG. 9. ALADIN components differentiate AA phenotypes and normal controls. CTL (top panel), IFN (middle panel), and KRT (bottom panel) components of ALADIN were compared among normal control, AAP, and AU/AT samples. * p<0.05; ** p<0.0001.

FIG. 10. Duration does not significantly influence ALADIN component scores among AAP patients. CTL (top panel), IFN (middle panel), and KRT (bottom panel) components of ALADIN were compared among AAP patients with less than 5 years duration or at least 5 years duration. No significant differences were found.

FIG. 11A-11G. CD8+NKG2D+ cytotoxic T lymphocytes accumulate in the skin and are necessary and sufficient to induce disease in AA mice. (a) Immunofluorescence staining of NKG2D ligand (H60) in the hair follicle inner root sheath (marked by K71). Scale bar, 100 μm. (b) CD8+NKG2D+ cells in hair follicles of C57BL/6, healthy C3H/HeJ and C3H/HeJ AA mice. Top scale bar, 100 μm; bottom scale bar, 50 μm. (c) Cutaneous lymphadenopathy and hypercellularity in C3H/HeJ AA mice. (d) Frequency (number shown above boxed area) of CD8+NKG2D+ T cells in the skin and skin-draining lymph nodes in alopecic mice versus ungrafted mice. (e) Immunophenotype of CD8+NKG2D+ T cells in cutaneous lymph nodes of C3H/HeJ alopecic mice. (f) Left, Rae-1t expressing dermal sheath cells grown from C3H/HeJ hair follicles. Right, dose-dependent specific cell lysis induced by CD8+NKG2D+ T cells isolated from AA mice cutaneous lymph nodes in the presence of blocking anti-NKG2D antibody or isotype control. Effector to target ratio given as indicated. Data are expressed as means±s.d. (g) Hair loss in C3H/HeJ mice injected subcutaneously with total lymph node (LN) cells, CD8+NKG2D+ T cells alone, CD+NKG2D− T cells or lymph node cells depleted of NKG2D+(5 mice per group). Mice are representative of two experiments. ***P<0.001 (Fisher's exact test).

FIG. 12A-12I. Prevention of AA by blocking antibodies to IFN-γ, IL-2 or IL-15Rβ. C3H/HeJ grafted mice were treated systemically from the time of grafting. (a-h) AA development in C3H/HeJ grafted mice treated systemically from the time of grafting with antibodies to IFN-γ (a,b), IL-2 (d,e) and IL-15Rβ (g,h). Frequency (number shown above boxed area) of CD8+NKG2D+ T cells in the skin of mice treated with antibodies to IFN-γ (b), IL-2 (e) and IL-15Rβ (h) compared to PBS-treated mice. (*P<0.05, **P<0.01, ***P<0.001. Immunohistochemical staining of skin biopsies showing CD8 and MHC class I and II expression in skin of mice treated with isotype control antibody or with antibodies to IFN-γ (c), IL-2 (f) or IL-15Rβ (i). Scale bars, 100 μm.

FIG. 13A-13J. Systemic JAK1/2 or JAK3 inhibition prevents the onset of AA in grafted C3H/HeJ mice. (a j) AA development in C3H/HeJ grafted mice treated systemically from the time of grafting with ruxolitinib (JAK1/2i) (a,b) or tofacitinib (JAK3i) (f,g) (**P<0.01). Frequency (number shown above boxed area) of CD8+NKG2D+ T cells in skin and cutaneous lymph nodes of mice treated with PBS or with JAK1/2i (c) or JAK3i (h) (***P<0.001). Immunohistochemical staining of skin biopsies showing CD8 and MHC class I and II expression in skin of mice treated with PBS or with JAK1/2i (d) or JAK3i (i). ALADIN score of transcriptional analysis from mice treated with PBS or with JAK1/2i (e) or JAK3i (j), given as log 2 mean expression Z-scores. Hair regrowth after an additional 12 weeks after treatment withdrawal is also shown. (a,f). Scale bars, 100 μm.

FIG. 14A-14I. Reversal of established AA with topical small-molecule inhibitors of the downstream effector kinases JAK1/2 or JAK3, and clinical results of patients with AA. (a) Three mice per group with long-standing AA (at least 12 weeks after grafting) treated topically on the dorsal back with 0.5% JAK1/2i (center), 0.5% JAK3i (bottom) or vehicle alone (Aquaphor, top) by daily application for 12 weeks. This experiment was repeated three times. Hair regrowth at an additional 8 weeks after treatment withdrawal is also shown. (b) Time course of hair regrowth index shown as weeks after treatment. (c) The frequency (number shown above boxed area) of CD8+NKG2D+ T cells in the skin of mice treated with JAK1/2i or JAK3i compared to vehicle control mice (mean±s.e.m., n=3 per group, *P<0.05, **P<0.01). NS, not significant. (d) The ALADIN score shows treatment-related loss of CTL and IFN signatures, given as log 2 mean expression Z-scores. (e) Immunohistochemical staining of mouse skin biopsies shows treatment-related loss of expression of CD8 and MHC class I and II markers. Scale bar, 100 μm. (f) Treatment of patient 3 with AA, who had hair loss involving >80% of his scalp at baseline, with ruxolitinib and hair regrowth after 12 weeks of oral treatment. (g) Clinical correlative studies of biopsies obtained before treatment (baseline) and after 12 weeks of treatment of patient 2, including immunostains for CD4, CD8 and human leukocyte antigen (HLA) class I (A, B, C) and class II (DP, DQ, DR). Scale bar, 200 μm. (h,i) RNA microarray analysis from treated patients 1 and 2 with AA (before treatment versus after treatment versus 3 normal subjects) presented as a heatmap (h) and as a cumulative ALADIN index (i). KRT, hair follicle keratins.

FIG. 15. Clinical photographs of and serum CXCL10 and ALADIN profile of scalp skin biopsy samples from an AA patient treated with tofacitinib. Top panel, photographs were taken of the posterior scalp over 16 weeks of treatment with tofacitinib 5 mg twice daily. Bottom left panel, blood and scalp skin samples were taken at baseline and after 4 weeks of treatment with tofacitinib. CXCL10 ELISA was performed. Bottom middle panel, heat map of ALADIN genes from scalp skin samples taken from healthy control patients (normal) and the AA patient at baseline (TO) and after 4 weeks of treatment (T4). Bottom right panel, ALADIN plot of scalp skin samples taken from healthy control patients (black) and the AA patient at baseline (red) and after 4 weeks of treatment (yellow).

FIG. 16. Hair loss recurrence following cessation of oral tofacitinib treatment. Left panel, 8 weeks following cessation of treatment. Minimal hair loss could be appreciated. Right panel, 16 weeks following cessation of treatment. The patient exhibited almost complete loss of scalp hair.

FIG. 17. Scalp biopsy specimens from an alopecia areata patient treated with oral tofaicitinib. H&E stained scalp biopsy sections at basline (left panel) and following four weeks (right panel) of treatment with tofacitinib.

FIG. 18. Hair regrowth during and following discontinuation of ruxolitinib treatment. Top panel, SALT scores for individual patients during and following cessation of ruxolitinib treatment. Middle panel, percent regrowth for individual patients during and following cessation of ruxolitinib treatment. Bottom panel, predicted (black line) and actual patient regrowth trajectories (blue line) from regression models.

FIG. 19. Clinical photographs of responder AA patients on ruxolitinib. Left panels of each pair is at baseline, right panels are at the end of treatment with ruxolitinib.

FIG. 20. Biomarkers based on skin gene expression correlate with clinical response. A, Heat map and clustering dendrogram of samples from patients at baseline, week 12 of treatment, and healthy controls using differentially expressed genes between baseline responder and healthy control samples. B, Principal components plots of samples taken from subjects at 12 weeks post treatment and at baseline. C, Heat map of ALADIN genes. D, Three dimensional plot of ALADIN signatures. Black, normal subjects; red, AA responder patient at baseline; purple, AA patient after 12 weeks treatment; yellow, AA nonresponder patient at baseline; blue, AA non-responder patient after 12 weeks treatment. E, ALADIN component signature scores. Left panel, CTL signature scores; middle panel, IFN signature scores; right panel, KRT signature scores. * p<0.05, ** p<0.01, *** p<0.001.

FIG. 21: Clinical photographs of selected patients at baseline, end of treatment, and end of observation off treatment. A, D, G, Baseline photographs. B, E, H, End of treatment. C, F, I, End of observation off.

FIG. 22. ALADIN signatures normalize with treatment in responders. ALADIN component scores from skin samples of AA patients were determined at baseline, week 12, and, in certain cases, intermediate or post-treatment time points. Blue, responder patients; red, non-responder patients; black, normal control (NC) patients.

FIG. 23. Non-responder patients. A, C, E, Baseline photographs. B, D, F, End of treatment photographs.

FIG. 24A-24C. Gene Expression Analysis Identifies Mixed-Tissue Gene Signatures. (A) Unsupervised hierarchical clustering of a cohort of AAP, AT/AU, and unaffected controls (Normal) using the AAGS (blue, underexpression and red, overexpression). (B) Gene co-similarity matrix showing gene clusters. The stronger orange indicates lower dissimilarity in gene expression. The clusters over- and under-expressed in AA are indicated. (C) Graphical representation of genes in the signature and the statistically enriched functional categories associated with them. The blue indicates signaling pathways; the yellow indicates immune/inflammation pathways; the orange indicates HLA; and the red indicates cell death pathways. The pathways at p<0.05 FDR corrected were kept for this analysis.

FIG. 25A-25C. Identification of IKZF1 and DLX4 as MR. An overall flow of the pipeline used to deconvolve regulators of genes expressed in the end organ (skin) from those expressed in infiltrating tissue (immune cells). (A) Genes (aqua nodes labeled A-F) measured from a complex primary tissue sample are assigned to either end-organ (red, AAGS) or infiltrate (blue) based on whether or not they can be mapped to regulators in the skin network (R). Only the genes mapped to the red node are considered for MR analysis. The genes mapped to the blue node are pruned away. (B) The resulting pruning of the AAGS provides an end-organ-enriched gene expression signature (aqua nodes) that is mutually exclusive with an IGS, p=1.77×10−4, that is overexpressed (cluster 1, node sizes proportional to fold change) and suppressed (cluster 2) in AA. (C) To deconvolve the scalp skin regulators, MR analysis was performed on the AAGS and the IGS, yielding candidate regulators of each signature. The true MRs in the skin (R2) will only appear when using the AAGS and not in the IGS. The infiltrate regulators (R1) will not be detected using the AAGS. The IKZF1 and DLX4 only have significant FDR values when using the AAGS and are insignificant (FDR=1) when using the IGS, left. This analysis establishes IKZF1 and DLX4 as AAGS (aqua squares) and MRs (yellow squares) in the skin (right).

FIG. 26A-26E. Exongeous Expression of IKZF1 and DLX4 Induces a Context-Independent AA-like Gene Expression Signature. (A) 2D hierarchical clustering of gene expression measured in huDP and HK transfected with plasmid vectors expressing IKZF1, DLX4, or controls expressing RFP and IKZFδ, an isoform lacking DNA binding domains. The treatment type and cell type for each experiment are indicated at the top of the heatmap. The blue indicates decreased expression and the red indicates overexpression. (B) Analysis of IKZF1 and DLX4 mRNA expression in transfected cells in quadruplicate, represented as average ±SEM, normalized to B-actin. (C) Western blot confirming IKZF1 and DLX4 proteins. The GSEA plots measuring the specificity of AA-like response assayed by differential expression of the AAGS following (D) IKZF1 or (E) DLX4 overexpression. The genes are ranked left to right from most- to least-differentially expressed on the x axis and barcodes represent the positions of IKZF1 and for DLX4 signature genes. The Enrichment Score (ES) is shown in the plot, and the normalized Enrichment Score (nES) is displayed at the top. The nES is derived from the ES at the “leading edge” of the plot, that is, the first maximal ES peak obtained. The p value is computed for the nES compared against a randomized null distribution.

FIG. 27A-27C. Exogenous Expression of IKZF1 and DLX4 Induces Increased NKG2D-Dependent PBMC-Associated Cytotoxicity in Three Cultured Cell Types. The schematic on the left of each row describes the tissues introduced to PBMCs for cytotoxicity assays (in triplicate). The colors indicate host sources (matching colors indicate host-matched tissues). The middle bar graphs present the cytotoxicity values obtained after either 6 hr of incubation (total bar height) or the cytotoxicity observed after 6 hr with the addition of human anti-NKG2D monoclonal antibody (gray bar). The NKG2D-dependent cytotoxicity is the difference between the two (white bar). The right bar graphs report the changes in NKG2D-dependent cytotoxicity normalized to the RFP controls. IKZF1.2B indicates cells transfected with the IKZF1δ vector, and IKZF1.3B indicates the full-length transcript. The y axis reports cytotoxicity measured as a fraction of maximum cytotoxicity (total cell count). All error bars report ±SEM. ** indicate statistically significant difference from RFP control at FDR <0.05. (A) Dataseries corresponding to WB215J PBMCs and WB215J fibroblasts. (B) WB215J PBMCs against cultured huDP. (C) WB215J PBMCs against cultured HK.

FIG. 28A-28C. The Fully Reconstructed Master Regulator Module Predicts Both Immune Infiltration and Severity. (A) Using the exogenous expression data, it is possible to infer both direct transcriptional MR T (MR→T), as well as T regulated by TFs that are T of the MR (MR→TF→T). Any TF (TFB) that is paired with MRs IKZF1 or DLX4 (TFA) and that exhibits changes in expression upon overexpression of the TFA is regulated by the TFA. Subsequently, any genes (T) in the AAGS that are linked to TFB are secondary T of TFA (TFB responds). Any TFB that does not respond to transfection of TFA is not regulated by the TFA, so either TFB regulates TFA (TFB stable, left) or both are co-regulated by a third, TFC (TFB stable, right). (B) Using this approach, 78% of AAGS can be mapped to IKZF1 or DLX4 within one indirect TFB. The blue nodes represent AAGS genes that respond to IKZF1 or DLX4 expression, the size of nodes scaled to the fold change experimentally observed (only nodes having at least 25% change are shown). (C) Using these T, single numeric scores of IKZF1 and DLX4 transcriptional activity was generated and used to create classifiers for AA severity. The AA samples are then imposed over the search space to assess accuracy (top chart). The table provides quantitation and statistics for separation of presentations across territories in the search space (unaffected: NC; patchy AA: AAP; and totalis/universalis: AT/AU). The centroid representations can be used to show how populations transition into disease states by moving across the trained boundaries (bottom chart; nonlesional: AAP-N and lesional: AAP-L).

FIG. 29A-29B. Deconvolved Regulatory Modules Can Be Generated for AA, Ps, and AD Using the Same Naive Framework. (A) Disease-associated gene expression signatures for Ps and AD can be clearly defined by differential expression. The comparison of these signatures to the AA gene signature reveals that the AA signature is statistically distinct from both Ps and AD signatures (Fisher's exact test), whereas there is statistical evidence for some sharing between the Ps and AD signatures. (B) Translating these signatures into regulatory modules reveals entirely different MRs governing AD and Ps compared to AA. The yellow nodes=AA gene signature; the blue nodes=AD gene signature; the aqua nodes=Ps gene signature; the orange nodes=AA MR; the dark blue nodes=AD MR; and the cyan nodes=Ps MR. The list of top five AD and Ps MRs are provided, ranked by coverage of the corresponding signature. Also provided are the p values of each MR without deconvolution (IGS p value) (* indicates published regulators and † indicates an MR common to AD and Ps).

FIG. 30. Enriched pathways in the AAGS, FIG. 24. Supplemental Ingenuity Pathway Analysis shows enrichment of immune and cytotoxic signaling cascades for both infiltrating populations and end organ processes within the AAGS. Differentially expressed genes regulated by MRs include many membrane-bound, cell death- and Immune-associated proteins.

FIG. 31A-31B. AD and Ps disease gene signatures, FIG. 29. Unsupervised hierarchical clustering of lesional and unaffected patient samples using gene expression. Patients cleanly segregate by clinical presentation in both psoriasis (A) and atopic dermatitis (B) using the associated gene expression signatures. Sample dendrograms are provided here for reference for the heatmaps provided in FIG. 29. Psoriasis and Atopic Dermatitis cohorts have gene expression signatures that clearly delineate patients from unaffected controls

FIG. 32A-32B. Cytotoxicity assays, FIG. 27. Optimizations of PBMC concentration (A) and time window (B) for cytotoxicity assays identify a PBMC:target ratio of 100:1 and a time of at least 6 hours to achieve optimal separation.

FIG. 33 depicts a list of SNPs for use as biomarkers in connection with the instant disclosure.

FIG. 34 depicts a list of SNPs for use as biomarkers in connection with the instant disclosure.

FIG. 35 outlines the design of a clinical study of the treatment of AA by Ruxolitinib.

FIG. 36 outlines the status of the study described in FIG. 35.

FIG. 37 outlines the outcome of the study described in FIG. 35.

FIG. 38 depicts results obtained in connection with the study described in FIG. 35.

FIG. 39 depicts results obtained in connection with the study described in FIG. 35.

FIG. 40 depicts results obtained in connection with the study described in FIG. 35.

FIG. 41 depicts results obtained in connection with the study described in FIG. 35.

FIG. 42 depicts results obtained in connection with the study described in FIG. 35.

FIG. 43 depicts results obtained in connection with the study described in FIG. 35.

FIG. 44 depicts results obtained in connection with the study described in FIG. 35.

FIG. 45 outlines the design of a clinical study of the treatment of AA by Tofacitinib.

FIG. 46 depicts results obtained in connection with the study described in FIG. 45.

### 5. DETAILED DESCRIPTION

The presently disclosed subject matter relates to biomarkers allowing for improved diagnosis and prognosis of AA as well as effective treatments for the disease, including methods that incorporate biomarkers capable of identifying patient sub-populations that will respond to such treatments and methods that incorporate biomarkers capable of tracking the progress of such treatments.

### A. Definitions

According to the present disclosure, a “subject” or a “patient” is a human or non-human animal. Although the animal subject is preferably a human, the compounds and compositions of the invention have application in veterinary medicine as well, e.g., for the treatment of domesticated species such as canine, feline, murine, and various other pets; farm animal species such as bovine, equine, ovine, caprine, porcine, etc.; and wild animals, e.g., in the wild or in a zoological garden, such as non-human primates.

As used herein, the terms “treatment,” “treating,” and the like refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a subject or patient and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease

A “therapeutically effective amount” or “efficacious amount” refers to the amount of a compound or composition that, when administered to a mammal or other subject for treating a disease, is sufficient to effect such treatment for the disease. The “therapeutically effective amount” can vary depending on compound or composition used, the disease and its severity, and the age, weight, etc., of the subject to be treated.

The terms “pharmaceutical composition” and “pharmaceutical formulation,” as used herein, refer to a composition which is in such form as to permit the biological activity of an active ingredient contained therein to be effective, and which contains no additional components which are unacceptably toxic to a patient to which the formulation would be administered.

The term “pharmaceutically acceptable,” as used herein, e.g., with respect to a “pharmaceutically acceptable carrier,” refers to the property of being nontoxic to a subject. A pharmaceutically acceptable ingredient in a pharmaceutical formulation can be an ingredient other than an active ingredient which is nontoxic. A pharmaceutically acceptable carrier can include a buffer, excipient, stabilizer, and/or preservative.

As used herein, a “JAK inhibitor” refers to a compound that interacts with a Jak1/Jak2/Jak3/Tyk2/STAT1/STAT2/STAT3/STAT4/STAT5a/STAT5b/STAT6/OSM/gp 130/LIFR/OSM-Rβ gene or a Jak1/Jak2/Jak3/Tyk2/STAT1/STAT2/STAT3/STAT4/STAT5a/STAT5b/STAT6/OSM/gp130/LIFR/OSM-Rβ protein or polypeptide and inhibits its activity and/or its expression. The compound can decrease the activity or expression of a protein encoded by Jak1/Jak2/Jak3/Tyk2/STAT1/STAT2/STAT3/STAT4/STAT5 a/STAT5b/STAT6/0 SM/gp130/LIFR/OSM-Rβ. In certain embodiments, a JAK inhibitor can be a deuterated compound. In certain embodiments, the deuterated compound may be modified by deuteration at one or more sites on the compound.

A JAK inhibitor of the present disclosure can be a protein, such as an antibody (monoclonal, polyclonal, humanized, chimeric, or fully human), or a binding fragment thereof, directed against a polypeptide encoded by the corresponding sequence disclosed herein. An antibody fragment can be a form of an antibody other than the full-length form and includes portions or components that exist within full-length antibodies, in addition to antibody fragments that have been engineered, Antibody fragments can include, but are not limited to, single chain Fv (scFv), diabodies, Fv, and (Fab′) 2, triabodies, Fc, Fab, CDR1, CDR2, CDR3, combinations of CDR's, variable regions, tetrabodies, bifunctional hybrid antibodies, framework regions, constant regions, and the like. Antibodies can be obtained commercially, custom generated, or synthesized against an antigen of interest according to methods established in the art.

A JAK inhibitor of the present disclosure can be a small molecule that binds to a protein and disrupts its function. Small molecules are a diverse group of synthetic and natural substances generally having low molecular weights. They can be isolated from natural sources (for example, plants, fungi, microbes and the like), are obtained commercially and/or available as libraries or collections, or synthesized. Candidate small molecules that modulate a protein can be identified via in silico screening or high-through-put (HTP) screening of combinatorial libraries. Most conventional pharmaceuticals, such as aspirin, penicillin, and many chemotherapeutics, are small molecules, can be obtained commercially, can be chemically synthesized, or can be obtained from random or combinatorial libraries. In certain embodiments, the agent is a small molecule that binds, interacts, or associates with a target protein or RNA. Such a small molecule can be an organic molecule that, when the target is an intracellular target, is capable of penetrating the lipid bilayer of a cell to interact with the target. Small molecules include, but are not limited to, toxins, chelating agents, metals, and metalloid compounds. Small molecules can be attached or conjugated to a targeting agent so as to specifically guide the small molecule to a particular cell.

In certain embodiments, the JAK inhibitor is ruxolitinib (INCB 018424), tofacitinib (CP690550), Tyrphostin AG490 (CAS Number: 133550-30-8), momelotinib (CYT387), pacritinib (SB1518), baricitinib (LY3009104), fedratinib (TG101348), BMS-911543 (CAS Number: 1271022-90-2), lestaurtinib (CEP-701), fludarabine, epigallocatechin-3-gallate (EGCG), peficitinib, ABT 494 (CAS Number: 1310726-60-3), AT 9283 (CAS Number: 896466-04-9), decernmotinib, filgotinib, gandotinib, INCB 39110 (CAS Number: 1334298-90-6), PF 04965842 (CAS Number: 1622902-68-4), R348 (R-932348, CAS Number: 916742-11-5; 1620142-65-5), AZD 1480 (CAS Number: 935666-88-9), cerdulatinib, INCB 052793 (Incyte, clinical trial ID: NCT02265510), NS 018 (CAS Number: 1239358-86-1 (free base); 1239358-85-0 (HCl)), AC 410 (CAS Number: 1361415-84-0 (free base); 1361415-86-2 (HCl).), CT 1578 (SB 1578, CAS Number: 937273-04-6), JTE 052 (Japan Tobacco Inc.), PF 6263276 (Pfizer), R 548 (Rigel), TG 02 (SB 1317, CAS Number: 937270-47-8), lumbricus rebellus extract, ARN 4079 (Arrien Pharmaceuticals, LLC.), AR 13154 (Aerie Pharmaceuticals Inc.), UR 67767 (Palau Pharma S.A.), CS510 (Shenzhen Chipscreen Biosciences Ltd.), VR588 (Vectura Group plc), DNX 04042 (Dynamix Pharmaceuticals/Clevexel), hyperforin, or combinations thereof.

In certain embodiments, the JAK inhibitor is an antisense RNA, an siRNA, an shRNA, a microRNA, or a variant or modification thereof that specifically inhibits expression of the gene that encodes the Jak1, Jak2, Jak3, Tyk2, STAT1, STAT2, STAT3, STAT4, STAT5a, STAT5b, STATE, OSM, gp130, LIFR, or OSM-Rβ.

### B. Alopecia Areata Biomarkers

Embodiments of the present disclosure relate to methods of treating Alopecia Areata (AA) in a subject. In certain embodiments, a method for treating AA in a subject is disclosed, wherein the method includes: detecting a biomarker indicative of the disease severity and/or the propensity of the subject to respond to treatment before, during and/or after administering a therapeutic intervention to said subject. In certain embodiments, the biomarker is a gene expression signature. In certain embodiment, the gene expression signature comprises gene expression information of one or more of the following groups of genes: hair keratin (KRT) associated genes, cytotoxic T lymphocyte infiltration (CTL) associated genes, and interferon (IFN) associated genes. In certain embodiments, the KRT-associated genes comprise DSG4, HOXC31, KRT31, KRT32, KRT33B, KRT82, PKP1 and PKP2. In certain embodiments, the CTL-associated genes comprise CD8A, GZMB, ICOS and PRF1. In certain embodiments, the IFN-associated genes comprise CXCL9, CXCL10, CXCL11, STAT1 and MX1.

In certain embodiments, the gene expression signature is an Alopecia Areata Disease Activity Index (ALADIN). The Alopecia Areata Disease Activity Index (ALADIN) is a three-dimensional quantitative composite gene expression score for potential use as a biomarker for tracking disease severity and response to treatment. In certain embodiments, the ALADIN is based on gene expression of the CTL, IFN and KRT associated genes, wherein the CTL, IFN and KRT ALADIN scores are calculated for each sample of the subject. In certain embodiments, z-scores are calculated for each probe set relative to the mean and standard deviation of normal controls. Z-scores for each gene may be obtained by averaging z-scores of probe sets mapping to that gene. In certain embodiments, the signature scores are then calculated averages of the z-scores for genes belonging to the corresponding signature.

In certain embodiments, the biomarker is an Alopecia Areata Gene Signature (AAGS) comprising one or more genes set forth in Table A. In certain embodiments, the biomarker is IKZF1, DLX4 or a combination thereof

TABLE A

| | |
|---|---|
| NCBI | |
| GenBank | |
| ID | Official Gene Name |

T cell activation

| | |
|---|---|
| 596 | B-cell CLL/lymphoma 2 |
| 914 | CD2 molecule |
| 915 | CD3d molecule, delta (CD3-TCR complex) |
| 920 | CD4 molecule |
| 972 | CD74 molecule, major histocompatibility complex, class II |
| | invariant chain |
| 942 | CD86 molecule |
| 925 | CD8a molecule |
| 926 | CD8b molecule |
| 10320 | IKAROS family zinc finger 1 (lkaros) |
| 8440 | NCK adaptor protein 2 |
| 1499 | catenin (cadherin-associated protein), beta 1, 88 kDa |
| 55636 | chromodomain helicase DNA binding protein 7 |
| 8320 | eomesodermin homolog (Xenopus laevis) |
| 3683 | integrin, alpha L (antigen CD11A (p180), lymphocyte |
| | function-associated antigen 1; alpha polypeptide) |
| 3684 | integrin, alpha M (complement component 3 receptor 3 |
| | subunit) |
| 3659 | interferon regulatory factor 1 |
| 3600 | interleukin 15 |
| 3936 | lymphocyte cytosolic protein 1 (L-plastin) |
| 3932 | lymphocyte-specific protein tyrosine kinase |
| 3108 | major histocompatibility complex, class II, DM alpha |
| 6693 | sialophorin |
| 387357 | thymocyte_selection pathway associated |

immune response

| | |
|---|---|
| 596 | B-cell CLL/lymphoma 2 |
| 29760 | B-cell linker |
| 23601 | C-type lectin domain family 5, member A |
| 929 | CD14 molecule |
| 9332 | CD163 molecule |
| 100133941 | CD24 molecule; CD24 molecule-like 4 |
| 972 | CD74 molecule, major histocompatibility complex, class II |
| | invariant chain |
| 9308 | CD83 molecule |
| 8832 | CD84 molecule |
| 50848 | F11 receptor |
| 2268 | Gardner-Rasheed feline_sarcoma viral (v-fgr) oncogene |
| | homolog |
| 10866 | HLA complex P5 |
| 150372 | NFAT activating protein with ITAM motif 1 |
| 25939 | SAM domain and HD domain 1 |
| 50852 | T cell receptor associated transmembrane adaptor 1 |
| 7078 | TIMP metallopeptidase inhibitor 3 |
| 7305 | TYRO protein tyrosine kinase binding protein |
| 11326 | V-set and immunoglobulin domain containing 4 |
| 84632 | actin filament associated protein 1-like 2 |
| 90 | activin A receptor, type I |
| 199 | allograft inflammatory factor 1 |
| 197 | alpha-2-HS-glycoprotein |
| 60489 | apolipoprotein B mRNA editing enzyme, catalytic |
| | polypeptide-like 3G |
| 80833 | apolipoprotein L, 3 |
| 650 | bone morphogenetic protein 2 |
| 9435 | carbohydrate (N-acetylglucosamine-6-O)_sulfotransferase 2 |
| 1508 | cathepsin B |
| 6357 | chemokine (C-C motif) ligand 13 |
| 6362 | chemokine (C-C motif) ligand 18 (pulmonary and |
| | activation-regulated) |
| 6351 | chemokine (C-C motif) ligand 4 |
| 6352 | chemokine (C-C motif) ligand 5 |
| 6355 | chemokine (C-C motif) ligand 8 |
| 1230 | chemokine (C-C motif) receptor 1 |
| 1234 | chemokine (C-C motif) receptor 5 |
| 3627 | chemokine (C-X-C motif) ligand 10 |
| 4283 | chemokine (C-X-C motif) ligand 9 |
| 4261 | class II, major histocompatibility complex, transactivator |
| 713 | complement component 1, q_subcomponent, B chain |
| 714 | complement component 1, q_subcomponent, C chain |
| 1755 | deleted in malignant brain tumors 1 |
| 8456 | forkhead box N1 |
| 3055 | hemopoietic cell kinase |
| 8347, 8343, | histone cluster 1, H2bi; histone cluster 1, H2bg; histone |
| 8346, 8344, | cluster 1, H2be; histone cluster 1, H2bf; histone cluster 1, |
| 8339 | H2bc |
| 3399 | inhibitor of DNA binding 3, dominant negative helix-loop- |
| | helix protein |
| 3683 | integrin, alpha L (antigen CD11A (p180), lymphocyte |
| | function-associated antigen 1; alpha polypeptide) |
| 3689 | integrin, beta 2 (complement component 3 receptor 3 and 4 |
| | subunit) |
| 3694 | integrin, beta 6 |
| 64135 | interferon induced with helicase C domain 1 |
| 338376 | interferon, epsilon |
| 3600 | interleukin 15 |
| 9235 | interleukin 32 |
| 3579 | interleukin 8 receptor, beta |
| 3822 | killer cell lectin-like receptor_subfamily C, member 2 |
| 3988 | lipase A, lysosomal acid, cholesterol esterase |
| 58530 | lymphocyte antigen 6 complex, locus G6F; lymphocyte |
| | antigen 6 complex, locus G6D |
| 3107, 3106 | major histocompatibility complex, class I, C; major |
| | histocompatibility complex, class I, B |
| 4332 | myeloid cell nuclear differentiation antigen |
| 4542 | myosin IF |
| 5551 | perforin 1 (pore forming protein) |
| 30814 | phospholipase A2, group IIE |
| 5341 | pleckstrin |
| 10544 | protein C receptor, endothelial (EPCR) |
| 5265 | serpin peptidase inhibitor, clade A (alpha-1 antiproteinase, |
| | antitrypsin), member 1 |
| 12 | serpin peptidase inhibitor, clade A (alpha-1 antiproteinase, |
| | antitrypsin), member 3 |
| 6614 | sialic acid binding Ig-like lectin 1,_sialoadhesin |
| 6693 | sialophorin |
| 6469 | sonic hedgehog homolog (Drosophila) |
| 7057 | thrombospondin 1 |
| 7096 | toll-like receptor 1 |
| 7042 | transforming growth factor, beta 2 |
| 6890 | transporter 1, ATP-binding cassette,_sub-family B |
| | (MDR/TAP) |
| 7133 | tumor necrosis factor receptor_superfamily, member 1B |
| 7534 | tyrosine 3-monooxygenase/tryptophan 5-monooxygenase |
| | activation protein, zeta polypeptide |

plasma membrane

| | |
|---|---|
| 8909 | 26_serine protease |
| 417 | ADP-ribosyltransferase 1 |
| 24 | ATP-binding cassette,_sub-family A (ABC1), member 4 |
| 5243 | ATP-binding cassette,_sub-family B (MDR/TAP), member |
| | 1 |
| 5244 | ATP-binding cassette,_sub-family B (MDR/TAP), member |
| | 4 |
| 9619 | ATP-binding cassette,_sub-family G (WHITE), member 1 |
| 29760 | B-cell linker |
| 598 | BCL2-like 1 |
| 23601 | C-type lectin domain family 5, member A |
| 160365 | C-type lectin-like 1 |
| 135228 | CD109 molecule |
| 929 | CD14 molecule |
| 9332 | CD163 molecule |
| 911 | CD1c molecule |
| 914 | CD2 molecule |
| 30835 | CD209 molecule |
| 100133941 | CD24 molecule; CD24 molecule-like 4 |
| 915 | CD3d molecule, delta (CD3-TCR complex) |
| 920 | CD4 molecule |
| 1043 | CD52 molecule |
| 963 | CD53 molecule |
| 972 | CD74 molecule, major histocompatibility complex, class II |
| | invariant chain |
| 3732 | CD82 molecule |
| 9308 | CD83 molecule |
| 8832 | CD84 molecule |
| 942 | CD86 molecule |
| 925 | CD8a molecule |
| 926 | CD8b molecule |
| 10225 | CD96 molecule |
| 56882 | CDC42_small effector 1 |
| 30845 | EH-domain containing 3 |
| 1969 | EPH receptor A2 |
| 2048 | EPH receptor B2 |
| 50848 | F11 receptor |
| 22844 | FERM and PDZ domain containing 1 |
| 2205 | Fc fragment of IgE, high affinity I, receptor for; alpha |
| | polypeptide |
| 2212 | Fc fragment of IgG, low affinity IIa, receptor (CD32) |
| 2213 | Fc fragment of IgG, low affinity IIb, receptor (CD32); Fc |
| | fragment of IgG, low affinity IIc, receptor for (CD32) |
| 166647 | G protein-coupled receptor 125 |
| 23432 | G protein-coupled receptor 161 |
| 1880 | G protein-coupled receptor 183 |
| 55507 | G protein-coupled receptor, family C, group 5, member D |
| 3927 | LIM and SH3 protein 1 |
| 130576 | LY6/PLAUR domain containing 6B |
| 65108 | MARCKS-like 1 |
| 3071 | NCK-associated protein 1-like |
| 150372 | NFAT activating protein with ITAM motif 1 |
| 4864 | Niemann-Pick disease, type C1 |
| 5754 | PTK7 protein tyrosine kinase 7 |
| 376267 | RAB15, member RAS onocogene family |
| 22931 | RAB18, member RAS oncogene family |
| 285613 | RELT-like 2 |
| 56963 | RGM domain family, member A |
| 23504 | RIMS binding protein 2 |
| 10900 | RUN domain containing 3A |
| 6016 | Ras-like without CAAX 1 |
| 51458 | Rh family, C glycoprotein |
| 30011 | SH3-domain kinase binding protein 1 |
| 4092 | SMAD family member 7 |
| 8869 | ST3 beta-galactoside alpha-2,3-sialyltransferase 5 |
| 6461 | Src homology 2 domain containing adaptor protein B |
| 50852 | T cell receptor associated transmembrane adaptor 1 |
| 28639 | T cell receptor beta variable 19; T cell receptor beta |
| | constant 1 |
| 6967 | T cell receptor gamma locus; T cell receptor gamma |
| | constant 2 |
| 445347 | TCR gamma alternate reading frame protein; T cell |
| | receptor gamma variable 9; T cell receptor gamma |
| | constant 1 |
| 7305 | TYRO protein tyrosine kinase binding protein |
| 11326 | V-set and immunoglobulin domain containing 4 |
| 65266 | WNK lysine deficient protein kinase 4 |
| 10152 | abl interactor 2 |
| 90 | activin A receptor, type I |
| 120425 | adhesion molecule, interacts with CXADR antigen 1 |
| 199 | allograft inflammatory factor 1 |
| 83543 | allograft inflammatory factor 1-like |
| 351 | amyloid beta (A4) precursor protein |
| 56899 | ankyrin repeat and_sterile alpha motif domain containing |
| | 1B |
| 83464 | anterior pharynx defective 1 homolog B (C. elegans) |
| 54796 | basonuclin 2 |
| 144453 | bestrophin 3 |
| 685 | betacellulin |
| 1952 | cadherin, EGF LAG_seven-pass G-type receptor 2 |
| | (flamingo homolog, Drosophila) |
| 776 | calcium channel, voltage-dependent, L type, alpha 1D |
| | subunit |
| 8913 | calcium channel, voltage-dependent, T type, alpha 1G |
| | subunit |
| 27092 | calcium channel, voltage-dependent, gamma_subunit 4 |
| 800 | caldesmon 1 |
| 768 | carbonic anhydrase IX |
| 1499 | catenin (cadherin-associated protein), beta 1, 88 kDa |
| 1500 | catenin (cadherin-associated protein), delta 1 |
| 1501 | catenin (cadherin-associated protein), delta 2 (neural |
| | plakophilin-related arm-repeat protein) |
| 1508 | cathepsin B |
| 1230 | chemokine (C-C motif) receptor 1 |
| 1234 | chemokine (C-C motif) receptor 5 |
| 25932 | chloride intracellular channel 4 |
| 1464 | chondroitin_sulfate proteoglycan 4 |
| 23562 | claudin 14 |
| 1436 | colony_stimulating factor 1 receptor |
| 594855 | complexin 3 |
| 1525 | coxsackie virus and adenovirus receptor pseudogene 2; |
| | coxsackie virus and adenovirus receptor |
| 26999 | cytoplasmic FMR1 interacting protein 2 |
| 1824 | desmocollin 2 |
| 1830 | desmoglein 3 (pemphigus vulgaris antigen) |
| 147409 | desmoglein 4 |
| 55740 | enabled homolog (Drosophila) |
| 30816 | endogenous retroviral family W, env(C7), member 1 |
| 1946 | ephrin-A5 |
| 2099 | estrogen receptor 1 |
| 2260 | fibroblast growth factor receptor 1 |
| 23767 | fibronectin leucine rich transmembrane protein 3 |
| 54751 | filamin binding LIM protein 1 |
| 2323 | fms-related tyrosine kinase 3 ligand |
| 2350 | folate receptor 2 (fetal) |
| 342184 | formin 1 |
| 7976 | frizzled homolog 3 (Drosophila) |
| 8323 | frizzled homolog 6 (Drosophila) |
| 8324 | frizzled homolog 7 (Drosophila) |
| 2523 | fucosyltransferase 1 (galactoside 2-alpha-L- |
| | fucosyltransferase, H blood group) |
| 2554 | gamma-aminobutyric acid (GABA) A receptor, alpha 1 |
| 2561 | gamma-aminobutyric acid (GABA) A receptor, beta 2 |
| 2700 | gap junction protein, alpha 3, 46 kDa |
| 2706 | gap junction protein, beta 2, 26 kDa |
| 10804 | gap junction protein, beta 6, 30 kDa |
| 125111 | gap junction protein, delta 3, 31.9 kDa |
| 342035 | gliomedin |
| 2892 | glutamate receptor, ionotrophic, AMPA 3 |
| 3001 | granzyme A (granzyme 1, cytotoxic T-lymphocyte- |
| | associated_serine esterase 3) |
| 3002 | granzyme B (granzyme 2, cytotoxic T-lymphocyte- |
| | associated_serine esterase 1) |
| 2774 | guanine nucleotide binding protein (G protein), alpha |
| | activating activity polypeptide, olfactory type |
| 2782 | guanine nucleotide binding protein (G protein), beta |
| | polypeptide 1 |
| 115362 | guanylate binding protein 5 |
| 64399 | hedgehog interacting protein |
| 9456 | homer homolog 1 (Drosophila) |
| 9455 | homer homolog 2 (Drosophila) |
| 3683 | integrin, alpha L (antigen CD11A (p180), lymphocyte |
| | function-associated antigen 1; alpha polypeptide) |
| 3684 | integrin, alpha M (complement component 3 receptor 3 |
| | subunit) |
| 3687 | integrin, alpha X (complement component 3 receptor 4 |
| | subunit) |
| 3689 | integrin, beta 2 (complement component 3 receptor 3 and 4 |
| | subunit) |
| 3694 | integrin, beta 6 |
| 3587 | interleukin 10 receptor, alpha |
| 3594 | interleukin 12 receptor, beta 1 |
| 3600 | interleukin 15 |
| 3561 | interleukin 2 receptor, gamma (severe combined |
| | immunodeficiency) |
| 3579 | interleukin 8 receptor, beta |
| 182 | jagged 1 (Alagille_syndrome) |
| 58494 | junctional adhesion molecule 2 |
| 3821 | killer cell lectin-like receptor_subfamily C, member 1 |
| 3822 | killer cell lectin-like receptor_subfamily C, member 2 |
| 22914 | killer cell lectin-like receptor_subfamily K, member 1 |
| 8549 | leucine-rich repeat-containing G protein-coupled receptor 5 |
| 3977 | leukemia inhibitory factor receptor alpha |
| 58530 | lymphocyte antigen 6 complex, locus G6F; lymphocyte |
| | antigen 6 complex, locus G6D |
| 3936 | lymphocyte cytosolic protein 1 (L-plastin) |
| 3932 | lymphocyte-specific protein tyrosine kinase |
| 4033 | lymphoid-restricted membrane protein |
| 9170 | lysophosphatidic acid receptor 2 |
| 23566 | lysophosphatidic acid receptor 3 |
| 3107, 3106 | major histocompatibility complex, class I, C; major |
| | histocompatibility complex, class I, B |
| 3134 | major histocompatibility complex, class I, F |
| 3108 | major histocompatibility complex, class II, DM alpha |
| 3109 | major histocompatibility complex, class II, DM beta |
| 3111 | major histocompatibility complex, class II, DO alpha |
| 3113 | major histocompatibility complex, class II, DP alpha 1 |
| 3119 | major histocompatibility complex, class II, DQ beta 1; |
| | similar to major histocompatibility complex, class II, |
| | DQ beta 1 |
| 4118 | mal, T-cell differentiation protein |
| 4360 | mannose receptor, C type 1 |
| 55686 | melanoregulin |
| 154043, | membrane associated guanylate kinase, WW and PDZ |
| 9223 | domain containing 1; CNKSR family member 3 |
| 23499 | microtubule-actin crosslinking factor 1 |
| 9053 | microtubule-associated protein 7 |
| 4128 | monoamine oxidase A |
| 4155 | myelin basic protein |
| 8828 | neuropilin 2 |
| 4846 | nitric oxide_synthase 3 (endothelial cell) |
| 123264 | organic_solute transporter beta |
| 29780 | parvin, beta |
| 64098 | parvin, gamma |
| 5551 | perforin 1 (pore forming protein) |
| 5141 | phosphodiesterase 4A, cAMP-specific (phosphodiesterase |
| | E2 dunce homolog, Drosophila) |
| 27445 | piccolo (presynaptic cytomatrix protein) |
| 5317 | plakophilin 1 (ectodermal dysplasia/skin fragility |
| | syndrome);_similar to plakophilin 1 isoform 1a |
| 11187 | plakophilin 3 |
| 5341 | pleckstrin |
| 23362 | pleckstrin and Sec7 domain containing 3 |
| 5362 | plexin A2 |
| 5818 | poliovirus receptor-related 1 (herpesvirus entry mediator C) |
| 81607 | poliovirus receptor-related 4 |
| 200845 | potassium channel tetramerisation domain containing 6 |
| 3784 | potassium voltage-gated channel, KQT-like_subfamily, |
| | member 1 |
| 56937 | prostate transmembrane protein, androgen induced 1 |
| 10544 | protein C receptor, endothelial (EPCR) |
| 5579 | protein kinase C, beta |
| 5587 | protein kinase D1 |
| 26051 | protein phosphatase 1, regulatory (inhibitor)_subunit 16B |
| 5099 | protocadherin 7 |
| 53829 | purinergic receptor P2Y, G-protein coupled, 13 |
| 9934 | purinergic receptor P2Y, G-protein coupled, 14 |
| 54509 | ras homolog gene family, member F (in filopodia) |
| 9699 | regulating_synaptic membrane exocytosis 2 |
| 9783 | regulating_synaptic membrane exocytosis 3 |
| 6248 | regulatory_solute carrier protein, family 1, member 1 |
| 22800 | related RAS viral (r-ras) oncogene homolog 2;_similar to |
| | related RAS viral (r-ras) oncogene homolog 2 |
| 6404 | selectin P ligand |
| 64218 | sema domain, immunoglobulin domain (Ig), |
| | transmembrane domain (TM) and_short cytoplasmic |
| | domain, (semaphorin) 4A |
| 6614 | sialic acid binding Ig-like lectin 1,_sialoadhesin |
| 89790 | sialic acid binding Ig-like lectin 10 |
| 6693 | sialophorin |
| 140885 | signal-regulatory protein alpha |
| 55423 | signal-regulatory protein gamma |
| 6504 | signaling lymphocytic activation molecule family member |
| | 1 |
| 4301 | similar to Afadin (Protein AF-6); myeloid/lymphoid or |
| | mixed-lineage leukemia (trithorax homolog, Drosophila); |
| | translocated to, 4 |
| 57228 | small trans-membrane and glycosylated protein |
| 6509 | solute carrier family 1 (glutamate/neutral amino acid |
| | transporter), member 4 |
| 6511 | solute carrier family 1 (high affinity aspartate/glutamate |
| | transporter), member 6 |
| 10723 | solute carrier family 12 (potassium/chloride transporters), |
| | member 7 |
| 9120 | solute carrier family 16, member 6 (monocarboxylic acid |
| | transporter 7);_similar to_solute carrier family 16, member 6 |
| 220963 | solute carrier family 16, member 9 (monocarboxylic acid |
| | transporter 9) |
| 6575 | solute carrier family 20 (phosphate transporter), member 2 |
| 28965 | solute carrier family 27 (fatty acid transporter), member 6 |
| 10991 | solute carrier family 38, member 3 |
| 30061 | solute carrier family 40 (iron-regulated transporter), |
| | member 1 |
| 200010 | solute carrier family 5 (sodium/glucose cotransporter), |
| | member 9 |
| 11254 | solute carrier family 6 (amino acid transporter), member 14 |
| 55117 | solute carrier family 6 (neutral amino acid transporter), |
| | member 15 |
| 23428 | solute carrier family 7 (cationic amino acid transporter, y+ |
| | system), member 8 |
| 23657 | solute carrier family 7, (cationic amino acid transporter, y+ |
| | system) member 11 |
| 6751 | somatostatin receptor 1 |
| 6752 | somatostatin receptor 2 |
| 6469 | sonic hedgehog homolog (Drosophila) |
| 6272 | sortilin 1 |
| 124460 | sorting nexin 20 |
| 2040 | stomatin |
| 6854 | synapsin 11 |
| 54843 | synaptotagmin-like 2 |
| 117178 | synovial_sarcoma, X breakpoint 2 interacting protein |
| 7057 | thrombospondin 1 |
| 6915 | thromboxane A2 receptor |
| 7096 | toll-like receptor 1 |
| 7039 | transforming growth factor, alpha |
| 7053 | transglutaminase 3 (E polypeptide, protein-glutamine- |
| | gamma-glutamyltransferase) |
| 140803 | transient receptor potential cation channel,_subfamily M, |
| | member 6 |
| 4071 | transmembrane 4 L_six family member 1 |
| 6890 | transporter 1, ATP-binding cassette,_sub-family B |
| | (MDR/TAP) |
| 10381 | tubulin, beta 3; melanocortin 1 receptor (alpha melanocyte |
| | stimulating hormone receptor) |
| 8795 | tumor necrosis factor receptor_superfamily, member 10b |
| 51330 | tumor necrosis factor receptor_superfamily, member 12A |
| 7133 | tumor necrosis factor receptor_superfamily, member 1B |
| 7126 | tumor necrosis factor, alpha-induced protein 1 (endothelial) |
| 94015 | tweety homolog 2 (Drosophila) |
| 5412 | ubiquitin-like 3 |
| 673 | v-raf murine_sarcoma viral oncogene homolog B1 |
| 6843 | vesicle-associated membrane protein 1 (synaptobrevin 1) |

antigen presentation

| | |
|---|---|
| 920 | CD4 molecule |
| 972 | CD74 molecule, major histocompatibility complex, class II |
| | invariant chain |
| 925 | CD8a molecule |
| 926 | CD8b molecule |
| 1508 | cathepsin B |
| 1520 | cathepsin S |
| 4261 | class II, major histocompatibility complex, transactivator |
| 3306 | heat_shock 70 kDa protein 2 |
| 3821 | killer cell lectin-like receptor_subfamily C, member 1 |
| 3822 | killer cell lectin-like receptor_subfamily C, member 2 |
| 3107, 3106 | major histocompatibility complex, class I, C; major |
| | histocompatibility complex, class I, B |
| 3134 | major histocompatibility complex, class I, F |
| 3108 | major histocompatibility complex, class II, DM alpha |
| 3109 | major histocompatibility complex, class II, DM beta |
| 3111 | major histocompatibility complex, class II, DO alpha |
| 3113 | major histocompatibility complex, class II, DP alpha 1 |
| 3119 | major histocompatibility complex, class II, DQ beta 1; |
| | similar to major histocompatibility complex, class II, DQ |
| | beta 1 |
| 2923 | protein disulfide isomerase family A, member 3 |
| 5993 | regulatory factor X, 5 (influences HLA class II expression) |
| 6890 | transporter 1, ATP-binding cassette,_sub-family B |
| | (MDR/TAP) |

hair cycle/hair follicle dev/epidermis dev

| | |
|---|---|
| 596 | B-cell CLL/lymphoma 2 |
| 8538 | BARX homeobox 2 |
| 2001 | E74-like factor 5 (ets domain transcription factor) |
| 646 | basonuclin 1 |
| 1499 | catenin (cadherin-associated protein), beta 1, 88 kDa |
| 1474 | cystatin ELM |
| 2068 | excision repair cross-complementing rodent repair |
| | deficiency, complementation group 2 |
| 2171 | fatty acid binding protein 5-like 2; fatty acid binding |
| | protein 5 (psoriasis-associated); fatty acid binding protein |
| | 5-like 8; fatty acid binding protein 5-like 7; fatty acid |
| | binding protein 5-like 9 |
| 2304 | forkhead box E1 (thyroid transcription factor 2) |
| 8456 | forkhead box N1 |
| 3229 | homeobox C13 |
| 182 | jagged 1 (Alagille_syndrome) |
| 3868 | keratin 16; keratin type 16-like |
| 342574 | keratin 27 |
| 3881 | keratin 31 |
| 3882 | keratin 32 |
| 3885 | keratin 34 |
| 3854 | keratin 6B |
| 3889 | keratin 83 |
| 3891 | keratin 85 |
| 3846 | keratin associated protein 5-9 |
| 51176 | lymphoid enhancer-binding factor 1 |
| 55686 | melanoregulin |
| 5017 | ovo-like 1(Drosophila) |
| 864 | runt-related transcription factor 3 |
| 6469 | sonic hedgehog homolog (Drosophila) |
| 7042 | transforming growth factor, beta 2 |
| 7053 | transglutaminase 3 (E polypeptide, protein-glutamine- |
| | gamma-glutamyltransferase) |

### C. Biomarker Detection

A biomarker used in the methods of this disclosure can be identified in a biological sample using any method known in the art. Determining the presence of a biomarker, protein or degradation product thereof, the presence of mRNA or pre-mRNA, or the presence of any biological molecule or product that is indicative of biomarker expression, or degradation product thereof, can be carried out for use in the methods of the disclosure by any method described herein or known in the art.

Nucleic Acid Detection Techniques

Any method for qualitatively or quantitatively detecting a nucleic acid biomarker can be used. For example, detection of RNA transcripts can be achieved, for example, by Northern blotting, wherein a preparation of RNA is run on a denaturing agarose gel, and transferred to a suitable support, such as activated cellulose, nitrocellulose or glass or nylon membranes. Radiolabeled cDNA or RNA is then hybridized to the preparation, washed and analyzed by autoradiography.

Detection of RNA transcripts can further be accomplished using amplification methods. For example, it is within the scope of the present disclosure to reverse transcribe mRNA into cDNA followed by polymerase chain reaction (RT-PCR); or, to use a single enzyme for both steps as described in U.S. Pat. No. 5,322,770, or reverse transcribe mRNA into cDNA followed by symmetric gap ligase chain reaction (RT-AGLCR) as described by R. L. Marshall, et al., PCR Methods and Applications 4: 80-84 (1994).

In certain embodiments, quantitative real-time polymerase chain reaction (qRT-PCR) is used to evaluate mRNA levels of biomarker. The levels of a biomarker and a control mRNA can be quantitated in affected tissues or cells and adjacent unaffected tissues. In one specific embodiment, the levels of one or more biomarkers can be quantitated in a biological sample.

Other known amplification methods which can be utilized herein include but are not limited to the so-called “NASBA” or “3SR” technique described in PNAS USA 87: 1874-1878 (1990) and also described in Nature 350 (No. 6313): 91-92 (1991); Q-beta amplification as described in published European Patent Application (EPA) No. 4544610; strand displacement amplification (as described in G. T. Walker et al., Clin. Chem. 42: 9-13 (1996) and European Patent Application No. 684315; and target mediated amplification, as described by PCT Publication WO9322461.

In situ hybridization visualization can also be employed, wherein a radioactively labeled antisense RNA probe is hybridized with a thin section of a biopsy sample, washed, cleaved with RNase and exposed to a sensitive emulsion for autoradiography. The samples can be stained with haematoxylin to demonstrate the histological composition of the sample, and dark field imaging with a suitable light filter shows the developed emulsion. Non-radioactive labels such as digoxigenin can also be used.

Another method for evaluation of biomarker expression is to detect mRNA levels of a biomarker by fluorescent in situ hybridization (FISH). FISH is a technique that can directly identify a specific region of DNA or RNA in a cell and therefore enables to visual determination of the biomarker expression in tissue samples. The FISH method has the advantages of a more objective scoring system and the presence of a built-in internal control consisting of the biomarker gene signals present in all non-neoplastic cells in the same sample. Fluorescence in situ hybridization is a direct in situ technique that is relatively rapid and sensitive. FISH test also can be automated. Immunohistochemistry can be combined with a FISH method when the expression level of the biomarker is difficult to determine by immunohistochemistry alone.

Alternatively, mRNA expression can be detected on a DNA array, chip or a microarray. Oligonucleotides corresponding to the biomarker(s) are immobilized on a chip which is then hybridized with labeled nucleic acids of a test sample obtained from a subject. Positive hybridization signal is obtained with the sample containing biomarker transcripts. Methods of preparing DNA arrays and their use are well known in the art. (See, for example, U.S. Pat. Nos. 6,618,6796; 6,379,897; 6,664,377; 6,451,536; 548,257; U.S. 20030157485 and Schena et al. 1995 Science 20:467-470; Gerhold et al. 1999 Trends in Biochem. Sci. 24, 168-173; and Lennon et al. 2000 Drug discovery Today 5: 59-65, which are herein incorporated by reference in their entirety). Serial Analysis of Gene Expression (SAGE) can also be performed (See, for example, U.S. Patent Application 20030215858).

To monitor mRNA levels, for example, mRNA can be extracted from the biological sample to be tested, reverse transcribed, and fluorescent-labeled cDNA probes are generated. The microarrays are capable of hybridizing to a biomarker. cDNA can then probed with the labeled cDNA probes, the slides scanned and fluorescence intensity measured. This intensity correlates with the hybridization intensity and expression levels.

Types of probes for detection of RNA include cDNA, riboprobes, synthetic oligonucleotides and genomic probes. The type of probe used will generally be dictated by the particular situation, such as riboprobes for in situ hybridization, and cDNA for Northern blotting, for example. In certain embodiments, the probe is directed to nucleotide regions unique to the particular biomarker RNA. The probes can be as short as is required to differentially recognize the particular biomarker mRNA transcripts, and can be as short as, for example, 15 bases; however, probes of at least 17 bases, at least 18 bases and at least 20 bases can be used. In certain embodiments, the primers and probes hybridize specifically under stringent conditions to a nucleic acid fragment having the nucleotide sequence corresponding to the target gene. As herein used, the term “stringent conditions” means hybridization will occur only if there is at least 95% or at least 97% identity between the sequences.

The form of labeling of the probes can be any that is appropriate, such as the use of radioisotopes, for example, ³²P and ³⁵S. Labeling with radioisotopes can be achieved, whether the probe is synthesized chemically or biologically, by the use of suitably labeled bases.

Protein Detection Techniques

Methods for the detection of protein biomarkers are well known to those skilled in the art, and include but are not limited to mass spectrometry techniques, 1-D or 2-D gel-based analysis systems, chromatography, enzyme linked immunosorbent assays (ELISAs), radioimmunoassays (RIA), enzyme immunoassays (EIA), Western Blotting, immunoprecipitation and immunohistochemistry. These methods use antibodies, or antibody equivalents, to detect protein, or use biophysical techniques. Antibody arrays or protein chips can also be employed, see for example U.S. Patent Application Nos: 2003/0013208A1; 2002/0155493A1, 2003/0017515 and U.S. Pat. Nos. 6,329,209 and 6,365,418, herein incorporated by reference in their entireties.

ELISA and MA procedures can be conducted such that a biomarker standard is labeled (with a radioisotope such as ¹²⁵I or ³⁵S, or an assayable enzyme, such as horseradish peroxidase or alkaline phosphatase), and, together with the unlabeled sample, brought into contact with the corresponding antibody, whereon a second antibody is used to bind the first, and radioactivity or the immobilized enzyme assayed (competitive assay). Alternatively, the biomarker in the sample is allowed to react with the corresponding immobilized antibody, radioisotope or enzyme-labeled anti-biomarker antibody is allowed to react with the system, and radioactivity or the enzyme assayed (ELISA-sandwich assay). Other conventional methods can also be employed as suitable.

The above techniques can be conducted essentially as a “one-step” or “two-step” assay. A “one-step” assay involves contacting antigen with immobilized antibody and, without washing, contacting the mixture with labeled antibody. A “two-step” assay involves washing before contacting the mixture with labeled antibody. Other conventional methods can also be employed as suitable.

In certain embodiments, a method for measuring biomarker expression includes the steps of: contacting a biological sample, e.g., blood and/or plasma, with an antibody or variant (e.g., fragment) thereof which selectively binds the biomarker, and detecting whether the antibody or variant thereof is bound to the sample. A method can further include contacting the sample with a second antibody, e.g., a labeled antibody. The method can further include one or more steps of washing, e.g., to remove one or more reagents.

It can be desirable to immobilize one component of the assay system on a support, thereby allowing other components of the system to be brought into contact with the component and readily removed without laborious and time-consuming labor. It is possible for a second phase to be immobilized away from the first, but one phase is usually sufficient.

It is possible to immobilize the enzyme itself on a support, but if solid-phase enzyme is required, then this is generally best achieved by binding to antibody and affixing the antibody to a support, models and systems for which are well-known in the art. Simple polyethylene can provide a suitable support.

Enzymes employable for labeling are not particularly limited, but can be selected, for example, from the members of the oxidase group. These catalyze production of hydrogen peroxide by reaction with their substrates, and glucose oxidase is often used for its good stability, ease of availability and cheapness, as well as the ready availability of its substrate (glucose). Activity of the oxidase can be assayed by measuring the concentration of hydrogen peroxide formed after reaction of the enzyme-labeled antibody with the substrate under controlled conditions well-known in the art.

Other techniques can be used to detect a biomarker according to a practitioner's preference based upon the present disclosure. One such technique that can be used for detecting and quantitating biomarker protein levels is Western blotting (Towbin et al., Proc. Nat. Acad. Sci. 76:4350 (1979)). Cells can be frozen, homogenized in lysis buffer, and the lysates subjected to SDS-PAGE and blotting to a membrane, such as a nitrocellulose filter. Antibodies (unlabeled) are then brought into contact with the membrane and assayed by a secondary immunological reagent, such as labeled protein A or anti-immunoglobulin (suitable labels including ¹²⁵I, horseradish peroxidase and alkaline phosphatase). Chromatographic detection can also be used. In certain embodiments, immunodetection can be performed with antibody to a biomarker using the enhanced chemiluminescence system (e.g., from PerkinElmer Life Sciences, Boston, Mass.). The membrane can then be stripped and re-blotted with a control antibody, e.g., anti-actin (A-2066) polyclonal antibody from Sigma (St. Louis, Mo.).

Immunohistochemistry can be used to detect the expression and/presence of a biomarker, e.g., in a biopsy sample. A suitable antibody is brought into contact with, for example, a thin layer of cells, followed by washing to remove unbound antibody, and then contacted with a second, labeled, antibody. Labeling can be by fluorescent markers, enzymes, such as peroxidase, avidin or radiolabeling. The assay is scored visually, using microscopy and the results can be quantitated.

Other machine or autoimaging systems can also be used to measure immunostaining results for the biomarker. As used herein, “quantitative” immunohistochemistry refers to an automated method of scanning and scoring samples that have undergone immunohistochemistry, to identify and quantitate the presence of a specified biomarker, such as an antigen or other protein. The score given to the sample is a numerical representation of the intensity of the immunohistochemical staining of the sample, and represents the amount of target biomarker present in the sample. As used herein, Optical Density (OD) is a numerical score that represents intensity of staining. As used herein, semi-quantitative immunohistochemistry refers to scoring of immunohistochemical results by human eye, where a trained operator ranks results numerically (e.g., as 1, 2 or 3).

Various automated sample processing, scanning and analysis systems suitable for use with immunohistochemistry are available in the art. Such systems can include automated staining (see, e.g., the Benchmark system, Ventana Medical Systems, Inc.) and microscopic scanning, computerized image analysis, serial section comparison (to control for variation in the orientation and size of a sample), digital report generation, and archiving and tracking of samples (such as slides on which tissue sections are placed). Cellular imaging systems are commercially available that combine conventional light microscopes with digital image processing systems to perform quantitative analysis on cells and tissues, including immunostained samples. See, e.g., the CAS-200 system (Becton, Dickinson & Co.).

Antibodies against biomarkers can also be used for imaging purposes, for example, to detect the presence of a biomarker in cells of a subject. Suitable labels include radioisotopes, iodine (¹²⁵I, ¹²¹J) carbon (¹⁴C), sulphur (³⁵S), tritium (³H), indium (¹¹²In), and technetium (^{99m}Tc), fluorescent labels, such as fluorescein and rhodamine and biotin. Immunoenzymatic interactions can be visualized using different enzymes such as peroxidase, alkaline phosphatase, or different chromogens such as DAB, AEC or Fast Red.

Antibodies and derivatives thereof that can be used encompasses polyclonal or monoclonal antibodies, chimeric, human, humanized, primatized (CDR-grafted), veneered or single-chain antibodies, phase produced antibodies (e.g., from phage display libraries), as well as functional binding fragments, of antibodies. For example, antibody fragments capable of binding to a biomarker, or portions thereof, including, but not limited to Fv, Fab, Fab′ and F(ab′)₂ fragments can be used. Such fragments can be produced by enzymatic cleavage or by recombinant techniques. For example, papain or pepsin cleavage can generate Fab or F(ab′)₂ fragments, respectively. Other proteases with the requisite substrate specificity can also be used to generate Fab or F(ab′)₂ fragments. Antibodies can also be produced in a variety of truncated forms using antibody genes in which one or more stop codons have been introduced upstream of the natural stop site. For example, a chimeric gene encoding a F(ab′)₂ heavy chain portion can be designed to include DNA sequences encoding the CH, domain and hinge region of the heavy chain.

Synthetic and engineered antibodies are described in, e.g., Cabilly et al., U.S. Pat. No. 4,816,567 Cabilly et al., European Patent No. 0,125,023 B1; Boss et al., U.S. Pat. No. 4,816,397; Boss et al., European Patent No. 0,120,694 B1; Neuberger, M. S. et al., WO 86/01533; Neuberger, M. S. et al., European Patent No. 0,194,276 B1; Winter, U.S. Pat. No. 5,225,539; Winter, European Patent No. 0,239,400 B1; Queen et al., European Patent No. 0451216 B1; and Padlan, E. A. et al., EP 0519596 A1. See also, Newman, R. et al., BioTechnology, 10: 1455-1460 (1992), regarding primatized antibody, and Ladner et al., U.S. Pat. No. 4,946,778 and Bird, R. E. et al., Science, 242: 423-426 (1988)) regarding single-chain antibodies.

In certain embodiments, agents that specifically bind to a polypeptide other than antibodies are used, such as peptides. Peptides that specifically bind can be identified by any means known in the art, e.g., peptide phage display libraries. Generally, an agent that is capable of detecting a biomarker polypeptide, such that the presence of a biomarker is detected and/or quantitated, can be used. As defined herein, an “agent” refers to a substance that is capable of identifying or detecting a biomarker in a biological sample (e.g., identifies or detects the mRNA of a biomarker, the DNA of a biomarker, the protein of a biomarker). In certain embodiments, the agent is a labeled antibody which specifically binds to a biomarker polypeptide.

In addition, a biomarker can be detected using Mass Spectrometry such as MALDI/TOF (time-of-flight), SELDI/TOF, liquid chromatography-mass spectrometry (LC-MS), gas chromatography-mass spectrometry (GC-MS), high performance liquid chromatography-mass spectrometry (HPLC-MS), capillary electrophoresis-mass spectrometry, nuclear magnetic resonance spectrometry, or tandem mass spectrometry (e.g., MS/MS, MS/MS/MS, ESI-MS/MS, etc.). See for example, U.S. Patent Application Nos: 20030199001, 20030134304, 20030077616, which are herein incorporated by reference.

Mass spectrometry methods are well known in the art and have been used to quantify and/or identify biomolecules, such as proteins (see, e.g., Li et al. (2000) Tibtech 18:151-160; Rowley et al. (2000) Methods 20: 383-397; and Kuster and Mann (1998) Curr. Opin. Structural Biol. 8: 393-400). Further, mass spectrometric techniques have been developed that permit at least partial de novo sequencing of isolated proteins. Chait et al., Science 262:89-92 (1993); Keough et al., Proc. Natl. Acad. Sci. USA. 96:7131-6 (1999); reviewed in Bergman, EXS 88:133-44 (2000).

In certain embodiments, a gas phase ion spectrophotometer is used. In other embodiments, laser-desorption/ionization mass spectrometry is used to analyze the sample. Modem laser desorption/ionization mass spectrometry (“LDI-MS”) can be practiced in two main variations: matrix assisted laser desorption/ionization (“MALDI”) mass spectrometry and surface-enhanced laser desorption/ionization (“SELDI”). In MALDI, the analyte is mixed with a solution containing a matrix, and a drop of the liquid is placed on the surface of a substrate. The matrix solution then co-crystallizes with the biological molecules. The substrate is inserted into the mass spectrometer. Laser energy is directed to the substrate surface where it desorbs and ionizes the biological molecules without significantly fragmenting them. However, MALDI has limitations as an analytical tool. It does not provide means for fractionating the sample, and the matrix material can interfere with detection, especially for low molecular weight analytes. See, e.g., U.S. Pat. No. 5,118,937 (Hillenkamp et al.), and U.S. Pat. No. 5,045,694 (Beavis & Chait).

For additional information regarding mass spectrometers, see, e.g., Principles of Instrumental Analysis, 3rd edition. Skoog, Saunders College Publishing, Philadelphia, 1985; and Kirk-Othmer Encyclopedia of Chemical Technology, 4th ed. Vol. 15 (John Wiley & Sons, New York 1995), pp. 1071-1094.

Detection of the presence of a marker or other substances will typically involve detection of signal intensity. This, in turn, can reflect the quantity and character of a polypeptide bound to the substrate. For example, in certain embodiments, the signal strength of peak values from spectra of a first sample and a second sample can be compared (e.g., visually, by computer analysis etc.), to determine the relative amounts of a particular biomarker. Software programs such as the Biomarker Wizard program (Ciphergen Biosystems, Inc., Fremont, Calif.) can be used to aid in analyzing mass spectra. The mass spectrometers and their techniques are well known to those of skill in the art.

Any person skilled in the art understands, any of the components of a mass spectrometer (e.g., desorption source, mass analyzer, detect, etc.) and varied sample preparations can be combined with other suitable components or preparations described herein, or to those known in the art. For example, in certain embodiments a control sample can contain heavy atoms (e.g., ¹³C) thereby permitting the test sample to be mixed with the known control sample in the same mass spectrometry run.

In certain embodiments, a laser desorption time-of-flight (TOF) mass spectrometer is used. In laser desorption mass spectrometry, a substrate with a bound marker is introduced into an inlet system. The marker is desorbed and ionized into the gas phase by laser from the ionization source. The ions generated are collected by an ion optic assembly, and then in a time-of-flight mass analyzer, ions are accelerated through a short high voltage field and let drift into a high vacuum chamber. At the far end of the high vacuum chamber, the accelerated ions strike a sensitive detector surface at a different time. Since the time-of-flight is a function of the mass of the ions, the elapsed time between ion formation and ion detector impact can be used to identify the presence or absence of molecules of specific mass to charge ratio.

In certain embodiments, the relative amounts of one or more biomarkers present in a first or second sample is determined, in part, by executing an algorithm with a programmable digital computer. The algorithm identifies at least one peak value in the first mass spectrum and the second mass spectrum. The algorithm then compares the signal strength of the peak value of the first mass spectrum to the signal strength of the peak value of the second mass spectrum of the mass spectrum. The relative signal strengths are an indication of the amount of the biomarker that is present in the first and second samples. A standard containing a known amount of a biomarker can be analyzed as the second sample to better quantify the amount of the biomarker present in the first sample. In certain embodiments, the identity of the biomarkers in the first and second sample can also be determined.

### D. Kits

In certain non-limiting embodiments, the present disclosure provides for kits for determining identifying the severity of a patient's AA as well as for identifying and tracking patient sub-populations that will respond to JAK inhibitor treatments. Such kits will, in certain embodiments, include a means for detecting one or more biomarkers selected from the biomarkers set forth herein, or a combination thereof. The disclosure further provides for kits for determining the efficacy of a therapy for treating AA in a subject.

In certain embodiments a kit for treating Alopecia Areata (AA) in a subject comprises one or more detection reagents useful for detecting a biomarker indicative of a disease severity of the subject, and one or more treatment reagents useful for treating AA. The presently disclosed subject matter may further provide for a kit for treating Alopecia Areata (AA) in a subject comprising one or more detection reagents useful for detecting a biomarker indicative of a propensity of the subject to respond to one or more treatment reagent useful for treating AA, and one or more treatment reagents useful for treating AA. In certain embodiments, the kit further comprises one or more probe sets, arrays/microarrays, biomarker-specific antibodies and/or beads. In certain embodiments, the kit further comprises an instruction. In certain embodiments, the treatment reagent may be selected from a JAK inhibitor.

Types of kits include, but are not limited to, packaged probe and primer sets (e.g., TaqMan probe/primer sets), arrays/microarrays, biomarker-specific antibodies and beads, which further contain one or more probes, primers or other detection reagents for detecting one or more biomarkers of the present disclosure.

In a certain, non-limiting embodiment, a kit can include a pair of oligonucleotide primers suitable for polymerase chain reaction (PCR) or nucleic acid sequencing, for detecting one or more biomarker(s) to be identified. A pair of primers can include nucleotide sequences complementary to a biomarker set forth herein, and can be of sufficient length to selectively hybridize with said biomarker. Alternatively, the complementary nucleotides can selectively hybridize to a specific region in close enough proximity 5′ and/or 3′ to the biomarker position to perform PCR and/or sequencing. Multiple biomarker-specific primers can be included in the kit to simultaneously assay large number of biomarkers. The kit can also include one or more polymerases, reverse transcriptase and nucleotide bases, wherein the nucleotide bases can be further detectably labeled.

In certain embodiments, a primer can be at least about 10 nucleotides or at least about 15 nucleotides or at least about 20 nucleotides in length and/or up to about 200 nucleotides or up to about 150 nucleotides or up to about 100 nucleotides or up to about 75 nucleotides or up to about 50 nucleotides in length.

In certain embodiments, the oligonucleotide primers can be immobilized on a solid surface or support, for example, on a nucleic acid microarray, wherein the position of each oligonucleotide primer bound to the solid surface or support is known and identifiable.

In a certain, non-limiting embodiment, a kit can include at least one nucleic acid probe, suitable for in situ hybridization or fluorescent in situ hybridization, for detecting the biomarker(s) to be identified. Such kits will generally include one or more oligonucleotide probes that have specificity for various biomarkers.

In certain non-limiting embodiments, a kit can include a primer for detection of a biomarker by primer extension.

In certain non-limiting embodiments, a kit can include at least one antibody for immunodetection of the biomarker(s) to be identified. Antibodies, both polyclonal and monoclonal, specific for a biomarker, can be prepared using conventional immunization techniques, as will be generally known to those of skill in the art. The immunodetection reagents of the kit can include detectable labels that are associated with, or linked to, the given antibody or antigen itself. Such detectable labels include, for example, chemiluminescent or fluorescent molecules (rhodamine, fluorescein, green fluorescent protein, luciferase, Cy3, Cy5 or ROX), radiolabels (³H, ³⁵S, ³²P, ¹⁴C, ¹³¹I) or enzymes (alkaline phosphatase, horseradish peroxidase).

In a certain non-limiting embodiment, the biomarker-specific antibody can be provided bound to a solid support, such as a column matrix, an array, or well of a microtiter plate. Alternatively, the support can be provided as a separate element of the kit.

In certain non-limiting embodiments, a kit can include one or more primers, probes, microarrays, or antibodies suitable for detecting one or more biomarkers set forth herein or combinations thereof.

In certain non-limiting embodiments, a kit can include one or more primers, probes, microarrays, or antibodies suitable for detecting one, two, three, four, five, six, seven, eight, nine, ten, eleven, twelve, thirteen, fourteen or more of the biomarkers set forth herein.

In certain non-limiting embodiments, where the measurement means in the kit employs an array, the set of biomarkers set forth above can constitute at least 10 percent or at least 20 percent or at least 30 percent or at least 40 percent or at least 50 percent or at least 60 percent or at least 70 percent or at least 80 percent of the species of markers represented on the microarray.

In certain non-limiting embodiments, a biomarker detection kit can include one or more detection reagents and other components (e.g., a buffer, enzymes such as DNA polymerases or ligases, chain extension nucleotides such as deoxynucleotide triphosphates, and in the case of Sanger-type DNA sequencing reactions, chain terminating nucleotides, positive control sequences, negative control sequences, and the like) necessary to carry out an assay or reaction to detect a biomarker. A kit can also include additional components or reagents necessary for the detection of a biomarker, such as secondary antibodies for use in western blotting immunohistochemistry. A kit can further include one or more other biomarkers or reagents for evaluating other prognostic factors, e.g., tumor stage.

A kit can further contain means for comparing the biomarker with a standard, and can include instructions for using the kit to detect the biomarker of interest. For example, the instructions can describe that the presence of a biomarker, set forth herein, is indicative of the severity of a patient's AA, or for identifying and tracking patient sub-populations that will respond to JAK inhibitor treatments.

In certain embodiments, the kit may further include a treatment reagent. In certain embodiments, the treatment reagent may be a JAK inhibitor of embodiments herein.

### E. Reports, Programmed Computers and Systems

The results of a test (e.g., the severity of an individual's AA), or an individual's predicted drug responsiveness (e.g., response to JAK inhibitor therapy), based on assaying one or more biomarkers set forth herein, and/or any other information pertaining to a test, can be referred to herein as a “report.” A tangible report can optionally be generated as part of a testing process (which can be interchangeably referred to herein as “reporting,” or as “providing” a report, “producing” a report or “generating” a report).

Examples of tangible reports can include, but are not limited to, reports in paper (such as computer-generated printouts of test results) or equivalent formats and reports stored on computer readable medium (such as a CD, USB flash drive or other removable storage device, computer hard drive, or computer network server, etc.). Reports, particularly those stored on computer readable medium, can be part of a database, which can optionally be accessible via the internet (such as a database of patient records or genetic information stored on a computer network server, which can be a “secure database” that has security features that limit access to the report, such as to allow only the patient and the patient's medical practitioners to view the report while preventing other unauthorized individuals from viewing the report, for example). In addition to, or as an alternative to, generating a tangible report, reports can also be displayed on a computer screen (or the display of another electronic device or instrument).

A report can include, for example, the severity of an individual's AA, or can just include presence, absence or levels of one or more biomarkers set forth herein (for example, a report on computer readable medium such as a network server can include hyperlink(s) to one or more journal publications or websites that describe the medical/biological implications, such as increased or decreased disease risk, for individuals having certain biomarkers or levels of certain biomarkers). Thus, for example, the report can include disease risk or other medical/biological significance (e.g., drug responsiveness, suggested prophylactic treatment, etc.) as well as optionally also including the biomarker information, or the report can just include biomarker information without including disease risk or other medical/biological significance (such that an individual viewing the report can use the biomarker information to determine the associated disease risk or other medical/biological significance from a source outside of the report itself, such as from a medical practitioner, publication, website, etc., which can optionally be linked to the report such as by a hyperlink).

A report can further be “transmitted” or “communicated” (these terms can be used herein interchangeably), such as to the individual who was tested, a medical practitioner (e.g., a doctor, nurse, clinical laboratory practitioner, genetic counselor, etc.), a healthcare organization, a clinical laboratory and/or any other party or requester intended to view or possess the report. The act of “transmitting” or “communicating” a report can be by any means known in the art, based on the format of the report. Furthermore, “transmitting” or “communicating” a report can include delivering a report (“pushing”) and/or retrieving (“pulling”) a report. For example, reports can be transmitted/communicated by various means, including being physically transferred between parties (such as for reports in paper format) such as by being physically delivered from one party to another, or by being transmitted electronically or in signal form (e.g., via e-mail or over the internet, by facsimile and/or by any wired or wireless communication methods known in the art) such as by being retrieved from a database stored on a computer network server, etc.

In certain exemplary embodiments, the disclosed subject matter provides computers (or other apparatus/devices such as biomedical devices or laboratory instrumentation) programmed to carry out the methods described herein. For example, in certain embodiments, the disclosed subject matter provides a computer programmed to receive (i.e., as input) the identity of the one or more biomarkers disclosed herein, alone or in combination with other biomarkers, and provide (i.e., as output) the disease severity or other result (e.g., drug responsiveness, etc.) based on the level or identity of the biomarker(s). Such output (e.g., communication of disease severity, drug responsiveness, etc.) can be, for example, in the form of a report on computer readable medium, printed in paper form, and/or displayed on a computer screen or other display.

Certain further embodiments of the disclosed subject matter provide a system for determining the severity of an individual's AA, or whether an individual will benefit from JAK inhibitor treatment. Certain exemplary systems include an integrated “loop” in which an individual (or their medical practitioner) requests a determination of such individual's AA severity (or drug response), this determination is carried out by testing a sample from the individual, and then the results of this determination are provided back to the requester. For example, in certain systems, a sample (e.g., skin, blood, etc.) is obtained from an individual for testing (the sample can be obtained by the individual or, for example, by a medical practitioner), the sample is submitted to a laboratory (or other facility) for testing (e.g., determining the biomarker(s) disclosed herein, alone or in combination with one or more other biomarkers), and then the results of the testing are sent to the patient (which optionally can be done by first sending the results to an intermediary, such as a medical practitioner, who then provides or otherwise conveys the results to the individual and/or acts on the results), thereby forming an integrated loop system for determining the severity of an individual's AA (or drug response, etc.). The portions of the system in which the results are transmitted (e.g., between any of a testing facility, a medical practitioner, and/or the individual) can be carried out by way of electronic or signal transmission (e.g., by computer such as via e-mail or the internet, by providing the results on a website or computer network server which can optionally be a secure database, by phone or fax, or by any other wired or wireless transmission methods known in the art).

In certain embodiments, the system is controlled by the individual and/or their medical practitioner in that the individual and/or their medical practitioner requests the test, receives the test results back, and (optionally) acts on the test results to reduce the individual's disease risk, such as by implementing a disease management system.

The various methods described herein, such as correlating the presence or absence or level of a biomarker with an altered (e.g., increased or decreased) severity of AA can be carried out by automated methods such as by using a computer (or other apparatus/devices such as biomedical devices, laboratory instrumentation, or other apparatus/devices having a computer processor) programmed to carry out any of the methods described herein. For example, computer software (which can be interchangeably referred to herein as a computer program) can perform correlating the presence or absence of a biomarker in an individual with an altered (e.g., increased or decreased) severity of AA for the individual. Accordingly, certain embodiments of the disclosed subject matter provide a computer (or other apparatus/device) programmed to carry out any of the methods described herein.

### F. Methods of Treatment

In certain embodiments, a method of treating Alopecia Areata (AA) in a subject comprises identifying the AA disease severity in said subject by detecting a biomarker indicative of said disease severity, and administering a therapeutic intervention to said subject appropriate to the identified disease severity.

In certain embodiments, a method of treating AA in a subject comprising identifying the propensity of a subject having AA to respond to JAK inhibitor treatment by detecting a biomarker indicative of said propensity, and administering a JAK inhibitor to said subject if the identified biomarker indicates a propensity that the subject will respond to said inhibitor.

In certain embodiments, a method of treating alopecia areata in a subject in need thereof comprises administering to the subject a JAK inhibitor; detecting a biomarker indicative of responsiveness to JAK inhibitor treatment; and tailoring administration of the JAK inhibitor based on the responsiveness by either (1) continuing administration of the JAK inhibitor, (2) altering administration of the JAK inhibitor, or (3) discontinuing administration of the JAK inhibitor.

In certain embodiments, the biomarker may be a gene expression signature. In certain embodiments, the gene expression signature comprises gene expression information of one or more of the following groups of genes: KRT-associated genes; CTL-associated genes; and IFN-associated genes. In certain embodiments, the KRT-associated genes comprise DSG4, HOXC31, KRT31, KRT32, KRT33B, KRT82, PKP1 and PKP2. In certain embodiments, the CTL-associated genes comprise CD8A, GZMB, ICOS and PRF1. In certain embodiments, the IFN-associated genes comprise CXCL9, CXCL10, CXCL11, STAT1 and MX1.

In certain embodiments, if one or more CTL-associated genes or one or more IFN-associated genes are downregulated to a set of predetermined gene expression levels, and/or if one or more KRT-associated genes are upregulated to a set of predetermined gene expression levels, the treatment is considered effective and may be continued. In certain embodiments, if a majority of CTL-associated genes and/or a majority of IFN-associated genes are not downregulated to a set of predetermined gene expression levels, and/or if a majority of KRT-associated genes are not upregulated to a set of predetermined gene expression levels, the treatment is considered ineffective and may be discontinued or altered, for example, by administering one or more different JAK inhibitors.

In certain embodiments, the gene expression signature is an Alopecia Areata Disease Activity Index (ALADIN). In certain embodiments, tailoring administration of the JAK inhibitor comprises (1) continuing administration of the JAK inhibitor if each of the CTL score, the IFN score and the KRT score is decreased compared to the scores before treatment, (2) altering administration of the JAK inhibitor if none of the CTL score, the IFN score and the KRT score is decreased compared to the scores before treatment, or (3) discontinuing administration of the JAK inhibitor if each of the CTL score, the IFN score and the KRT score is increased compared to the scores before treatment.

In certain embodiments, the detecting a biomarker indicative of responsiveness to JAK inhibitor treatment is performed before physiological signs of responsiveness to treatment with the JAK inhibitor are present. In certain embodiments, the detecting is performed two weeks to six weeks after treatment with the JAK inhibitor. In certain embodiments, the detecting is performed one week, two weeks, three weeks, four weeks, five weeks, six weeks, one month, two months, three months, four months, five months, six months after treatment with the JAK inhibitor, a combination thereof, or a range between any two of these values.

In certain embodiments, the altering administration of the JAK inhibitor comprises altering the interval of administration, the dosage, the formulation, or a combination thereof. In certain embodiments, the particular JAK inhibitor being administered may be discontinued and a different JAK inhibitor (either in a different class of JAK inhibitors or a different JAK inhibitor in the same class) may be administered.

In certain embodiments, the method further comprises establishing a baseline level of the biomarker indicative of responsiveness to JAK inhibitor treatment before administration of the JAK inhibitor. In certain embodiments, the method further comprises comparing the baseline level with the level after administration to determine the responsiveness to JAK inhibitor treatment before tailoring administration of the JAK inhibitor.

In certain embodiments, said detection of the presently disclosed biomarker is performed on a sample obtained from the subject and the sample is selected from the group consisting of skin, blood, serum, plasma, urine, saliva, sputum, mucus, semen, amniotic fluid, mouth wash and bronchial lavage fluid. In certain embodiments, the subject is human. In certain embodiments, the sample is a skin sample. In certain embodiments, the sample is a serum sample.

In certain embodiments, the detection of the presently disclosed biomarker is performed via a nucleic acid hybridization assay. In certain embodiments, the detection is performed via a microarray analysis. In certain embodiments, the detection is performed via polymerase chain reaction (PCR) or nucleic acid sequencing. In certain embodiments, the biomarker is a protein. In certain embodiments, the presence of the protein is detected using a reagent which specifically binds with the protein. In certain embodiments, the reagent is a monoclonal antibody or antigen-binding fragment thereof, or a polyclonal antibody or antigen-binding fragment thereof. In certain embodiments, the detection is performed via an enzyme-linked immunosorbent assay (ELISA), an immunofluorescence assay or a Western Blot assay.

In certain embodiments, the JAK inhibitor is a compound that interacts with a Jak1/Jak2/Jak3/Tyk2/STAT1/STAT2/STAT3/STAT4/STAT5a/STAT5b/STAT6/OSM/gp 130/LIFR/OSM-Rβ gene or a Jak1/Jak2/Jak3/Tyk2/STAT1/STAT2/STAT3/STAT4/STAT5a/STAT5b/STAT6/OSM/gp130/LIFR/OSM-Rβ protein. In certain embodiments, the JAK inhibitor may be selected from:

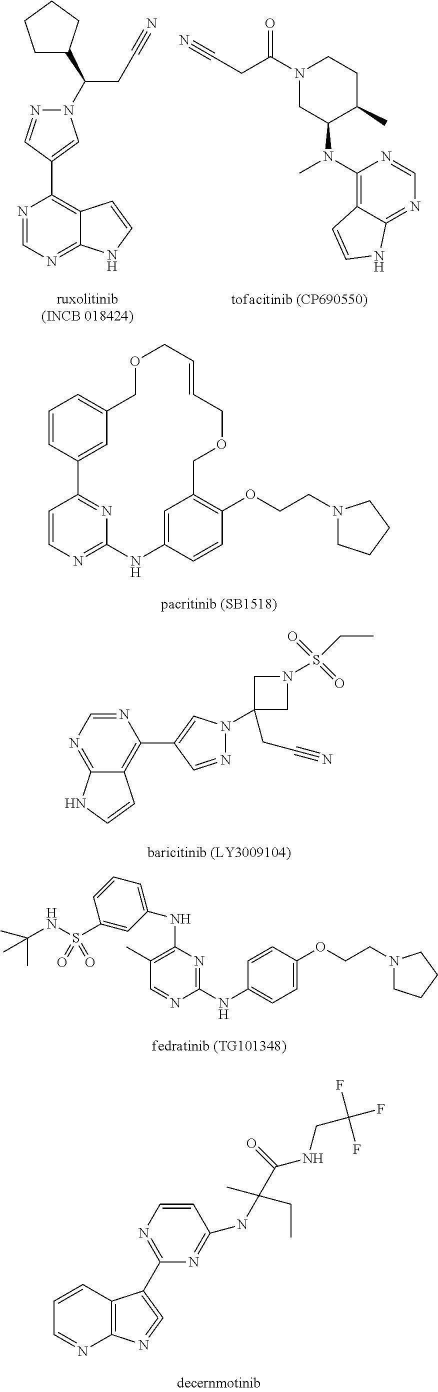

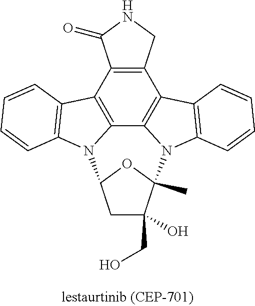 [SMILES: C[C@]12O[C@H](C[C@]1(O)CO)n1c3ccccc3c3c4c(c5c6ccccc6n2c5c31)CNC4=O]

BMS-911543 (CAS Number: 1271022-90-2), fludarabine, epigallocatechin-3-gallate (EGCG), peficitinib, ABT 494 (CAS Number: 1310726-60-3), AT 9283 (CAS Number: 896466-04-9), filgotinib, gandotinib, INCB 39110 (CAS Number: 1334298-90-6), PF 04965842 (CAS Number: 1622902-68-4), R348 (R-932348, CAS Number: 916742-11-5; 1620142-65-5), AZD 1480 (CAS Number: 935666-88-9), cerdulatinib, INCB 052793 (Incyte, clinical trial ID: NCT02265510), NS 018 (CAS Number: 1239358-86-1 (free base); 1239358-85-0 (HCl)), AC 410 (CAS Number: 1361415-84-0 (free base); 1361415-86-2 (HCl).), CT 1578 (SB 1578, CAS Number: 937273-04-6), JTE 052 (Japan Tobacco Inc.), PF 6263276 (Pfizer), R 548 (Rigel), TG 02 (SB 1317, CAS Number: 937270-47-8), lumbricus rebellus extract, ARN 4079 (Arrien Pharmaceuticals, LLC.), AR 13154 (Aerie Pharmaceuticals Inc.), UR 67767 (Palau Pharma S.A.), CS510 (Shenzhen Chipscreen Biosciences Ltd.), VR588 (Vectura Group plc), DNX 04042 (Dynamix Pharmaceuticals/Clevexel), hyperforin, a derivative thereof, a deuterated variation thereof, a salt thereof, or a combination thereof. In certain embodiments, the detection reagent may be selected from a fluorescent reagent, a luminescent reagent, a dye, a radioisotope, a derivative thereof or a combination thereof.

### 6. EXAMPLES

The following examples are put forth so as to provide those of ordinary skill in the art with a disclosure and description of how to make and use the subject matter of the instant application. The following examples are not intended to limit the scope of what the inventors regard as the presently disclosed subject matter. It is understood that various other embodiments may be practiced, given the general description provided above.

### 6.1 Example 1

A. Introduction

Alopecia areata (AA) is an autoimmune skin disease in which the hair follicle is the target of immune attack. Patients characteristically present with round or ovoid patches of hair loss usually on the scalp that can spontaneously resolve, persist, or progress to involve the scalp or the entire body. The three major phenotypic variants of the disease are patchy-type AA (AAP), which is often localized to small ovoid areas on the scalp or in the beard area, alopecia totalis (AT), which involves the entire scalp, and alopecia universalis (AU), which involves the entire body surface area. There are currently no FDA approved drugs for AA, and treatment is often empiric but typically involves observation, intralesional steroids, topical immunotherapy or broad immunosuppressive treatments of unproven efficacy. The more severe forms of the disease, AU and AT, are often recalcitrant to treatment. Furthermore, a prevailing assumption among dermatologists and treating physicians is that long-standing AU and AT becomes irrecoverable, or transforms the scalp to a “burned out” state, supported by an inverse correlation between disease duration and responsiveness to treatment. Despite its high prevalence and the need for effective treatments, the identities of the molecular and cellular effectors of the disease had not been well studied.

Recent strides in the field have transformed the understanding of disease pathogenesis, drug targets, and potential therapeutic solutions. Of particular note are single nucleotide polymorphisms associated with AA that suggest that polymorphisms in ULBP3 and ULBP6 confer an increased risk for developing the disease. The ULBP family of genes encode proteins that serve as ligands for NKG2D and, when expressed, mark a cell for immune targeting by natural killer cells or NKG2D-expressing CD8 T cells. These data led to the recognition of NKG2D-bearing CD8 T cells in the peribulbar infiltrate in skin sections of lesional scalp biopsy specimens of patients with AA as well as in affected skin and skin-draining lymph nodes from the C3H/HeJ mouse model of spontaneous AA. Adoptive transfer of this population of cells from C3H/HeJ mice with alopecia into unaffected C3H/HeJ mice led to the induction of alopecia, substantiating a pivotal role for these effector cells in the mouse AA model.

The inventors previously identified a prominent interferon (IFN) and common gamma chain cytokine (γc) signatures, both of which were hypothesized to contribute to AA pathogenesis. Based on these findings, a therapeutic strategy based on inhibition of critical members of a family of signaling molecules, Janus kinases (JAKs), was found to be effective at treating AA in a mouse model of disease and a small series of human patients. Gene expression profiling played a critical role in the selection of small molecule JAK inhibitors for AA, and expanded efforts in this regard that include the different AA phenotypes have the potential to provide additional insights into novel therapeutic solutions as well as pathogenic mechanisms.

In this study, over 120 samples collected from a total of 96 patients with a range of AA phenotypes and normal control patients were profiled. Patient samples were collected from the National Alopecia Areata Registry sites across the United States after phenotypic classification by dermatologists who specialize in hair disorders. Skin biopsy samples were then interrogated using microarray-based gene expression analysis to identify the AA-specific gene expression signature. Despite a prevailing notion that AT/AU is a “burned-out” form of disease, or is irrecoverable, a striking amount of immune activity in AT/AU samples by gene expression analysis was found, signifying the possibility that treatments that disrupt this immune activity may be useful for therapeutic purposes. Furthermore, based on the data, an Alopecia Areata Disease Severity Index (ALADIN) was created, which was a gene expression metric that effectively distinguishes AT/AU samples, AAP samples, and NC samples from each other and may be used to track disease activity in patients undergoing conventional or experimental treatments.

B. Results

AA Gene Signatures

Gene expression profiling was performed on 122 samples from 96 patients comprised of a discovery dataset of 63 patients and an external validation dataset of 33 patients (for a more complete description refer to Methods section). Microarray-based gene expression analysis was conducted on the discovery dataset, consisting of 20 AAP, 20 AT/AU, and 23 normal control scalp skin biopsy specimens. Differentially expressed genes were identified based on the comparison of AA samples versus normal controls. In order to ensure the robustness of the data from this initial set of samples, external validation was performed using an additional 8 AAP, 12 AT/AU, and 13 normal control scalp skin biopsy specimens as a validation set. From this set of analyses, a disease specific gene expression profile was generated, based on differentially expressed genes selected with an absolute fold change (FC) >1.5 and false discovery rate (FDR)<0.05. The AA-specific disease signature was comprised of 1083 Affymetrix probes that showed increased expression and 919 Affymetrix probes that showed decreased expression in AA.

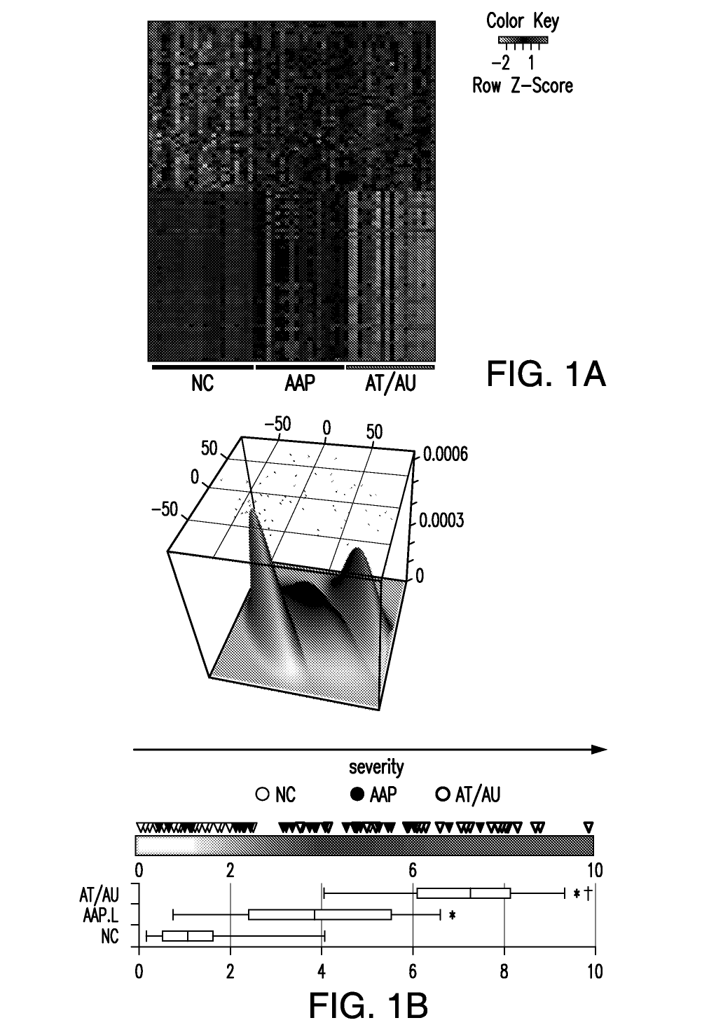

Of note, genes associated with cell mediated cytotoxicity including PRF1 and several granzymes, as well as immune cell trafficking chemokine genes were among the top genes listed as showing increased expression, while hair keratin associated genes and developmental genes such as DSMG4, FGF18, and GPRC5D were among those genes showing decreased expression. Patterns of gene expression distinguished the phenotypic groups from each other, with normal controls and AT/AU samples showing the greatest disparity (FIG. 1A). Plotting the samples in a terrain expression map revealed three clusters corresponding to healthy controls, AAP patients, and AT/AU patients. These patient groups fell along a near-linear path through the terrain map (FIG. 1B).

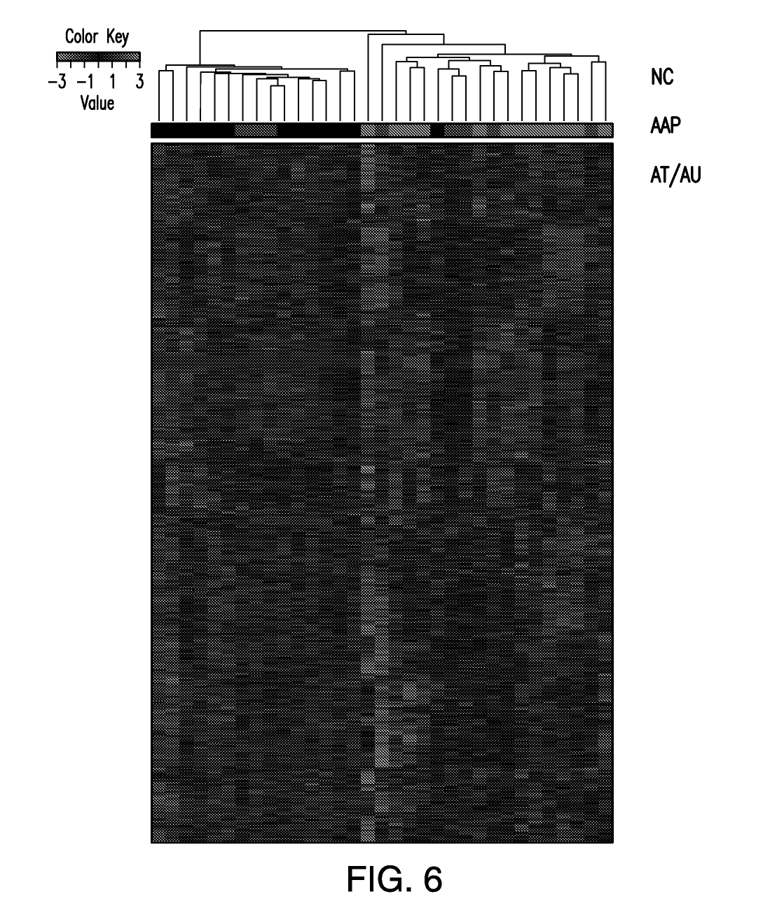

A single score was generated evaluating the relative risk of any given sample being AAP or AT/AU based on its location in this terrain. This score is, by extension, based on a consensus of all differentially expressed genes between AA and healthy controls (see Methods). The resulting score is bounded between 0-10, 10 representing risk of maximal severity (AT/AU), and 0 represent minimal risk (healthy controls). AAP samples fell in a middle range between these two extremes (score range 2-6). Both AAP and AT/AU cohorts had statistically separable average scores compared to healthy controls (FIG. 1B box-and-whiskers plot). The differentially expressed genes from the discovery data set were able to distinguish the AA samples from normal samples by hierarchical clustering in the validation set (FIG. 6). These data suggest the pathology of AA can be expressed at the level of molecular gene expression, and that AAP samples exhibit an AA-specific signature that is intermediate between that for AT/AU and normal controls.

AT/AU Skin Samples are Immunologically Active

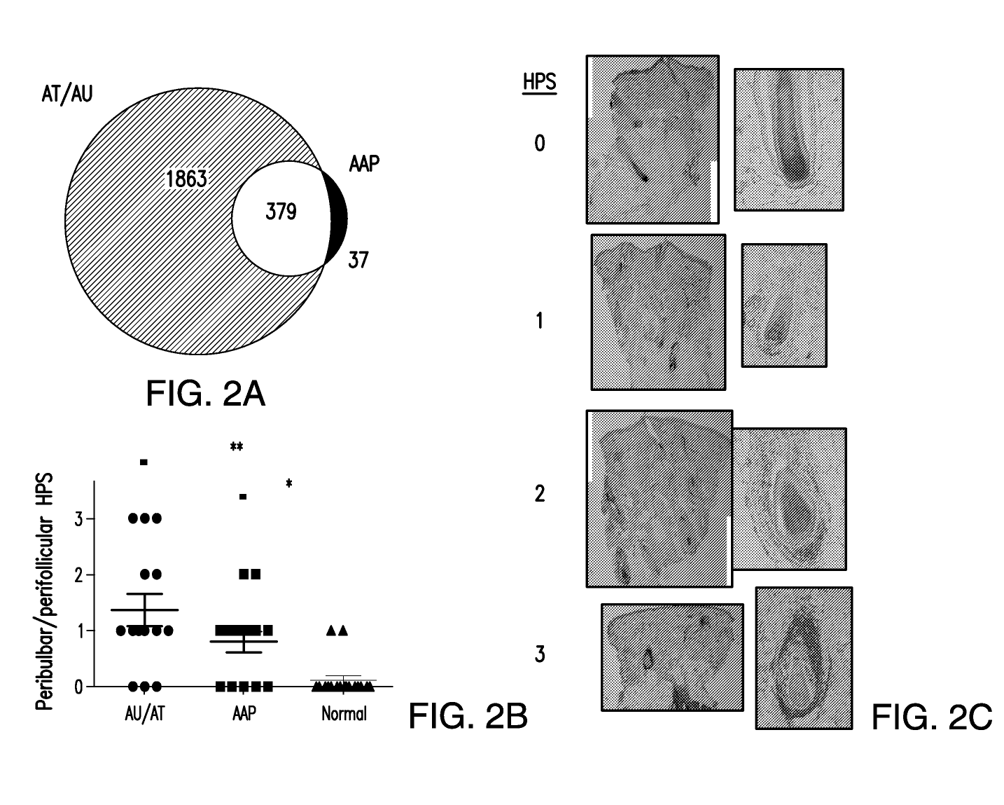

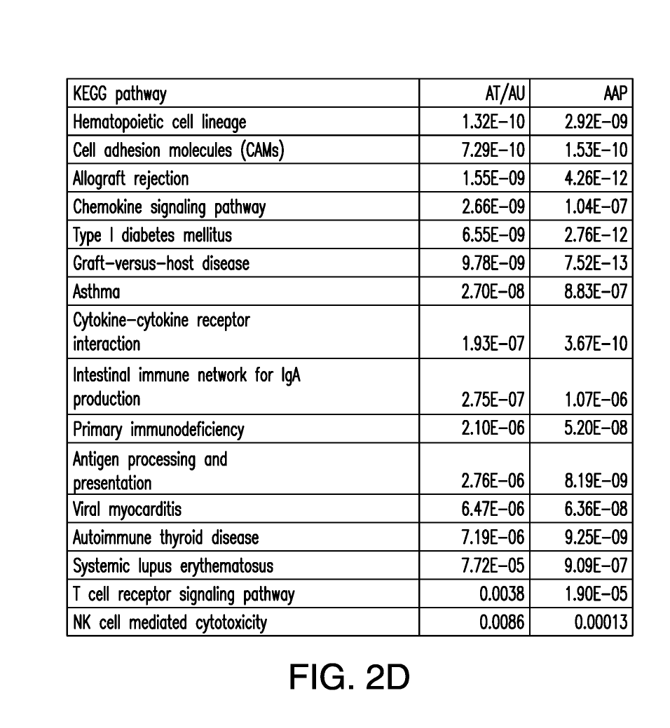

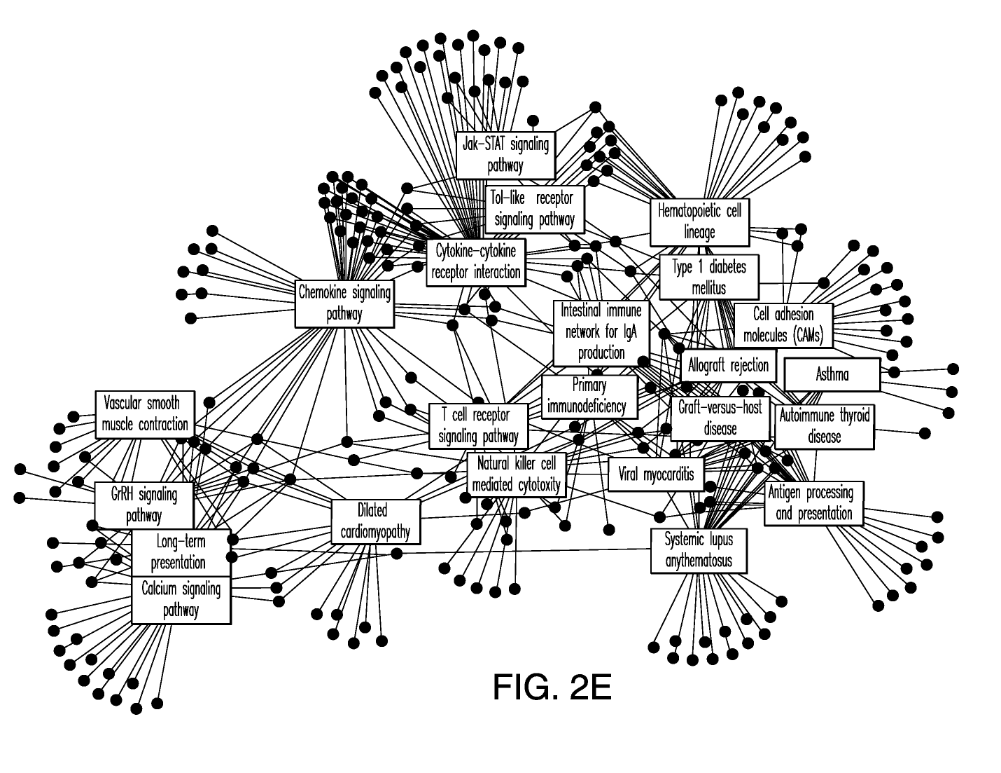

The linear presentation of molecular classification between controls, AAP, and AT/AU in global gene expression analyses, in combination with the presence of immune-related genes in the disease signature, led us to question whether AT/AU samples were immunologically active. Because AT/AU samples seemed to exhibit a more severe AA-specific signature than those of AAP based on both the level of differential expression and the number of differentially expressed genes, the gene expression profiles of AT/AU compared with normal as well as that for AAP compared with healthy controls were separately examined. The AT/AU-specific disease signature, based on FC >1.5 and FDR <0.05, was comprised of 2239 genes with increased expression and 1643 genes with decreased expression. The AAP-specific disease signature, based on similar thresholds, exhibited much lower numbers of differentially expressed genes, with only 376 Affymetrix probes with increased expression and 537 Affymetrix probes with decreased expression. Comparison of the AT/AU- and AAP-specific genes lists showed overlap of AAP-specific genes among the two lists, with few AAP-specific genes not contained within the AT/AU-specific gene list (FIG. 2A). These data along with the prior data indicate that AT/AU is more complex and more severe than the more localized AAP form of the disease, in contrast to the hypothesis that AT/AU is a “burned-out” state of disease.

Noting more robust expression of immune associated genes, the inventors sought to corroborate the more severe gene expression profile seen in AT/AU samples using another method. In order to determine the extent to which infiltration by T cells, the most abundant and most functionally relevant infiltrating immune cell in AA, was observed in AT/AU samples compared with AAP samples, immunohistochemical staining for the pan-T cell marker CD3 was performed on AT/AU, AAP, and NC samples. AT/AU samples exhibited a significant increase in the relative amount of immune infiltration compared with that of NC samples (FIG. 2B), and a trend towards increased infiltration compared with AAP samples was additionally observed using a histopathological scoring system of peribulbar/perifollicular infiltration (FIG. 2C). These data indicate that AT/AU samples exhibit a high and sustained amount of immune activity and inflammation.

Pathway analysis was performed for signatures that were upregulated in either AAP or AT/AU samples. Interestingly, the shared set of pathways that were upregulated in both AAP and AU/AT (FIGS. 2D-2E), including “Graft-versus-host disease,” “Type I diabetes mellitus,” “Allograft rejection,” “Cell adhesion molecules,” and “Antigen processing and presentation,” were made up of antigen presentation genes, supporting the pathogenic theme of loss of immune privilege of the hair follicle microenvironment and immune activation in AA. Interestingly, the “Chemokine signaling pathway” was also found to be significantly upregulated, raising the possibility of targeting these intercellular trafficking molecules for therapeutic purposes, as has been proposed for other autoimmune skin diseases. These results indicate that the majority of the active immune pathways in AA are the same in the milder as well as the more severe forms of the disease.

Paired Lesional and Nonlesional Samples are Similar in Gene Expression Profiles

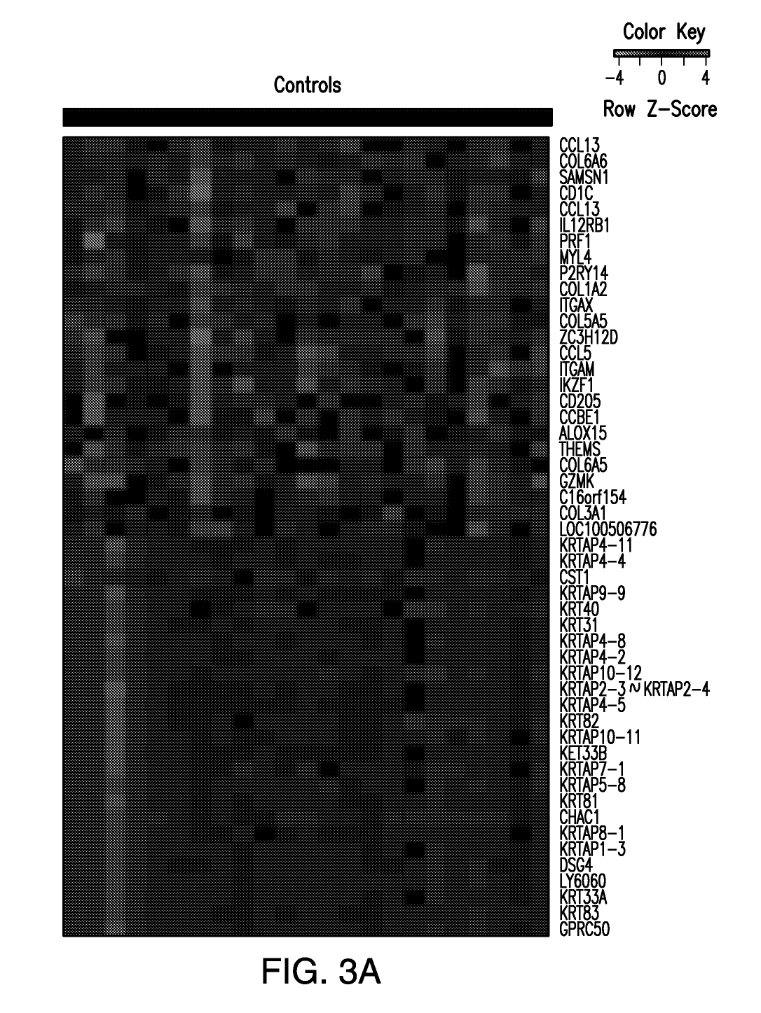

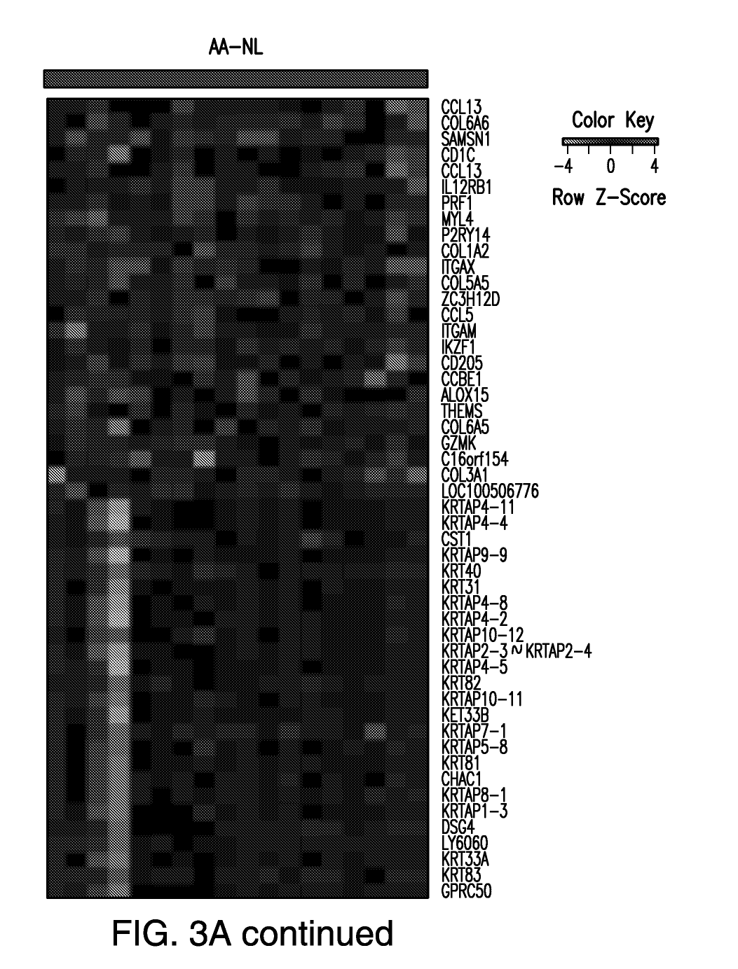

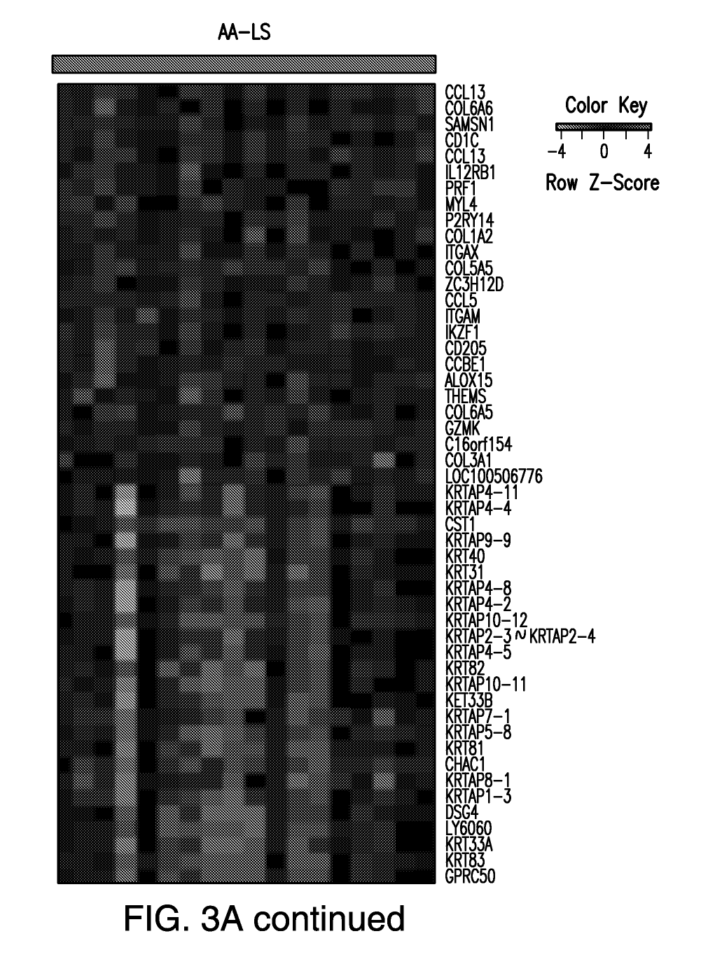

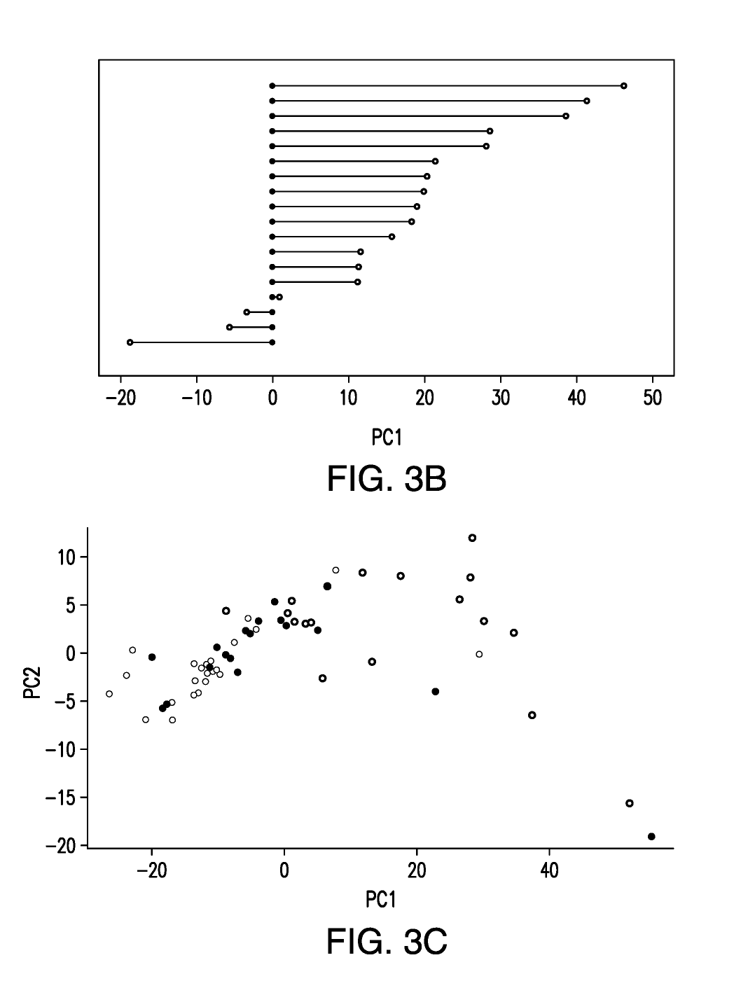

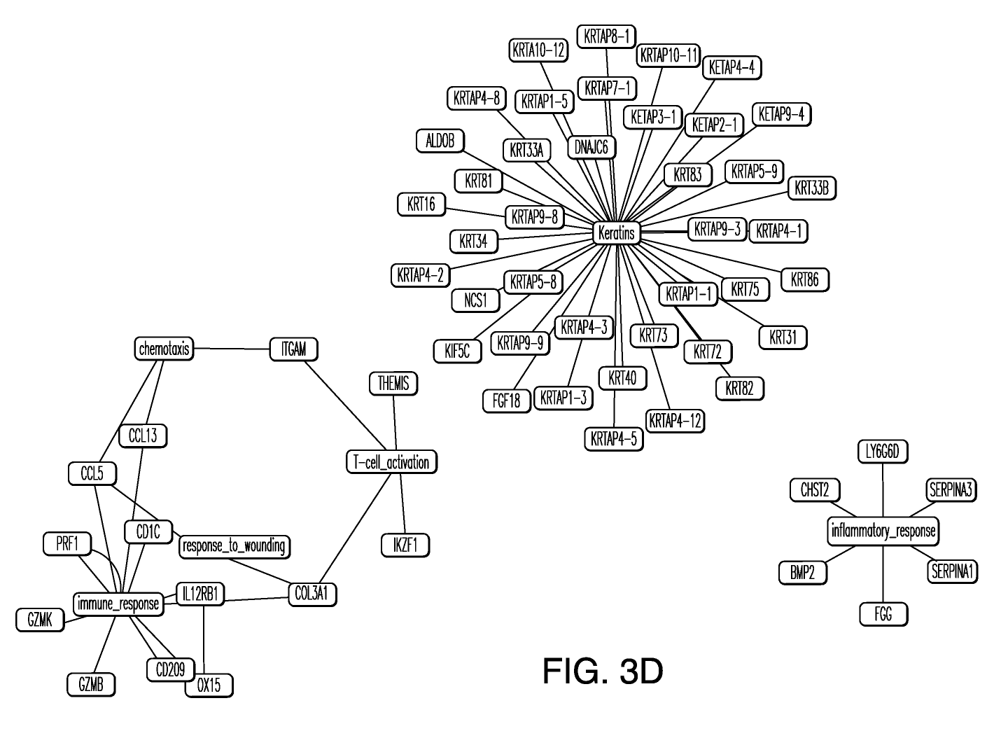

It is unclear whether the AA-specific gene signature is present following onset of symptoms, or rather present as part of a global signature that could be used to differentiate an AA subject from an unaffected subject. In order to test whether nonlesional skin from an AA patient is significantly different from skin from a normal patient, an additional 18 samples of nonlesional skin (AAP-NL) from AA patients were analyzed with corresponding lesional skin (AAP-LS) as part of the training set and 8 additional AAP-NL samples with corresponding AAP-LS were used for the validation set. Differentially expressed genes between AAP-LS skin and AAP-NL were determined, based on FC >1.5 and p-value <0.05, with 27 genes showing increased expression and 143 genes showing decreased expression in AAP-LS. Examination of the level of expression of this set of genes indicated that AAP-NL exhibited a profile that was intermediate between AAP-LS and NC (FIG. 3A).

To more clearly visualize the differences between these three populations detectable by gene expression, a principal component (PC) analysis was performed. This analysis attempts to identify the fewest dimensions in highly complex data that maximally separate the samples based on gene expression. The end result of this analysis is that samples with similar gene expression profiles cluster closely together and samples with dissimilar profiles cluster separately from each other. Based on the first component of a PC analysis, non-lesional samples (AAP-NL) exhibited highly variable dissimilarity from patient-matched lesional samples (AAP-LS, FIG. 3B). Arraying all samples against the first two principal components was consistent with this data, with AAP-LS and NC samples exhibiting disparate clustering, and AAP-NL exhibiting a profile that was intermediate between AAP-LS and NC (FIG. 3C). A plot of the first component of a PC analysis of the 8 AAP-L/AAP-NL pairs from the validation dataset showed the same highly variable dissimilarity across pairs that was observed in the discovery dataset. The genes differentially expressed between non-lesional and lesional samples were analyzed for functional annotations, and found that the most common genes present in non-lesional samples (but absent from lesional samples) were hair-associated keratins and a handful of inflammatory response genes (FIG. 3D). Genes associated with immune response and infiltration, including CCL5/13, PRF1, GZMB/K, ITGAM, and CD209 were missing from the non-lesional samples. These results indicate that while non-lesional sample biopsies from AA patients do not entirely resemble the lesional regions, they also do not resemble healthy control scalp skin. These samples exist in an intermediate state that is different than normal, unaffected samples, but has not yet elicited a full autoimmune response, as seen in lesional samples.

Infiltrate Gene Expression Signatures Correlate with AA Phenotype

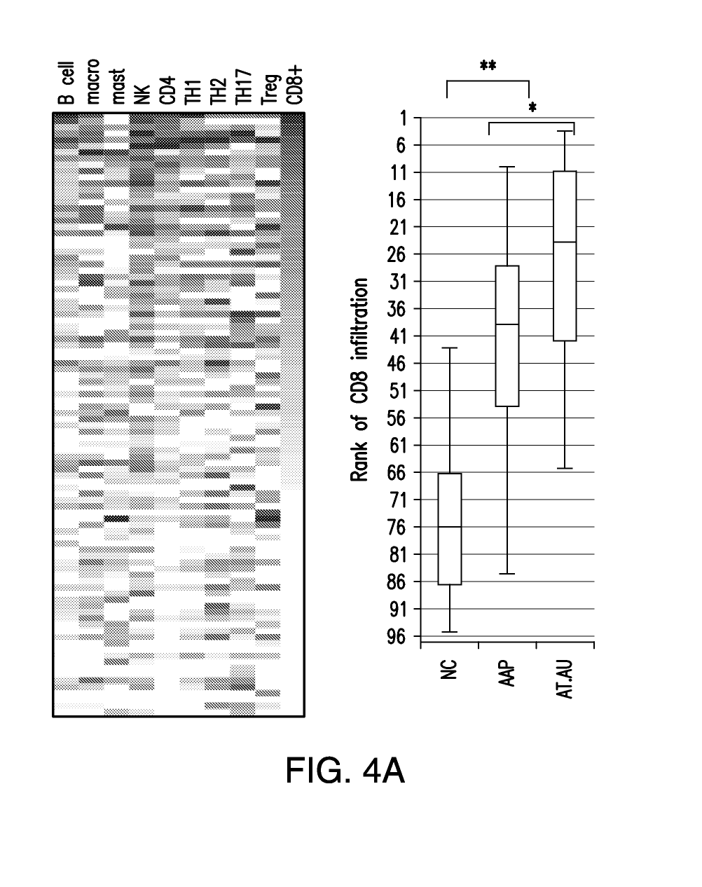

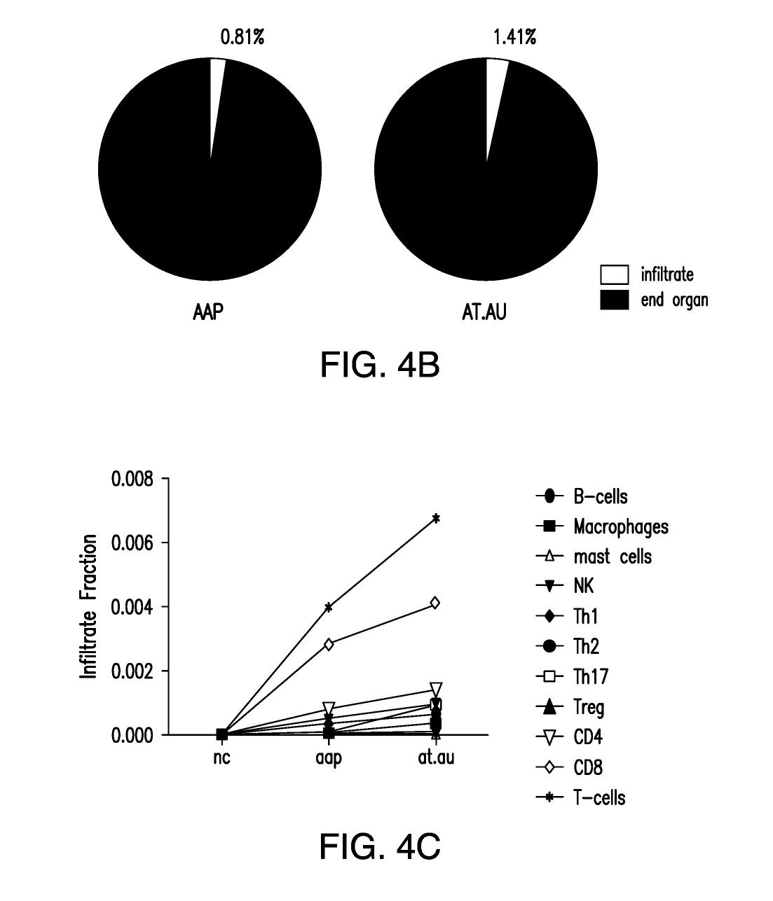

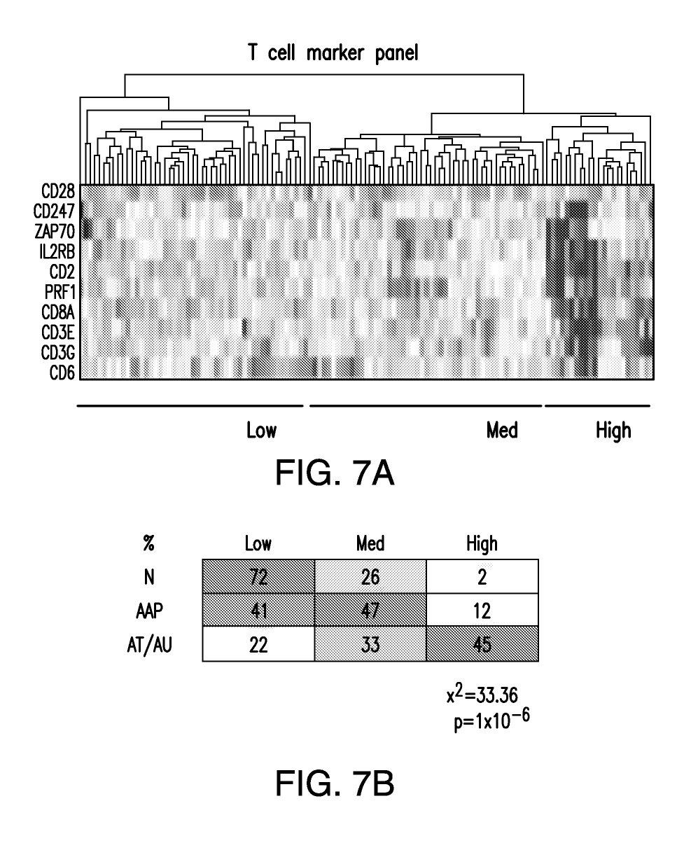

The presence of significant infiltrates in both the AAP and AT/AU patient cohorts and the presence of immune-related marker genes in the gene expression array cohorts led us to question whether or not these infiltrates could be detected directly in microarray analysis. The ability to detect infiltrating populations would prove informative to the pathology and characterization of AA. To identify any infiltrating immune tissues, unique gene expression signatures were adopted defining each of several infiltrating immune cells and used as the Immune Gene Signature (IGS). This work provided comprehensive, mutually exclusive gene markers for several immune types including but not limited to B-cells, T-cells, macrophages, natural killers, and mast cells. Numeric relative measurements of the relative infiltration of each of these tissue types were built as a function of the expression of the corresponding IGS (FIG. 4A). Using this metric, the inventors were able to quantitate the relative infiltration of each immune tissue on a patient-by-patient basis and test them for correlation with AA onset and severity (FIG. 7A-7B). Of the infiltrates tested, only ranking of CD8 T-cells and natural killer cells had power to segregate NC from AAP or AT/AU. Ranking by CD8 activity produced a dose-dependent separation between the three clinical presentations, significantly separating the three populations (hashes represent the medians of each cohort). NK-specific markers did not mirror the power of CD8 T cell-specific markers, indicating that the correlation is not likely the result of NK infiltrates or shared NK/CD8 T cell genes.

Using the IGS metrics, the inventors also estimated the overall infiltrate signal contaminating the AAP samples (FIG. 4B, left pie), and the AT/AU samples (FIG. 4B, right pie). The overall estimated changes in infiltration of each immune tissue type is also presented (FIG. 4B, chart). From the gene expression data, an estimated infiltrate contamination of 0.8-1.4% were observed, correlating with increased clinical severity of AA. Concordantly, CD8⁺ infiltrates consisted of greater than 65% of the total infiltrate load only in samples from AAP or AT/AU patients. The absolute change in each immune tissue infiltrate across the three presentations is also shown (FIG. 4C), indicating that only CD8⁺ infiltrates change significantly across the three populations. These results indicate that, although there is some expression-based evidence for multiple infiltrating tissue types, the most significantly present type associated to AA are non-NK, CD8⁺ cells. In addition, the inventors were able to detect elevated levels of markers associated with macrophages, total CD4⁺ T cells, CD4⁺ T cell subsets, NK cells and B cells, though these represented minor fractions compared to the CD8 T cell fraction. Overall, the contamination within each sample is relatively small—the entire population of tested immune tissues do not exceed ˜3% of the total signal.

Furthermore, the IGS scores were used to estimate the relative Th1 and Th2 fractions detected in patient samples (FIG. 4D). For each patient (AA or unaffected control), the Th load within the sample biopsy was represented as a ratio of Th1:Th2 signal, and observed that AA patient samples exhibit a shift to higher Th1 ratios compared to normal controls. The rank shift of Th1:Th2 associated with AA presentation was statistically significant by the Mann-Whitney U-test (p=1.02×10−4) indicating that, on the whole, skin from AA patients contains elevated levels of Th1 signatures relative to Th2 signatures as compared with unaffected patients, though there are AA patients with both Th1 and Th2 signatures.

ALADIN Scores Parallels Disease Phenotype

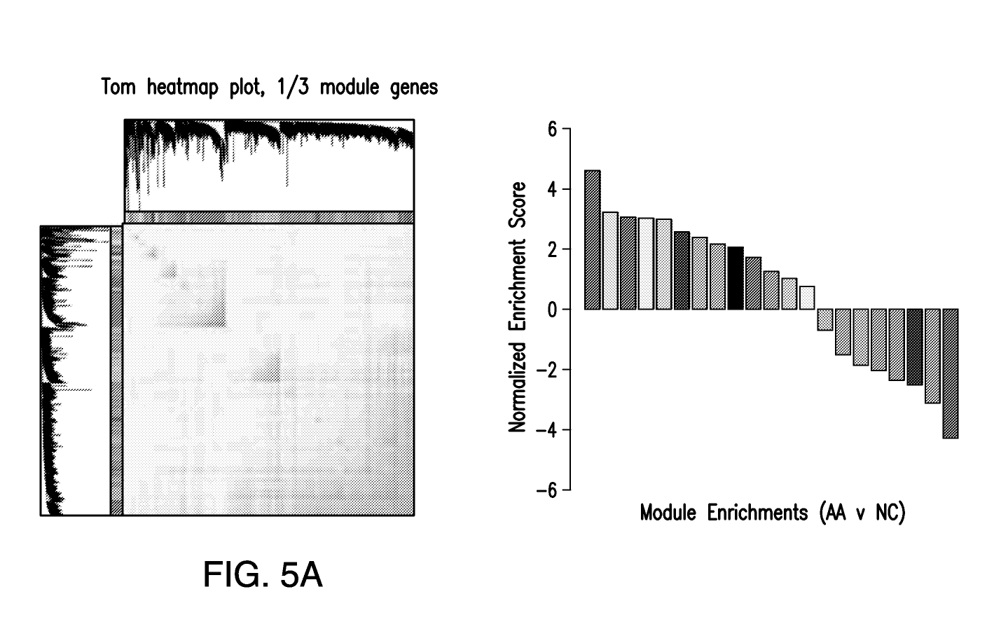

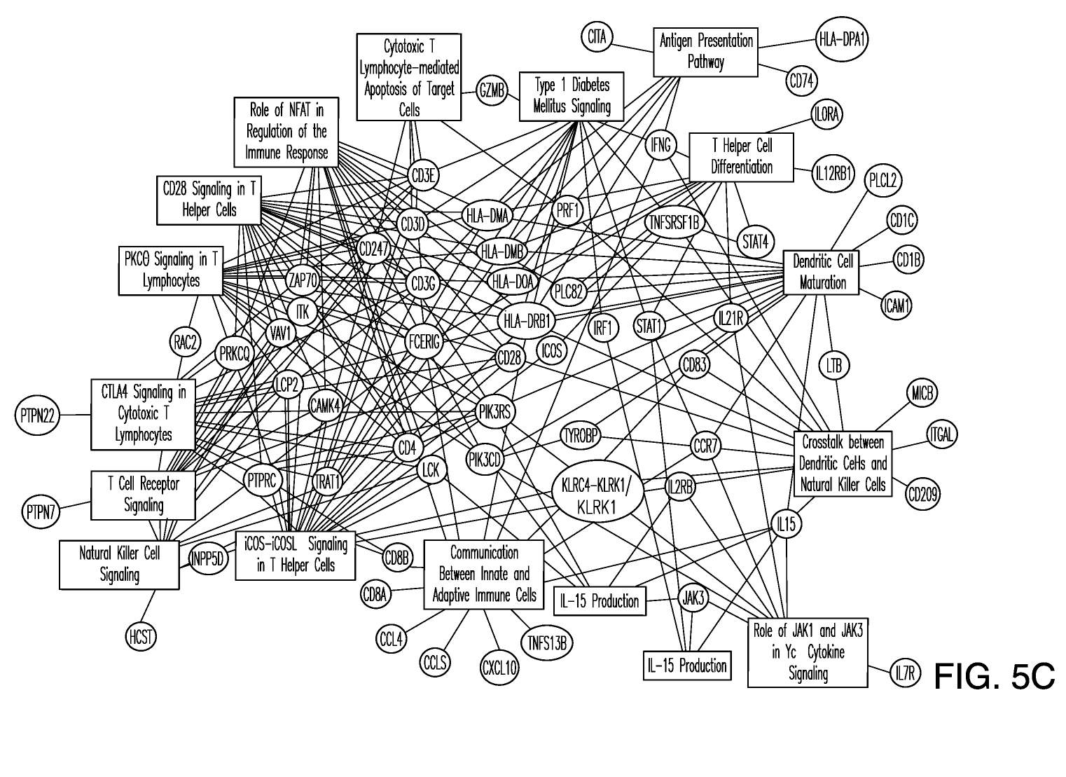

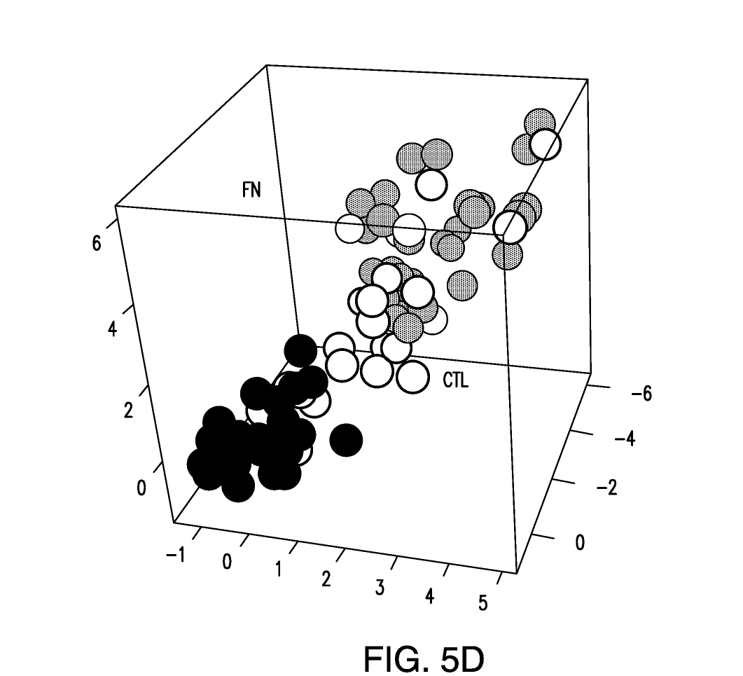

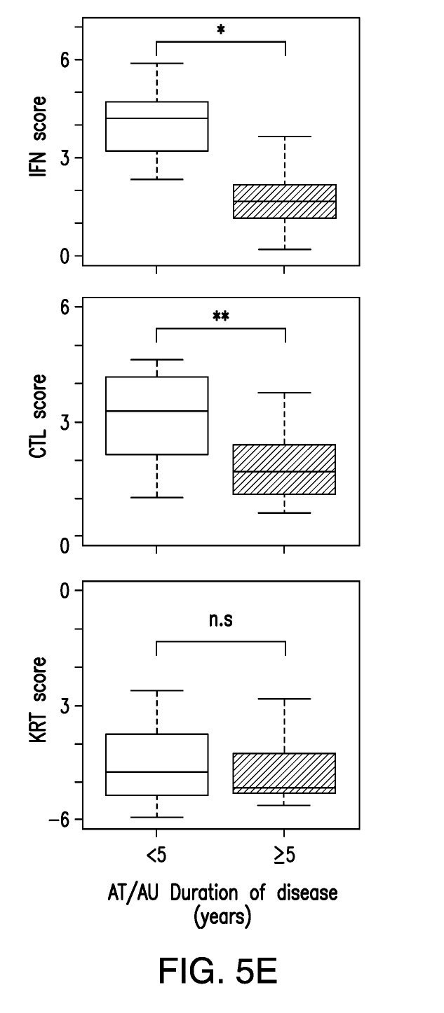

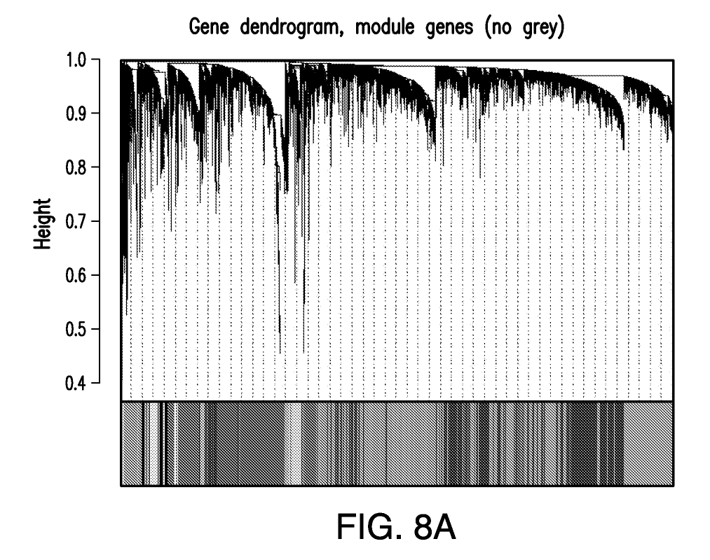

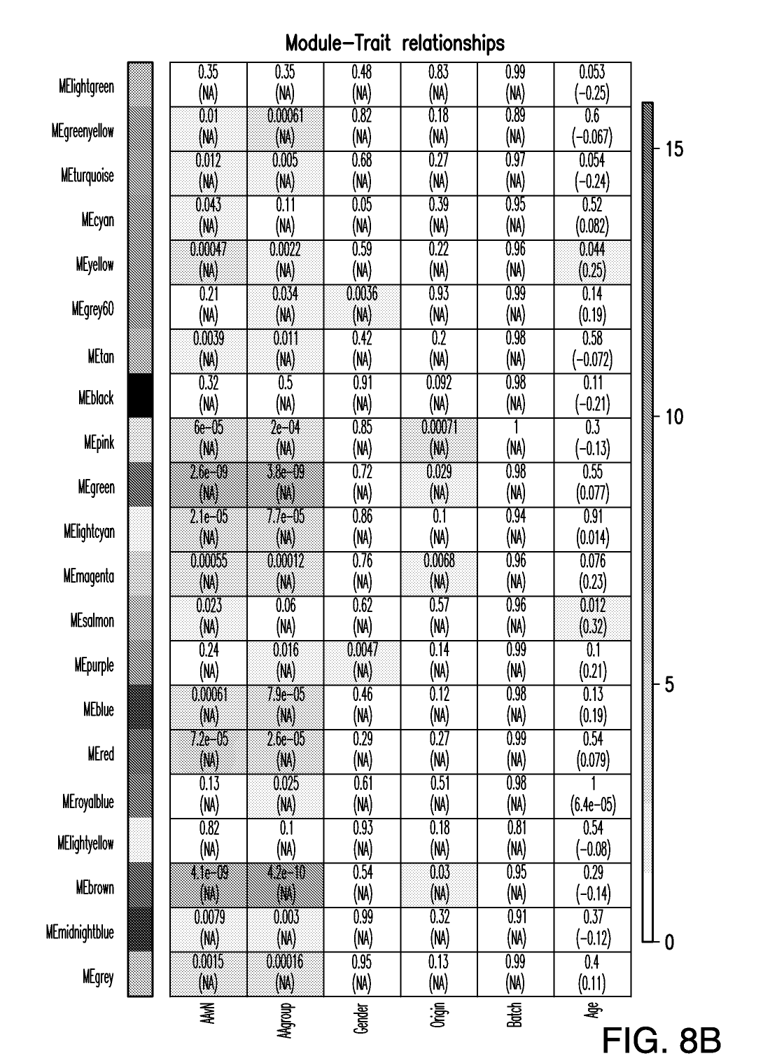

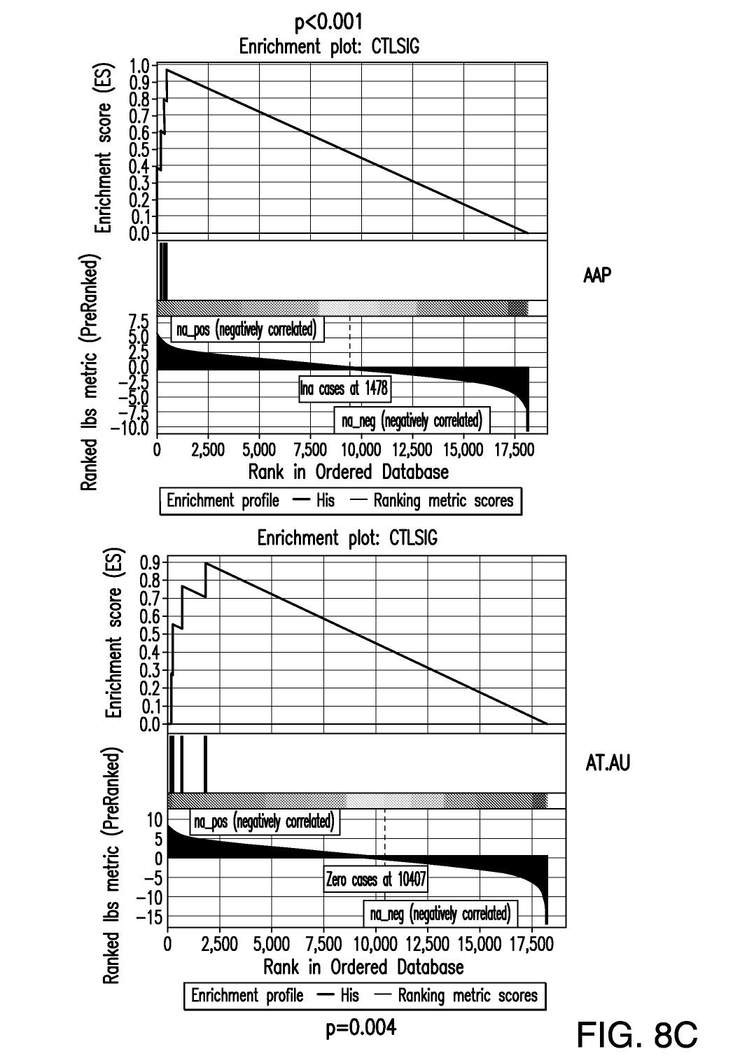

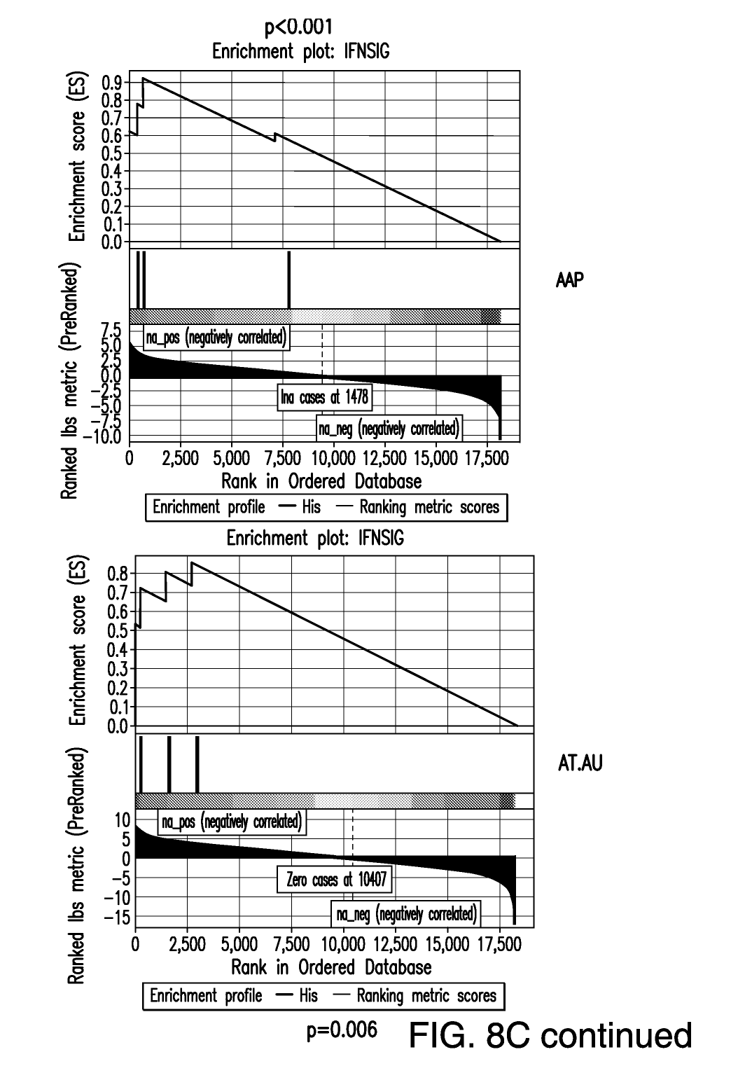

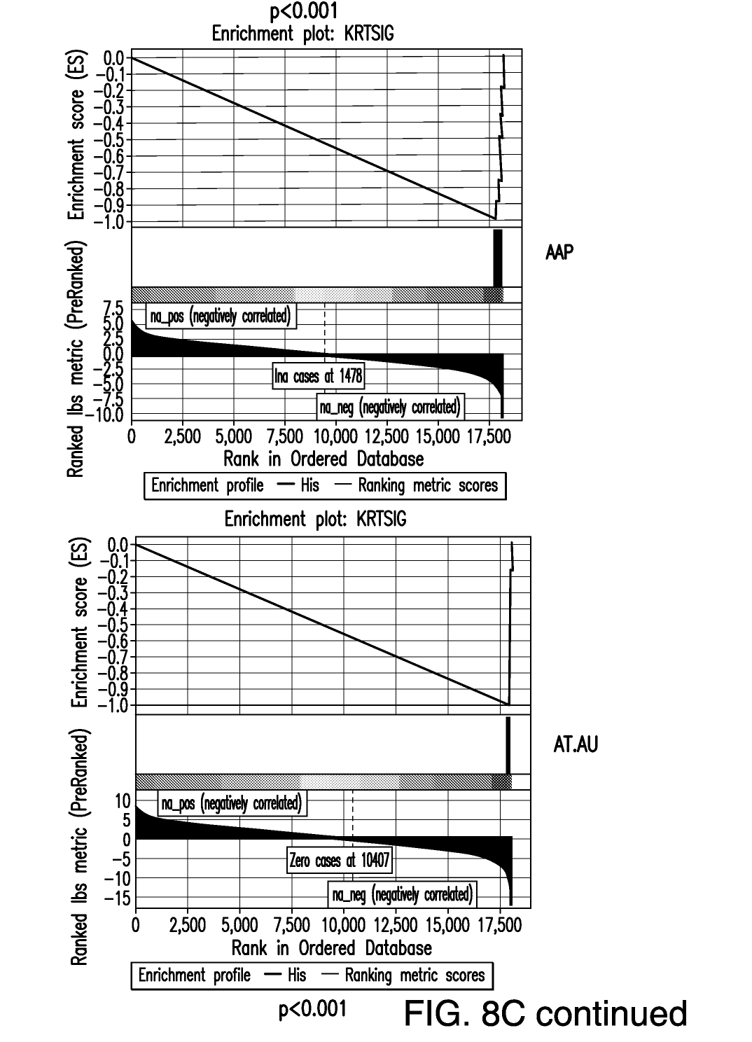

The inventors sought to generate a metric that identified the most prominent features of the AA disease signature that would allow for a quantitative assessment of disease status. Weighted gene co-expression analysis (WGCNA) of the genes differentially expressed between AA and healthy controls revealed 20 clusters of co-expressed genes (FIG. 5A). These gene sets represent co-expressed modules and indicate the possibility of co-regulation, shared biological function, and/or shared pathways. For each of these modules the inventors are able to define color-coded eigengenes, or metagenes, using the first principal component of the gene expression signature derived from the genes within each module. Gene set enrichment analysis (GSEA) of these modules with ranked lists of genes that were differentially expressed between AA and NC cohorts, as well as tests of association between module metagenes and disease phenotype revealed that the green and brown modules are the most significantly associated with disease phenotype and that these modules (FIGS. 5B, 8A and 8B). These contain immune and immune response signatures (green) and structural keratins (brown). Pathway enrichment analysis of the green module revealed several gene pathways associated with autoimmune response (FIG. 5C). This included genes such as CD8, CD4, MICB, CCL4/5, CCR7, and ICOS. Both perforin and granzyme B were detected, as well as genes previously implicated by the GWAS meta-analysis including ICOS, IRF1, and CIITA.

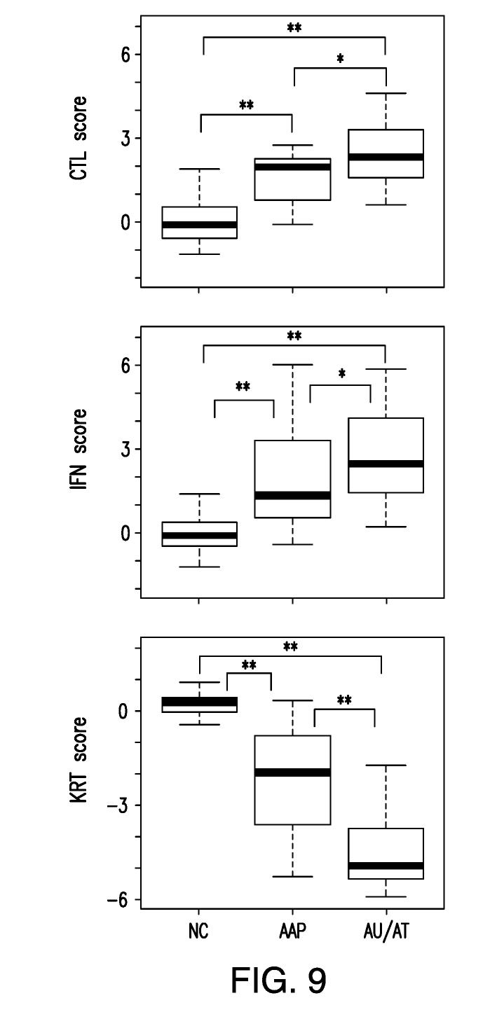

An original scoring system, the Alopecia Areata Disease Activity Index (ALADIN) was developed, which was a three-dimensional quantitative composite gene expression score, for potential use as a biomarker for tracking disease severity and response to treatment. The metric scores patients along a combination of cytotoxic T lymphocyte infiltration (CTL), IFN-associated markers (IFN), and a hair keratin panel (KRT). Interestingly, the CTL signature contains the two genes, CD8A and PRF1, which make up the CD8 T-cell signature above (FIG. 4). Inspection of the components of the green module revealed the presence of genes contained in both the ALADIN CTL and IFN signatures, and the brown signature contained the genes that made up the ALADIN KRT signature. Patient CTL, IFN and KRT scores were determined to test the robustness of ALADIN to a new dataset (FIG. 9). A three-dimensional plot of the ALADIN scores for the combined discovery and validation dataset of 96 AT/AU, AAP, and NC samples showed that AT/AU samples clustered farthest away from NC samples, with AAP samples positioned in an intermediate position between both of these sets (FIG. 5D). A subsequent GSEA showed statistically significant enrichment of the original ALADIN gene sets in AA samples compared with normal controls in both AAP and AT/AU cohorts (FIG. 8C). These data indicate that the ALADIN score may distinguish AA forms that differ in severity and invites the use of this metric in clinical trials.

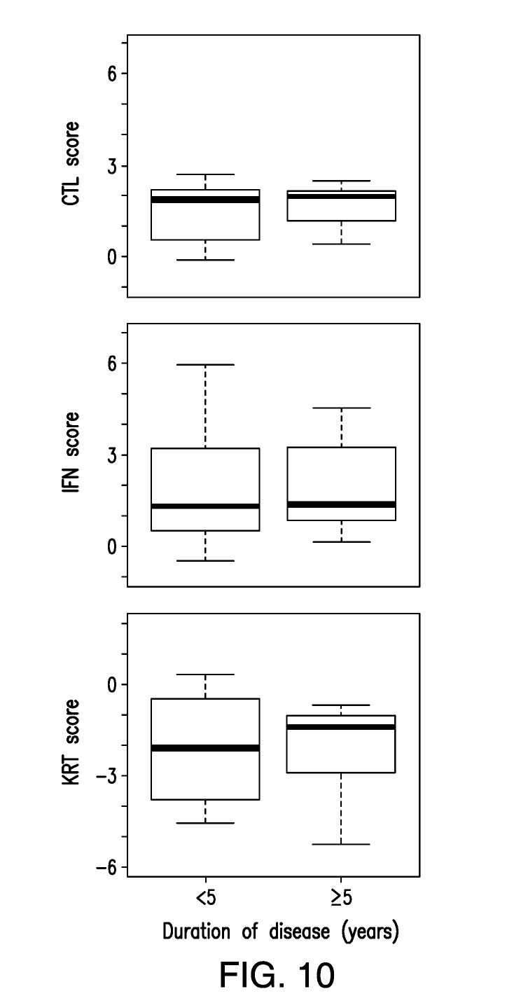

Further, the inventors assessed whether or not the duration of disease influenced the ALADIN score. Skin samples from AT/AU patients with 5 or more years of disease exhibited statistically significant decreases in IFN and CTL scores when compared with samples from AT/AU patients of shorter duration (FIG. 5E). This relationship was not seen between long- and short-duration AAP samples (FIG. 10). These data indicate that the ALADIN score may distinguish AA forms that differ in severity, and, further, that inflammatory and immune infiltrate scores diminish among the more severe forms of AA over time.

C. Discussion

Microarray based whole genome gene expression assays were utilized to make fundamental insights into the biology of AA. The work here includes the use of over 120 scalp skin biopsy specimens from patients with AA and healthy controls. The inventors utilize this method for the first time to identify several critical features of disease pathogenesis.

First, AT/AU exhibits a relatively high level of immune activity compared with normal controls and AAP samples. The notion that patients with AT/AU cannot be effectively treated likely stems from a historical difficulty in treating these patients with previously available topical and oral medications and difficulty in identifying appreciable numbers of rudimentary hairs in skin biopsy specimens of patients with severe disease. However, the data challenge this idea by providing evidence for sustained immunological activity in AT/AU samples that is equal to (if not greater than) that seen in AAP. This immune activity in patients with AT/AU, in combination with anecdotal reports, albeit rare, of spontaneous resolution of AT/AU disease, implies that a sufficiently strong immunosuppressant or treatment targeting a pathway necessary for the maintenance of the immune response may be efficacious for these types of patients. Indeed, the recent mechanistic data have supported a role for Janus kinase-mediated pathways in AA, and several case reports have corroborated that small molecule JAK inhibitors appear to be a promising class of drugs for AA, even in cases of severe or widespread disease.

Second, the molecular definition of AA supports a prominent role for CD8 T cells in the pathogenesis of the human disease. A dose response-like relationship is seen when comparing NC, AAP and AT/AU samples, with progressively increasing gene expression signatures for CD8 T cells, and a supporting peribulbar/perifollicular T cell trend can also be observed. Prior studies have shown that CD8 T cells are necessary and sufficient in a mouse model of AA, and implicated a role for CD8 T cells, by virtue of expression of NKG2D and the association found between AA and NKG2DL, in AA pathogenesis. The data not only further support a role for CD8 T cells in the pathogenesis of disease, but also draws a correlation between the level of CD8 T cell participation and disease severity/phenotype.

Third, the relative similarity between nonlesional and lesional AAP skin samples, compared with the relationship observed between lesional AAP and normal skin samples, suggests that unaffected skin in AA patients share at least some of the factors that predispose skin in AA patients to develop hair loss. However, by virtue of the clinical absence of disease, samples of nonlesional skin do not have the full constellation of factors required for autoimmune destruction of the hair follicle. It seems likely that several immunoregulatory obstacles to breaking tolerance must be overcome in order for the manifestation of clinical disease, with alopecia being observed only when all of these circumstances have occurred. Further examination of the differences between unaffected and affected skin samples from AA patients may be informative as to the final inciting trigger for the development of AA, possibly elucidating new treatment or even preventative strategies in predisposed individuals.

This body of work establishes a molecular definition of the disease process in the skin and may be interrogated for signatures corresponding to protein mediators or cellular participants. These data serve as a rich resource for investigators pursuing pathogenic disease mechanisms and therapeutic targets in AA.

D. Materials and Methods

Human Patient Demographics

Two independent datasets were collected from four National Alopecia Areata Foundation (NAAF) registry sites. The discovery dataset consisted of 81 samples from 63 patients (20 AAP, 20 AT/AU, and 23 Normal controls, with 18 of the AAP also contributing biopsies of nonlesional skin in order to allow for paired comparisons of gene expression between AAP perilesional and nonlesional). The validation dataset was comprised of 41 samples from 33 patients (8 AAP, 12 AT/AU, and 23 Normal controls, with 8 of the AAP patients also contributing biopsies of nonlesional skin in order to allow for paired comparisons of gene expression between AAP lesional and nonlesional samples).

Human Tissue Sampling and Processing

Skin punch biopsy specimens were fixed in the PAXgene Tissue Containers and shipped overnight to Columbia University. Samples were bisected, with one half of the sample processed using the PAXgene tissue miRNA kit to extract RNA and the remaining half embedded in paraffin. Library prep was performed for microarray analysis using Ovation RNA Amplification System V2 and Biotin Encore kits (NuGen Technologies, Inc., San Carlos, Calif.). Samples were subsequently hybridized to Human Genome U133 Plus 2.0 chips (Affymetrix, Santa Clara, Calif.) and scanned at the Columbia University Pathology Core or the Yale Center for Genome Analysis.

Analysis Packages

Quality control of microarrays was performed using the affyAnalysisQC package from http://arrayanalysis.org/. Batch effect correction was Differential expression in these studies was defined by an absolute fold change threshold of 1.5 with a significance threshold of 0.05 Benjamini-Hochberg-corrected. Clustering and principal component analysis was done using the modules provided in the Bioconductor R package. Network images were generated with Cytoscape.

Microarray Preprocessing and Quality Control

Microarray preprocessing was performed using BioConductor in R. Preprocessing of the two datasets, discovery dataset (63 samples) and the validation dataset (33 samples), were performed separately using the same pipeline. Quality control was performed using the affyanalysisQC package from http://arrayanalysis.org/. The discovery dataset and the validation dataset were normalized separately using GCRMA and MASS. The Affymetrix HGU-133Plus2 array contains 54675 probe sets (PSIDs). Filtering was performed so that PSIDs that were on the X or Y chromosome, that were Affymetrix control probe sets, or that did not have Gene Symbol annotation were removed from all arrays for further downstream analysis. For the 3D plot of the ALADIN scores, all 96 samples from both datasets were combined before performing GCRMA normalization and correcting for batch effects.

Sample Filtering and Batch Correction

In order to perform analysis on the 63 AA lesional (both AT/AU and AAP) and NC samples in the discovery data set, PSIDs were further filtered to remove PSIDs that had not been called present on at least one 63 arrays resulting in 36954 PSIDs. Correction for batch effects was performed using the implementation of the function ComBat available in the sva package with gender and AA group (AT/AU, AAP, and normal) used as covariates. No batch correction was required for the validation set. Paired lesional/nonlesional microarrays were processed together within the same microarray batch along with normal controls. The discovery set for the paired lesional/nonlesional analysis was comprised of 18 lesional/nonlesional AAP pairs and 23 controls. The validation set had 8 lesional/nonlesional AAP pairs and 13 NC samples.

In order to examine the relationship between paired nonlesional and nonlesional samples, PSIDs were centered about the mean expression level of the normal samples within each batch. The validation set did not require batch correction.

Differential Expression Analysis

Differential analysis was performed on the batch corrected discovery data set using linear models as implemented in the limma package in Bioconductor. Two-sample comparisons were performed separately to identify PSIDs differentially expressed in AA patients versus normal controls, in AAP patients versus normal controls, and in AT/AU patients versus normal controls treating gender as a fixed factor. Paired comparisons were performed on the AAP-LS/AAP-NL samples treating gender as a fixed effect.

Because the log 2(fold-change) did not exceed 1 for most PSIDs in order to reduce the number of false discoveries retained in the gene expression signature, the inventors sub-sampled the discovery data set leaving samples from one batch out at a time and keeping only those PSIDs that were identified as differentially expressed in the total data set as well as in all subsamplings.

Principal Component Analysis

Principal component analysis was performed on all 36954 PSIDs that were used to perform differential expression analysis. The probability density of the first two principal components was estimated for each group (AT/AU, AAP, and NC) assuming a bivariate distribution. Principal component analysis was performed the 41 AAP-LS, AAP-NL, and normal control samples in the discovery set used the 170 PSIDs that had been identified as differentially expressed.

Histopathological Staining

Immunohistochemistry was performed using the Bond Polymer Refine Red Detection (Leica Biosystems, Buffalo Grove, Ill.) protocol with clone LN10 anti-CD3 primary antibody. The peribulubar/perifollicular histopathological scoring system was conducted using 0-3 scale (0—no immune infiltrate; 1—mild; 2—intermediate; 3—severe) with representative examples shown in FIG. 2C. Using the Kruskal-Wallis test, the null hypothesis that the mean ranks of the CD3 scores were the same in all groups at the significance level of 0.05 was rejected (H=16.51, df=2, p=0.00026). Wilcoxon Rank Sum tests were performed for all pairs comparisons and adjusted for multiple comparisons using a Bonferroni correction. Significant differences were observed between AU/AT and Normal groups (p=4e-04) and AAP and Normal groups (p=0.0062) (wilcox test from the coin package in R using distribution=“asymptotic”).

Generating a Linear Euclidean Classifier for AA Severity

Principal component analysis and terrain mapping of the AA disease signature revealed a near-linear dependency between NC, AAP, and AT/AU patients in an expression space defined by the first two principal components (PCs). The expression terrain map was generated with the MeV software suite using Euclidean distance as a metric and 10 nearest-neighbors as a clustering parameter. In order to convert this into a more intuitive, numeric score predictive of severity, the inventors generated a list of genes that significantly contributed to PC1 and PC2. This was done by rank-sorting the genes' weighted contributions to each PC and selecting the set of genes before the inflection point of the weight distribution. The expression vectors of these genes were then z-score transformed and rank-normalized to generate non-zero, statistically comparable expression values. On a patient-by-patient basis, all genes in each normalized vector were used to construct centroid values in the appropriate PC vector. Each centroid value subsequently corresponds to a cardinal point in a grid defined as PC1×PC2 for each patient. A linear projection was then built between {PC1min×PC2min} and {PC1max×PC2max} and each patient was mapped to this line. The vector was then normalized to bind the values between 0 and 10. Score breakpoints for each cohort (NC 0-2, AAP 2-6, AT/AU 6-10) were obtained by performing a sliding window analysis to identify the score values that maximize the odds ratios of NC and AAP, and AAP and AT/AU falling within each score range.

Generating the Consensus Immune Gene Signatures

Unique signature genes for each of the infiltrate populations were adopted. To generate relative classifiers ranking infiltration, each group of mutually exclusive genes were tested for co-segregation and classification power independently before being integrated into a consensus score for each individual patient. Integration was done in the following steps: z-score transforming all gene vectors to obtain scale-comparable expression values; rank-transforming these vectors to obtain ordered, non-negative values for each gene signal; and finally averaging over the ranks to create a consensus value of the rank-ordered expression for each infiltrating tissue. For the estimation of total infiltrate load per sample, the consensus z-score was transformed back into expression space for each individual gene and normalized to the consensus of housekeeping genes with the minimum coefficient of variation across the population, in a patient-by-patient basis.

The following table shows the signatures for each cell type:

| Cell | Symbol | Entrez | Probe |
|---|---|---|---|
| aDCs | CCL1 | 6346 | 207533_at |
| aDCs | CD83 | 9308 | 204440_at |
| aDCs | LAMP3 | 27074 | 205569_at |
| B-cells | BLK | 640 | 210934_at |
| B-cells | CD19 | 930 | 206398_s_at |
| B-cells | MS4A1 | 931 | 228599_at |
| DCs | CCL13 | 6357 | 216714_at |
| DCs | CCL17 | 6361 | 207900_at |
| DCs | CCL22 | 6367 | 207861_at |
| DCs | CD209 | 3083 | 207278_s_at |
| Eosinophils | CCR3 | 1232 | 208304_at |
| Eosinophils | GPR44 | 1125 | 216464_x_at |
| Eosinophils | IL5RA | 3568 | 211517_s_at |
| iDCs | CD1A | 909 | 210325_at |
| iDCs | CD1E | 913 | 215784_at |
| Macrophages | CCL7 | 6354 | 208075_s_at |
| Macrophages | CXCL5 | 6374 | 215101_s_at |
| Macrophages | FN1 | 2335 | 216442_x_at |
| Macrophages | MSR1 | 4481 | 214770_at |
| Macrophages | PPBP | 5473 | 214146_s_at |
| mast cells | CMA1 | 1215 | 214533_at |
| mast cells | MS4A2 | 2206 | 207497_s_at |
| mast cells | TPSAB1 | 7177 | 216485_s_at |
| Neutrophils | FPRL1 | 2358 | 210773_s_at |
| Neutrophils | IL8RA | 3577 | 207094_at |
| Neutrophils | IL8RB | 3579 | 207008_at |
| NK cells | NCR1 | 9437 | 217095_x_at |
| NK cells | XCL1 | 6375 | 206366_x_at |
| Normal | DCN | 1634 | 242605_at |
| pDC | CLEC4C | 1704 | 1555687 a_at |
| T-cells | CD2 | 914 | 205831_at |
| T-cells | CD247 | 919 | 210031_at |
| T-cells | CD28 | 940 | 211861_x_at |
| T-cells | CD3E | 916 | 205456_at |
| T-cells | CD3G | 917 | 206804_at |
| T-cells | CD6 | 923 | 213958_at |
| T-cells | IL2RB | 3560 | 205291_at |
| T-cells | ZAP70 | 7535 | 214032_at |
| CD8 T-cells | CD8A | 925 | 205758_at |
| CD8 T-cells | PRF1 | 5551 | 214617_at |
| Cytotoxic cells | KLRF1 | 5134 | 220646_s_at |
| Cytotoxic cells | GNLY | 1057 | 37145_at |
| Cytotoxic cells | GZMA | 3001 | 205488_at |
| Cytotoxic cells | GZMH | 2999 | 210321_at |
| Cytotoxic cells | GZMK | 3003 | 206666_at |
| Tem | LTK | 4058 | 217184_s_at |
| Tem | NFATC4 | 4776 | 236270_at |

Unsupervised Machine Learning, Weighted Gene Co-Expression Analysis

Weighted Gene Co-expression Analysis (WGCNA) was performed on the PSIDs in the ComBat adjusted expression set whose variance exceeded the median of the variances of all the PSIDs. Adjacency between PSIDs was defined as the Pearson correlation of the expression profiles across samples raised to a soft-thresholding power equal which was set to 10. The resulting adjacency matrix was transformed into a Topological Overlap matrix (TOM) from which the dissimilarity matrix was calculated. Hierarchical clustering was performed on the dissimilarity matrix and modules were identified from the resulting branches of the dendrogram. The coexpression heatmap and dendrogram in FIG. 5 were created from stratified sampling of ⅓ of the PSIDs assigned to the 20 modules using TOM as the adjacency measure. Kruskal-Wallis tests were performed to test for association between module eigengenes and the categorical traits disease phenotype, gender, and originating NAAF site. Pearson's correlation coefficient and p-values were estimated between each module eigengene and subject age. Gene Set Enrichment Analysis (GSEA) was used to further test for overrepresentation of genes over/under expressed in AA with respect to Normal controls in the different WGCNA modules. The normalized enrichment score (NES) reflects the degree to which a gene set is overrepresented at the top or bottom of a ranked list of genes taking into account differences in module size. Preranked gene lists were created with the t-statistic for ranking.

Calculation of ALADIN Scores

The CTL, IFN and KRT ALADIN scores were calculated for each sample. Briefly, z-scores are calculated for each PSID relative to the mean and standard deviation of normal controls. Z-scores for each gene are obtained by averaging z-scores of PSIDs mapping to that gene. Signature scores are then calculated averages of the z-scores for genes belonging to the corresponding signature.

### 6.2 Example 2

A. Introduction

AA is a T cell-mediated autoimmune disease characterized phenotypically by hair loss and, histologically, by infiltrating T cells surrounding the hair follicle bulb. Transfer of total T cells (but not B cells or sera) can cause the disease in human xenograft models, as well as in C3H/I-IeJ mice, a mouse strain that develops spontaneous AA with considerable similarity to human AA. Broad-acting intralesional steroids are the most commonly used therapy for AA, with varying success. Progress in developing effective, rationally targeted therapies has been limited by the lack of mechanistic understanding of the underlying key T cell inflammatory pathways in AA.

A cytotoxic subset of CD8+NKG2D+ T cells was identified within the infiltrate surrounding human AA hair follicles. Also identified was concomitant upregulation in the follicle itself of the ‘danger signals’ UI 103 and MICA, two NKG2D ligands (NKG2DLs) whose importance in disease pathogenesis has also been suggested by genome-wide association studies.

B. Results

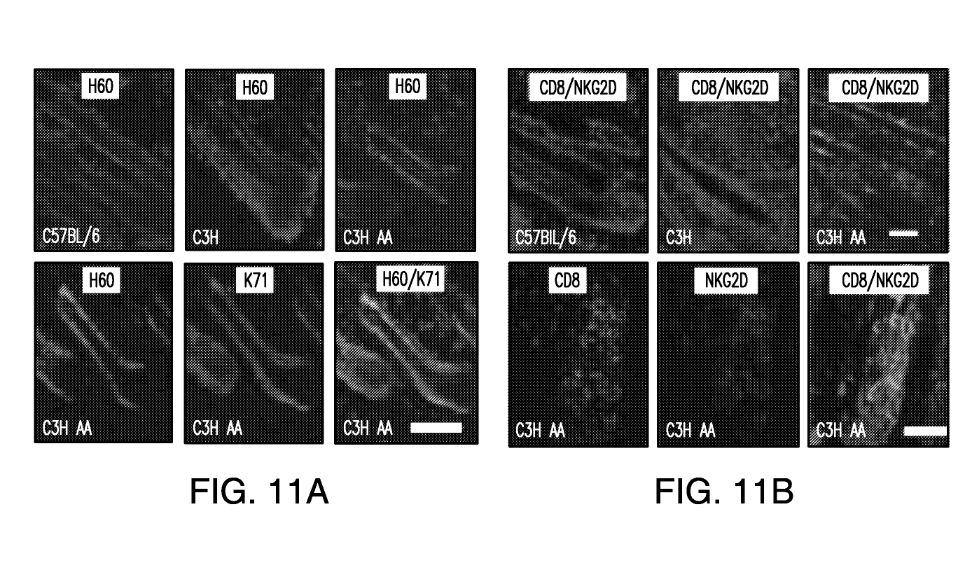

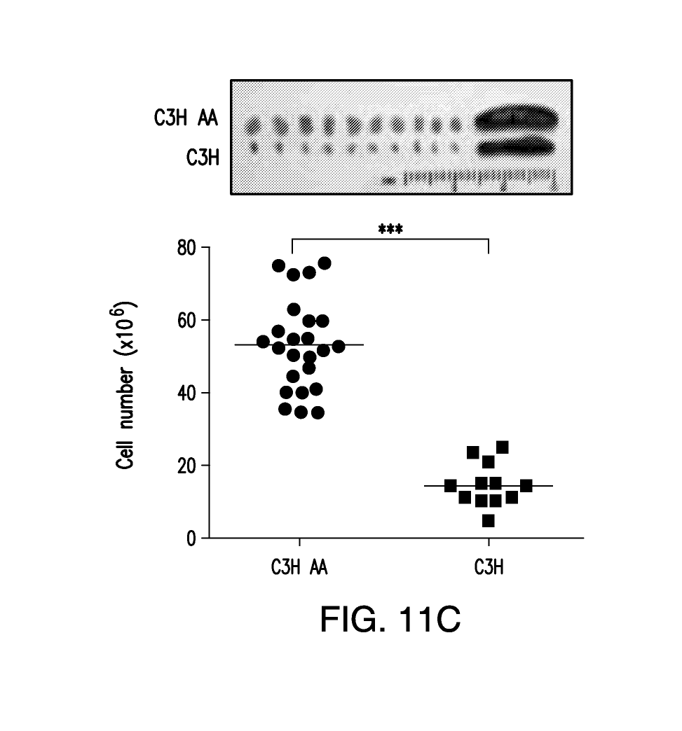

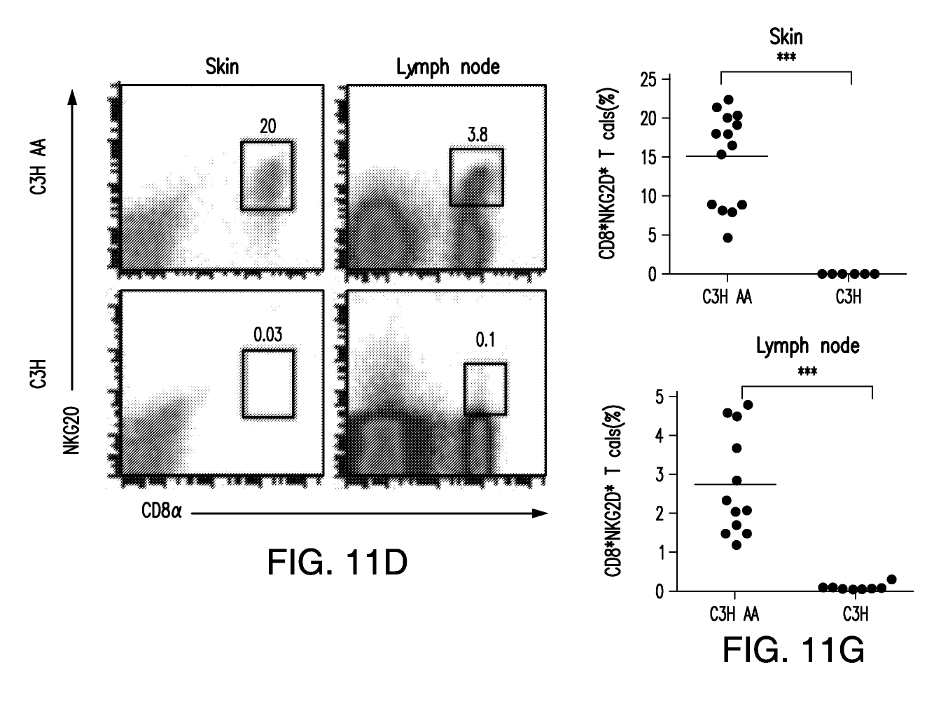

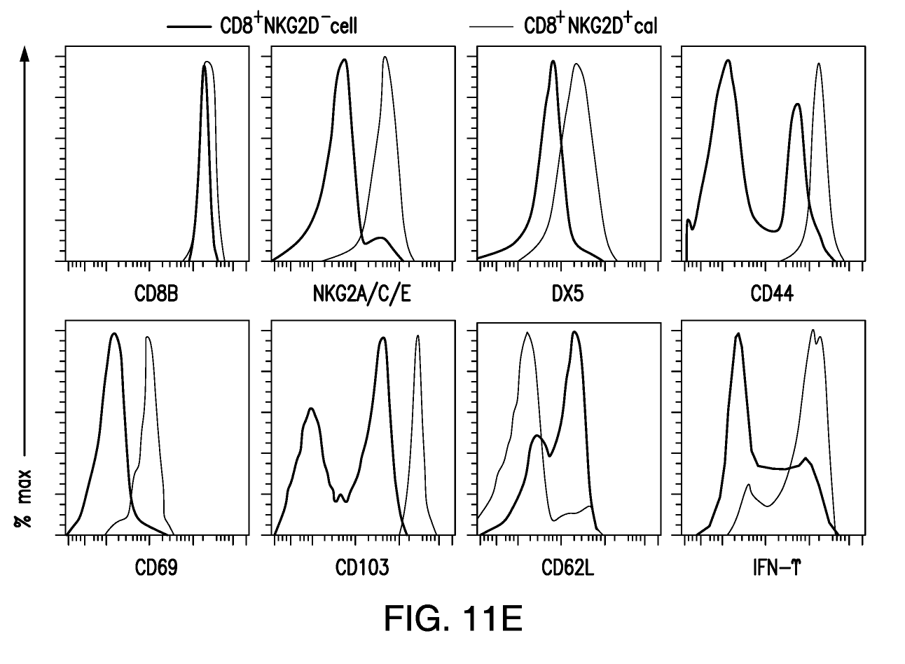

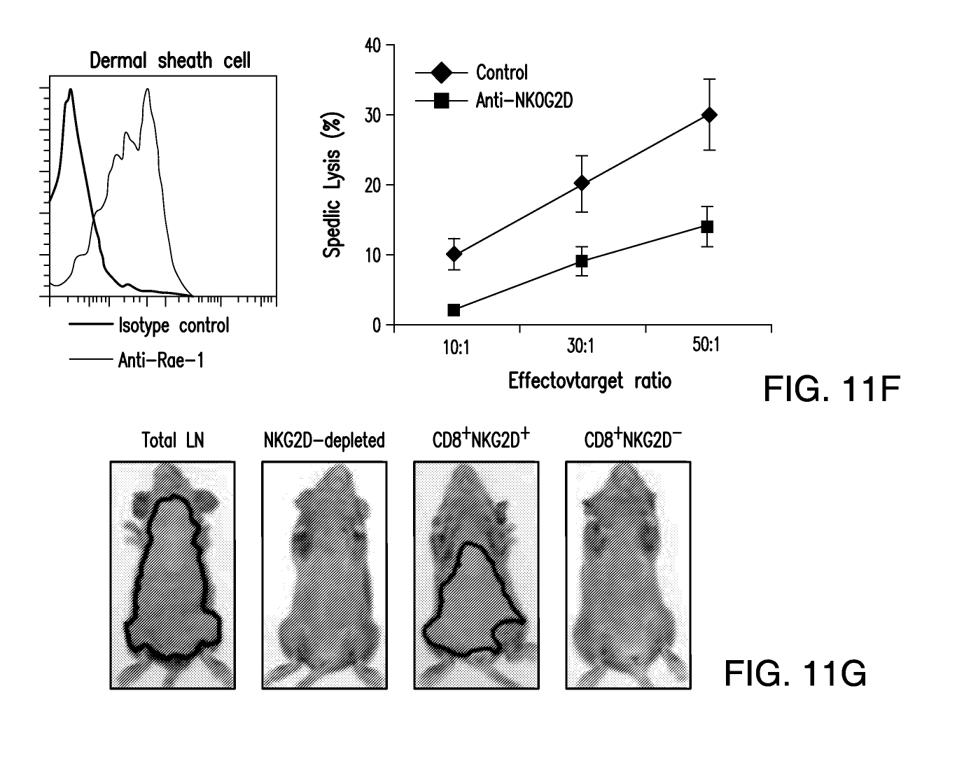

To determine the contribution of CD8-i-NKG2D+ T cells to AA pathogenesis, the inventors used the C3H/HeJ mouse model, which spontaneously develops alopecia and recapitulates many pathologic features of human AA. In lesional skin biopsies from alopecic mice, CD8+NKG2D+ T cells infiltrate the epithelial layers of the hair follicle, which overexpress the NKG2DLs, H60 and Rae-1, analogous to what has been observed in skin biopsies of human AA (FIG. 11A-11B). Flow cytometric analysis of the CD45+ leukocyte population in the skin revealed a marked increased number of CD8+NKG2D+ T cells in the skin of diseased C3H/HeJ mice, in conjunction with cutaneous lymphadenopathy and increased total cellularity, as compared with disease-free C3H/HeJ mice (FIG. 11C-11D). Other cell types, including CD4+ T cells4 and mast cells, were present in much smaller numbers.

The immunophenotype of the skin-infiltrating CD8+ T cells in mice with AA was similar to that of the CD8+NKG2D+ population found in the cutaneous lymph nodes: CD8αβ+ effector memory T cells (TEM, CD8hiCD44hiCD62LlowCD103+) bearing several natural killer (NK) immunoreceptors, including CD49b and NKG2A, NKG2C and NKG2E (FIG. 11E). These CD8+ TEM cells expressed high levels of IFN-γ and exhibited NKG2D-dependent cytotoxicity against ex vivo-expanded syngeneic dermal sheath target cells (FIG. 11.F), Gene expression analysis of the CD8+NKG2D T cells isolated from alopecic C3H/HeJ lymph node cells using RNA-seq demonstrated a transcriptional profile characteristic of effector cytotoxic T lymphocytes (CTLs), and identified several additional NK-specific transcripts.

The inventors next evaluated the requirement of these CD8+ TEM cells in disease pathogenesis. Transfer of cytotoxic CD8+NKG2D+ cells or total lymph node cells from diseased mice induced AA in all five healthy C3H/HeJ recipients by 14 weeks after transfer, whereas lymph node cell populations depleted of NKG2D+ cells were unable to transfer disease (FIG. 11G). Thus, CD8+NKG2D+ T cells are the dominant cell type in the dermal infiltrate and are necessary and sufficient for T cell-mediated transfer of AA.

To characterize the transcriptional profile of AA lesional skin from C3H/HeJ mice as well as human AA, the inventors performed Affymetrix microarray analyses to identify differentially expressed genes in skin between individuals with AA and skin from control individuals without disease. Three gene expression signatures were identified in lesional skin: IFN response genes, such as those encoding the IFN-inducible chemokines CXCL-9, CXCL-10 and CXCL-11, several key CTL-specific transcripts, such as those encoding CD8A and granzymes A and B, and γ c cytokines and their receptors, such as the transcripts for interleukin-2 (IL-2) and IL-15, in both human and mouse AA skin. As IL-2Rα was previously shown to be expressed on infiltrating lymphocytes surrounding human AA hair follicles, the inventors performed immunofluorescence analysis for both IL-15 and its chaperone receptor IL-15Rα to identify the source of IL-15 in the skin. The inventors detected a marked upregulation of both components in AA hair follicles in both human and mouse AA and found IL-15Rβ expressed on infiltrating CD8+ T cells in humans.

IL-2 and IL-15 are well-known drivers of cytotoxic activity by IFN-γ-producing CD8+ effector T cells and NK cells and have been implicated in the induction and/or maintenance of autoreactive CD8+ T cells. To test the efficacy of IFN-γ- and γ c-targeted therapies in vivo, the inventors used the well-established graft model of AA, in which skin grafts from mice with spontaneous AA are transferred onto the backs of unaffected 10-week-old recipient C3H/HeJ mice. In this model, AA develops reliably in 95-100% of grafted recipients within 6-10 weeks, allowing us to test interventions aimed at either preventing or reversing disease.

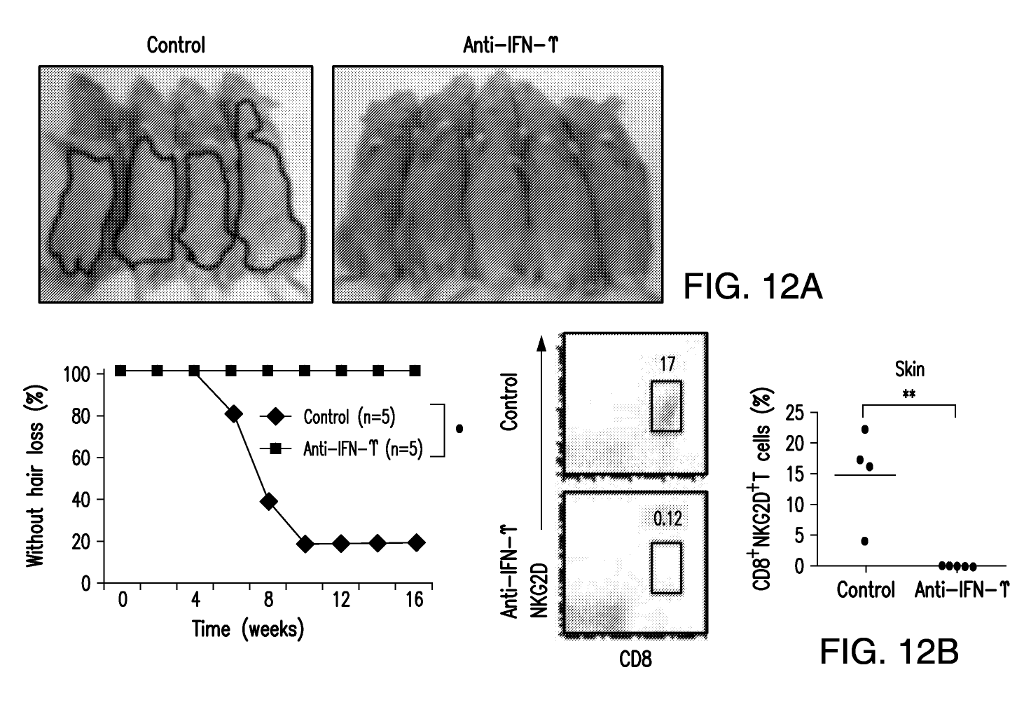

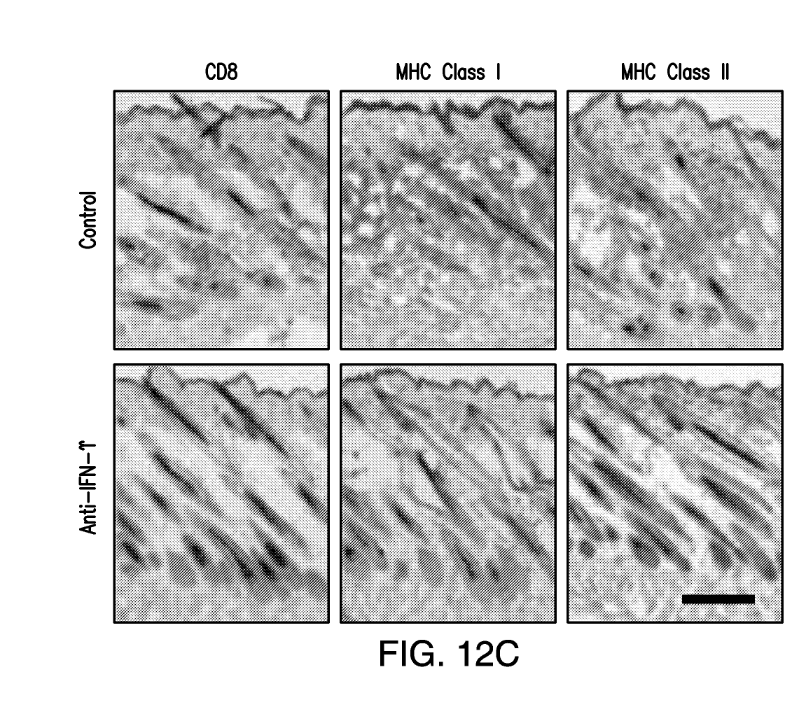

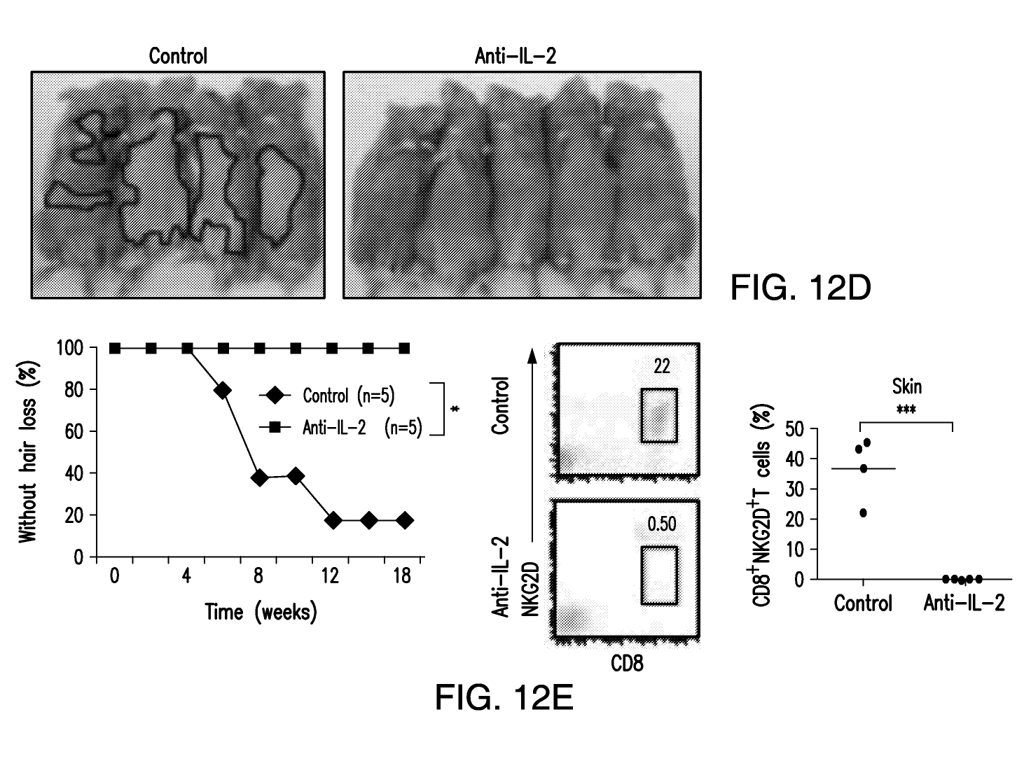

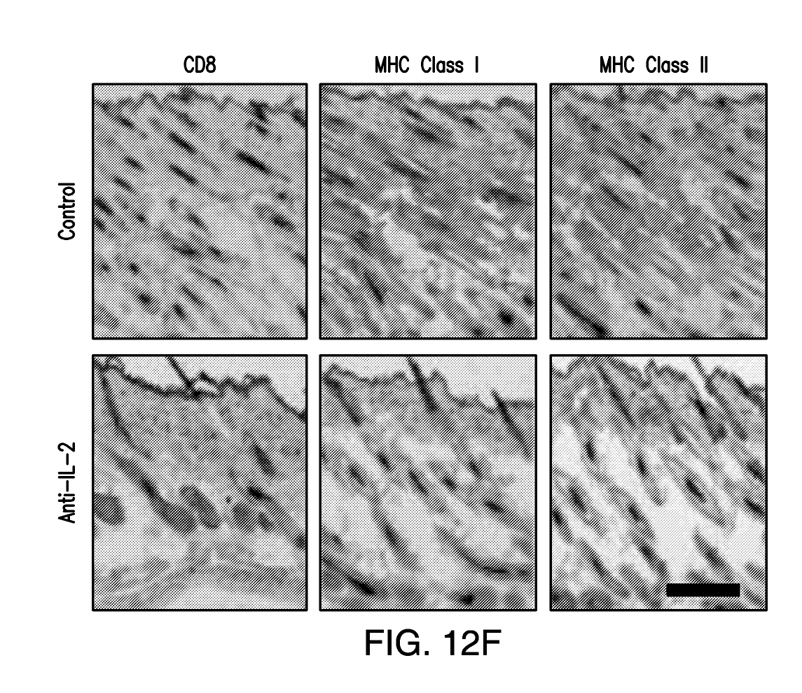

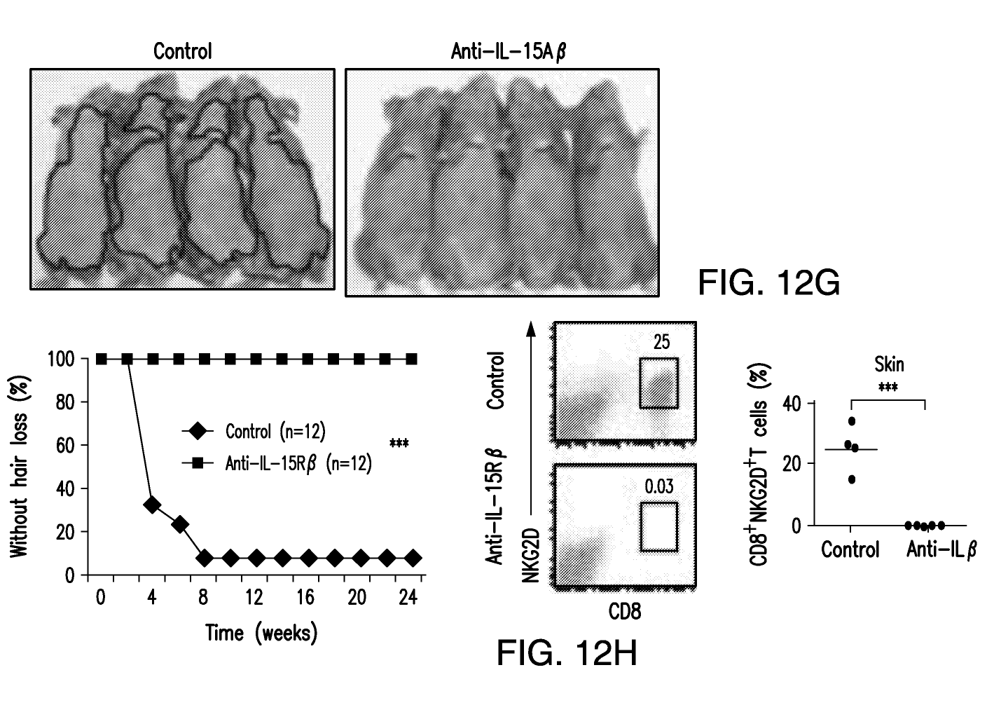

The role of IFN-γ in AA was previously investigated using both knockout studies and administration of IFN-γ, where IFN-γ-deficient mice were resistant and exogenous IFN-γ precipitated disease. Administration of neutralizing antibodies to IFN-γ at the time of grafting prevented AA development in grafted recipients and abrogated major histocompatibility complex (MHC) upregulation and CD8+NKG2D+ infiltration in the skin (FIG. 12A-12C). Likewise, a role for IL-2 in AA pathogenesis was previously established using genetic experiments in which IL-2 haploinsufficiency on the C3H/HeJ background conferred resistance to disease by about 50% using the graft model, and this role is supported by the genome-wide association studies in humans. Systemically administered blocking antibodies to either IL-2 (FIG. 12D-12F) or IL-15Rβ (FIG. 12G-12I) prevented AA in grafted mice, blocked the accumulation of CD8+NKG2D+ T cells in the skin and abrogated MHC upregulation. However, IL-21 blockade failed to prevent the development of AA in grafted C3H/HeJ mice Notably, none of these blocking antibodies given alone was able to reverse established AA (data not shown).

The inventors next asked whether the inventors could recapitulate the effects of type I cytokine blockade by intervening downstream using small-molecule inhibitors of JAK kinases, which signal downstream of a wide range of cell surface receptors. In particular, IFN-γ receptors and γ c family receptors signal through JAK1/2 and JAK1/3, respectively. JAK activation was shown by the presence of phosphorylated signal transducer and activator of transcription (STAT) proteins (pSTAT1, pSTAT3 and to a lesser extent pSTAT5) in human and mouse alopecic hair follicles, but not in normal hair follicles. In in vitro-cultured dermal sheath cells from C3H/HeJ mice, exogenous IFN-γ increased STAT1 activation, whereas IFN-γ plus TNF-α increased surface IL-15 expression. Ruxolitinib, a US Food and Drug Administration (FDA)-approved small-molecule inhibitor of the JAK1/2 kinases (JAK selectivity is JAK1=JAK2>Tyk2>>>JAK3) critical for IFN-γ R signaling inhibited these responses. In cultured CTL effectors from C3H/HeJ mice, the FDA-approved small-molecule JAK3 inhibitor tofacitinib (JAK3>JAK1>>JAK2 selectivity) blocked IL-15-triggered pSTAT5 activation. Tofacitinib also blocked killing of dermal sheath cells and IL-15-induced upregulation of granzyme B and IFN-γ expression.

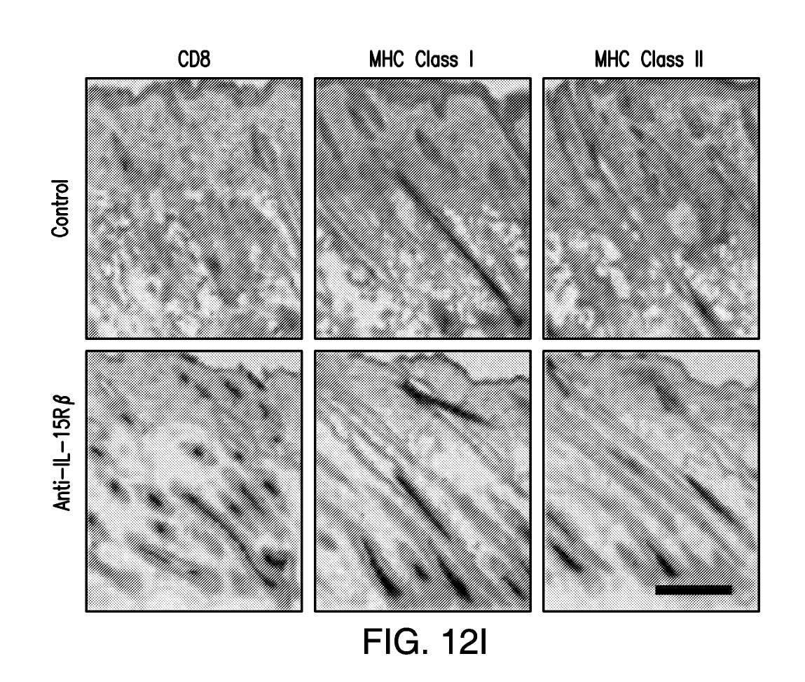

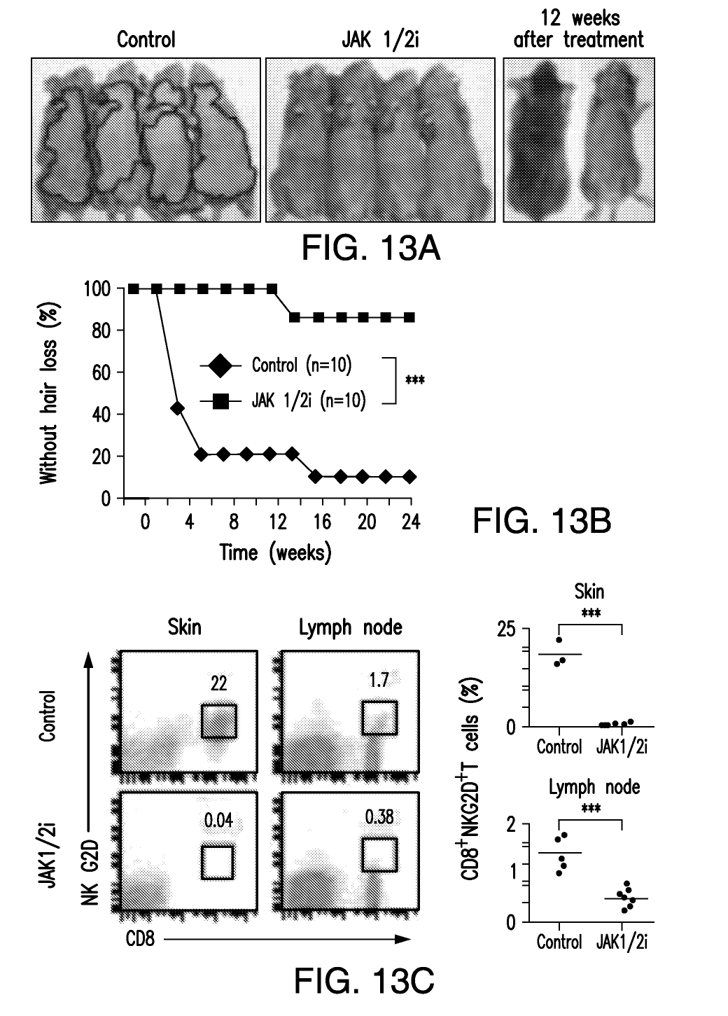

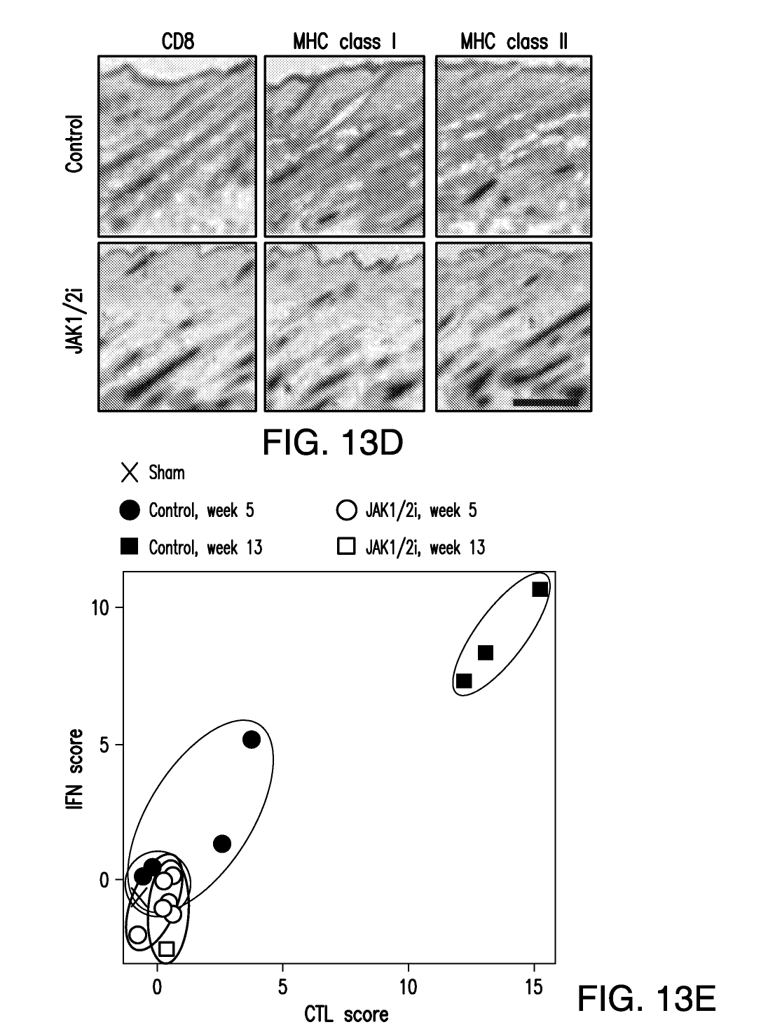

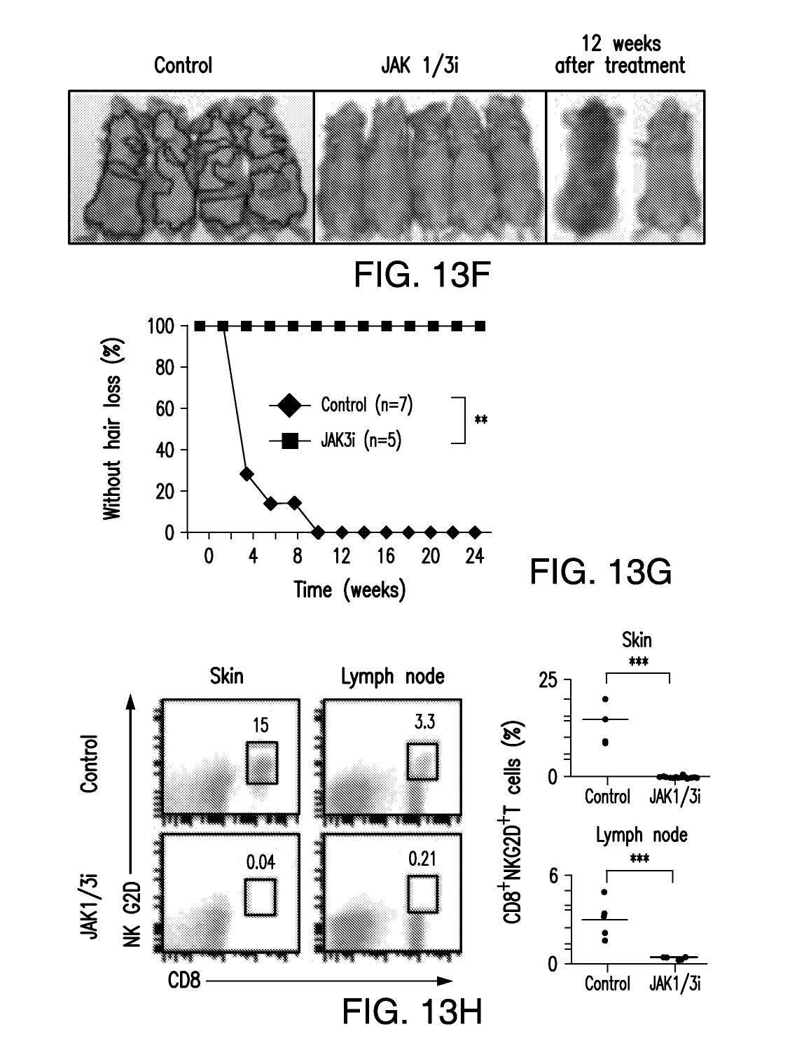

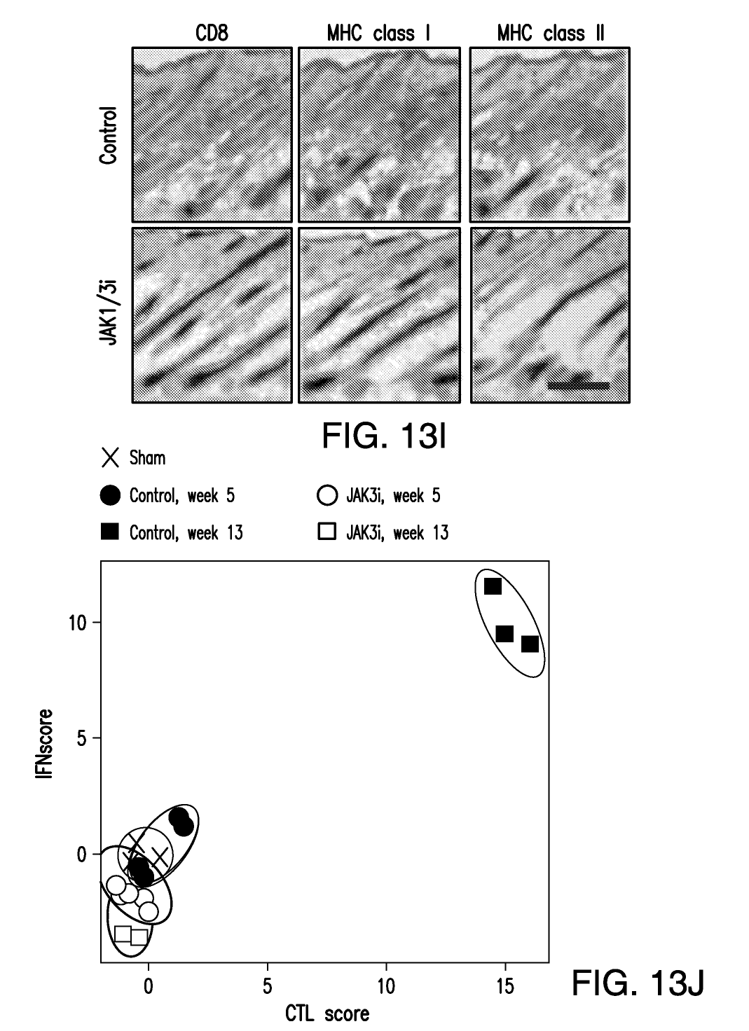

To test whether inhibition of these signaling pathways would be therapeutically effective in vivo, the inventors systemically administered ruxolitinib (FIG. 13A-13C) and tofacitinib (FIG. 13F-13H) at the time of grafting and found that they prevented the development of AA and the expansion of CD8+NKG2D+ T cells in all grafted recipients.

The skin of mice treated with either drug showed no histological signs of inflammation (FIGS. 13D & 13I). Global transcriptional analysis of whole-skin biopsies showed that both drugs also blocked the dermal inflammatory signature, as measured by Alopecia Areata Disease Activity Index (ALADIN, FIGS. 13E & 13J), and Gene Expression Dynamic Index (GEDI) analysis.

The inventors next asked whether systemic tofacitinib treatment could reverse established disease by initiating therapy 7 weeks after grafting, a time point at which all mice had developed extensive AA. Systemic therapy resulted in substantial hair regrowth all over the body, reduced the frequency of CD8+NKG2D+ T cells and reversed histological markers of, all of which persisted 2-3 months after the cessation of treatment.

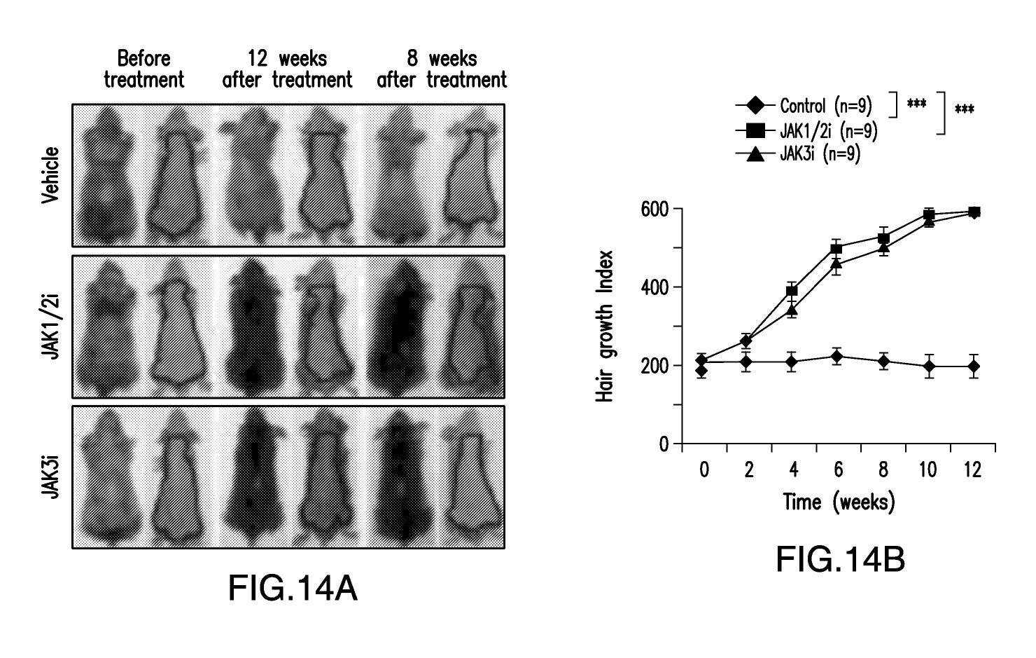

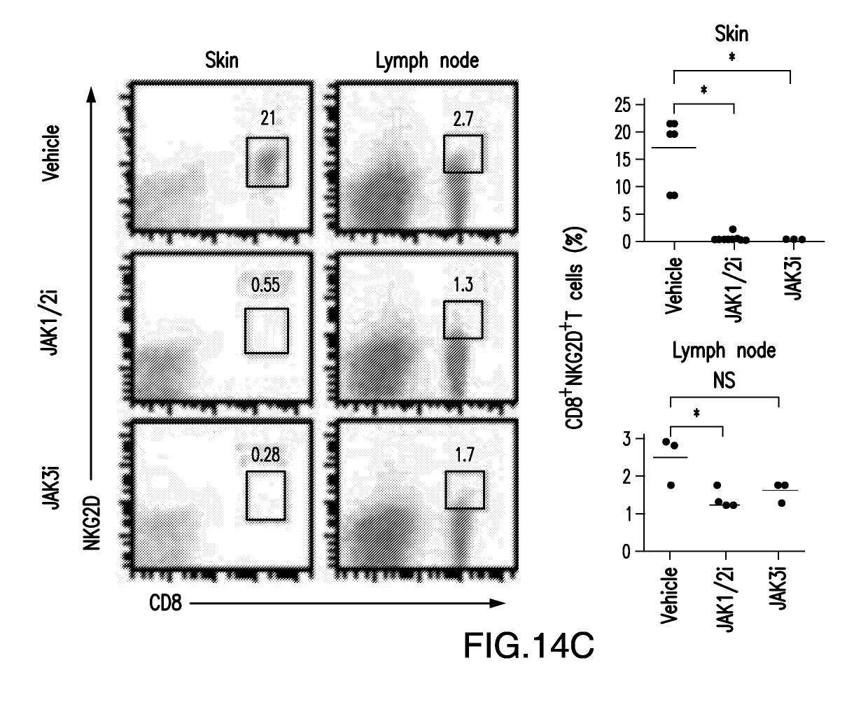

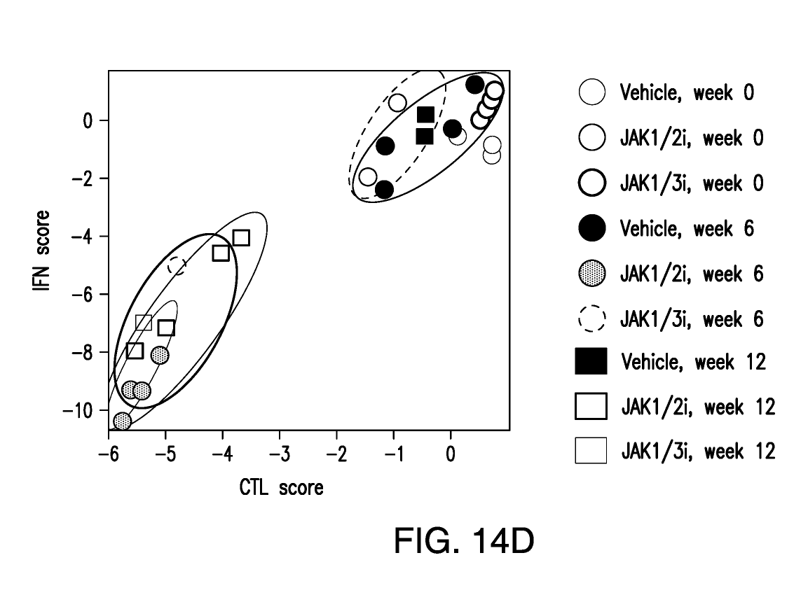

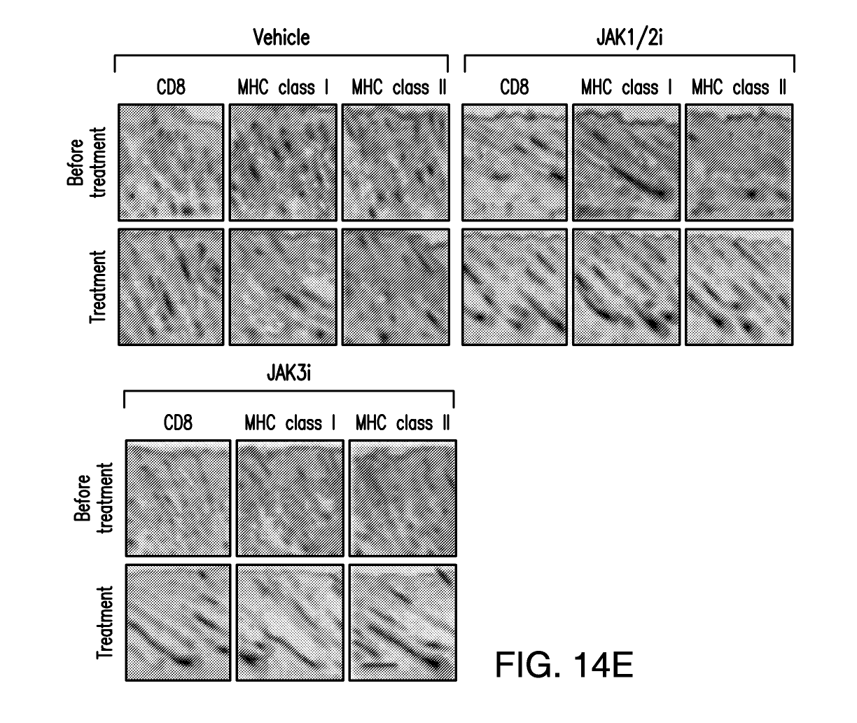

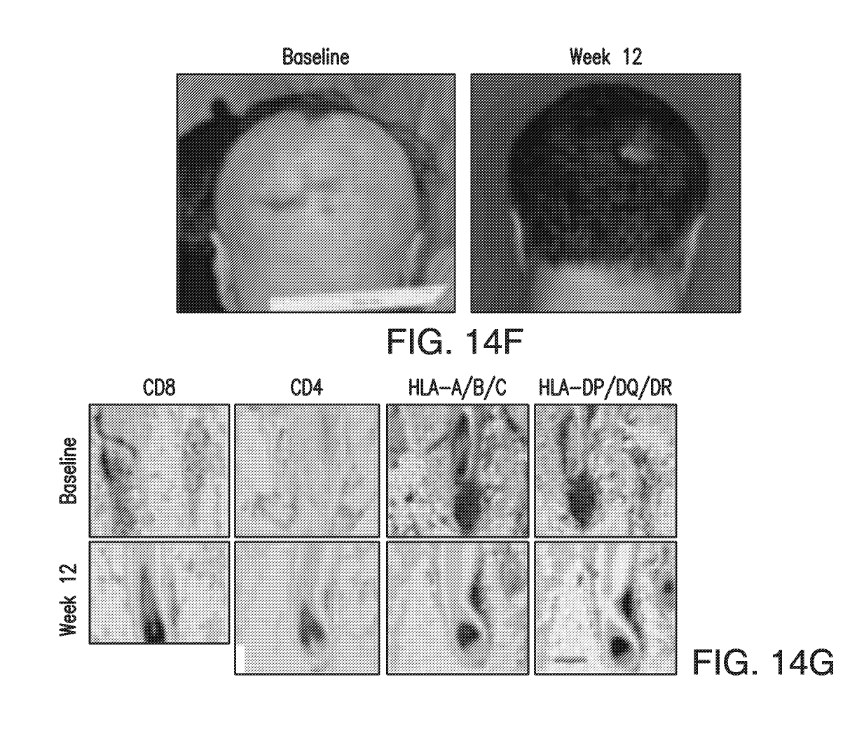

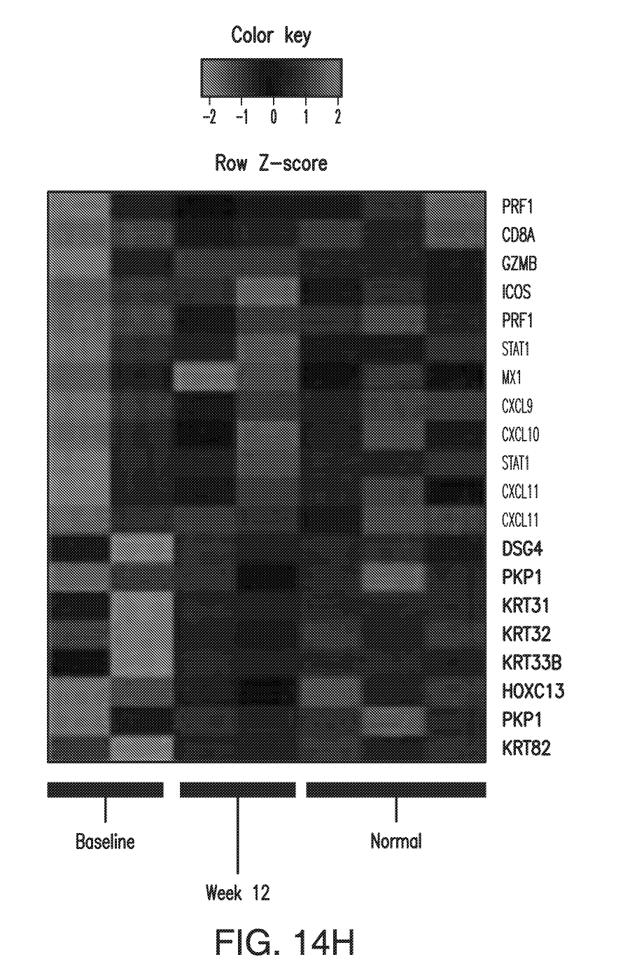

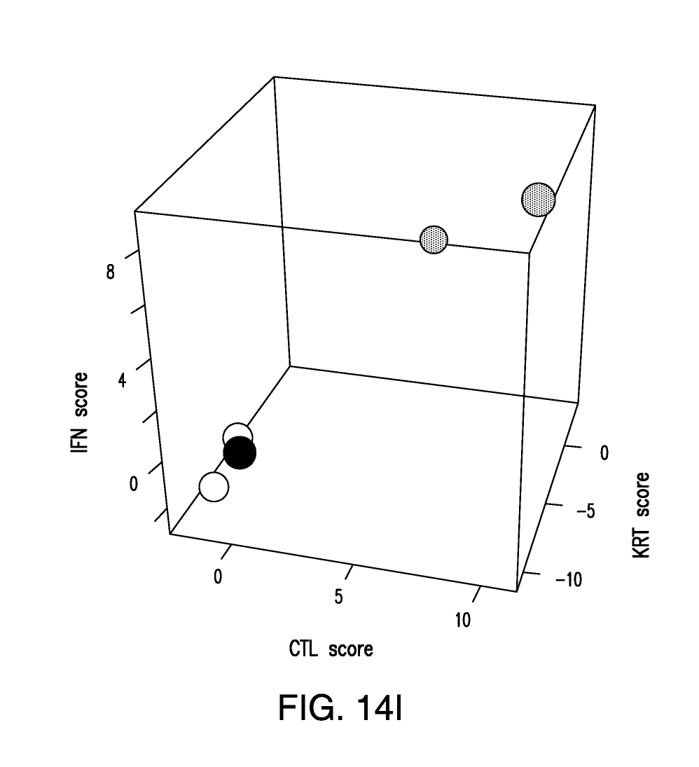

Next, to test a more clinically relevant route of delivery, the inventors asked whether topical administration of protein tyrosine kinase inhibitors could reverse established AA in mice with kinetics similar to those of systemic delivery. In established disease, the inventors found that topical ruxolitinib and topical tofacitinib were both highly effective in reversing disease in treated lesions (applied to back skin). A full coat of hair emerged in the ruxolitinib- or tofacitinib-treated mice by 7 weeks of treatment, and the inventors observed complete hair regrowth within 12 weeks following topical therapy (FIG. 14A-14B). Topical therapy was associated with a markedly reduced proportion of CD8+NKG2D+ T cells in the treated skin and lymph node (FIG. 14C), normalization of the ALADIN transcriptional signature (FIG. 14D), reversal of histological markers of disease (FIG. 14E) and correction of the GEDI in all treated mice. Notably, untreated areas on the abdomen remained alopecic (e.g., FIG. 14A), demonstrating that topical therapy acted locally and that the observed therapeutic effects were not the result of systemic absorption. These effects were visible as early as 2-4 weeks after the onset of treatment and persisted 2-3 months after the cessation of treatment (FIG. 14A).

To test the efficacy of JAK inhibitors in human subjects with AA, the inventors treated three patients with moderate to severe disease orally with ruxolitinib, 20 mg twice daily. Ruxolitinib is currently FDA-approved for the treatment of myelofibrosis, a disease driven by wild-type and mutant JAK2 signaling downstream of hematopoietic growth factor receptors. In addition, small clinical studies using topical ruxolitinib in psoriasis have demonstrated anti-inflammatory activity that may be due to interruption of the IL-17 signaling axis. All three ruxolitinib-treated patients exhibited near-complete hair regrowth within 3 to 5 months of oral treatment (e.g., FIG. 14F). Comparison of biopsies obtained at baseline and after 12 weeks of treatment demonstrated reduced perifollicular T cell infiltration, reduced follicular expression of human leukocyte antigen class I and class II expression (FIG. 14G) and normalization of the ALADIN inflammatory and hair keratin signatures following treatment (FIGS. 14H & 14I).

C. Discussion

Taken together, the data suggest CD8+NKG2D+ T cells promote AA pathogenesis, acting as cytolytic effectors responsible for autoimmune attack of the hair follicle. The inventors postulate that IFN-γ produced by CD8 T cells leads to the collapse of immune privilege in the hair follicle, inducing further production of IL-15 and a feed-forward loop that promotes type I cellular autoimmunity. The clinical response of a small number of patients with AA to treatment with the JAK1/2 inhibitor ruxolitinib suggests future clinical evaluation of this compound or other JAK protein tyrosine kinase inhibitors currently in clinical development is warranted in AA.

D. Materials and Methods

Mice

C3H/HeJ mouse strain (Jackson Laboratories, Bar Harbor, Me.) was used for all animal studies. Only female mice were used. Mouse recipients of alopecic skin grafts were aged 7-10 weeks at the time of grafting. For prevention experiments, drug administration began the day after grafting. For systemic treatment studies, drug administration was initiated approximately 3 months after mice lost their hair. For topical treatment studies, drug administration was initiated 20 weeks following grafting. All animal procedures were done according to protocols approved by the Columbia University Medical Center Institutional Animal Care and Use Committee.

Human Studies

All human studies have been approved by the Columbia University Medical Center Institutional Review Board and were conducted under the Declaration of Helsinki principles. Informed written consent was received from participants before inclusion in the study.

Clinical Evaluation of Oral Ruxolitinib in Alopecia Areata

The inventors initiated a single center, proof-of-concept clinical trial in the Clinical Trials Unit in the Department of Dermatology at the Columbia University Medical Center entitled “An Open-Label Pilot Study to Evaluate the Efficacy of RUXOLITINIB in Moderate to Severe Alopecia Areata” (clinicaltrials.gov identifier: NCT01950780).

The primary efficacy endpoint of this initial pilot study is the proportion of responders achieving 50% or greater regrowth at the end of treatment compared to baseline. Secondary endpoints include the changes in hair growth both during and after treatment measured as a continuous variable; patient global assessments; quality of life assessments; and durability of response following treatment cessation.

Inclusion criteria included 30 to 95% hair loss due to alopecia areata (AA) as measured by SALT score; hair loss duration of at least 3 months; stable hair loss without active evidence of regrowth; subject age 18-75 years.

Exclusion criteria included active scalp disease other than AA; medical history that might increase the risks related to ruxolitinib e.g. hematologic, infectious, immune related diseases or malignancies; current treatment with any modality that might affect AA response; medications known to interact with ruxolitinib; pregnancy; etc.

Subjects on study are treated with oral ruxolitinib 20 mg BID for at least 3 months. The patients in this manuscript have achieved over 90% regrowth. Skin punch biopsies (4 mm) were obtained at baseline and after 12 weeks of treatment.

Antibodies Used for Mice Treatment, Flow Cytometry, Immunostaining and Western Blot Analysis

All antibodies used in these studies are listed in table form below.

Flow cytometric analysis used the following anti-mouse antibodies: CD3 (17A2, Ebioscience), CD4 (GK1.5, BD), CD8α (53-6.7, BD), CD8β (YTS156.7.7, Biolegend), NKG2D (CX5, Ebioscience), NKG2A/C/E (clone 20d5, Ebioscience), CD44 (IM7, BD), CD45 (30-F11, BD), CD49b (Dx5, BD), CD62L (MEL-14, BD), CD69 (H1.2F3, BD), CD103 (2E7, eBioscience), IFNγ (XMG1.2, Ebioscience), Granzyme B (NGZB, eBioscience), Rae-1 (186107, R&D).

For immunohistochemical studies of mouse skin, 8 μM methanol-fixed frozen skin sections were stained with primary rat antibodies (Biolegend) including: anti-CD8 (clone 53-6.7), Biotin anti-MHC class I (clone 36-7.5), anti-MHC class II (clone M5/114.15.2). Biotinylated goat anti-rat IgG (Life Technologies) was used as secondary antibody. For immunofluorescence studies anti-H60 (R&D, clone 205326), anti-Pan Rae-1 (R&D, clone 186107), anti-NKG2D (R&D clone 191004), anti-IL-15 (SCBT, H-114), anti-IL-15 RA (SCBT, N-19), anti-K71 (Abcam), primary antibody were used in immunofluorescence. Alexa Fluor 488 or Alexa Fluor 594-conjugated goat anti-Rat, donkey anti-Rabbit or donkey anti-Goat antibody was used as secondary antibody (Life Technologies).

For immunohistochemical studies of human skin, 5 μM formalin fixed and paraffin skin section were used. After heat antigen retrieval, skin sections were stained with primary anti-human antibodies including: anti-CD8(Abcam ab4055), anti-CD4, (Leica clone 1-F6), HLA Class 1 ABC(Abcam clone EMR8-5), HLA-DR/DP/DQ(SCBT clone CR3/43). ImmPRESS HRP Anti Rabbit Ig or Mouse Ig (Peroxidase) Polymer (Vector Lab) were used as secondary antibody.

Human hair follicles were microdissected and embedded in OCT compound prior to sectioning and staining. 8 μM methanol-fixed frozen sections were stained with anti-IL-15 (SCBT, H-114) and anti-IL-15 RA (SCBT, N-19) or anti-IL-15 RB (SCBT, C-20) and CD8 (SCBT, C8/144B) followed by staining with Alexa Fluor 488 or Alexa Fluor 594-conjugated secondary antibody (Life Technologies). All images were captured with an SDRC Zeiss Exciter Confocal Microscope.

For western blotting, samples with treatment were resolved by 4-12% SDSPAGE (Life Technologies) and then transferred to Westran PVDF membranes (GE Healthcare life Sciences). Blots were probed with the following Abs (All from Cell Signaling Technology): anti-phospho STAT1 (Tyr701), anti-phospho-STAT5 (Tyr694), anti-STAT1 and anti-STAT5.

Antibodies for In Vivo Treatment

| Mouse antibody | Company | Clone | Cat No. |
|---|---|---|---|
| Anti-IL15 Rβ | Biolegend | TM-β1 | 123204 |
| IL-2 | BioXcel | S4B6-1 | BE0043-1 |
| IL-2 | BioXcel | JES6-1A12 | BE0043 |
| IFN-γ | BioXcel | H22 | BE0254 |
| IL-21 | Ebioscience | FFA21 | 16-7211-85 |

Antibodies for Flow Cytometry (1/100 Dilution)

| Mouse | | | |
|---|---|---|---|
| antibody | Company | Clone | Cat No. |
| CD3 | Ebioscience | 17A2 | 17-0032 |
| CD4 | BD | GK1.5 | 560181 |
| CD8a | BD | 53-6.7 | 560469 |
| CD8b | Biolegend | YTS156.7.7 | 126610 |
| NKG2D | Ebioscience | CX5 | 12-5882 |
| NKG2A/C/E | Ebioscience | 20d5 | 13-5896 |
| CD44 | BD | IM7 | 553133 |
| CD45 | BD | 30-F11 | 552848 |
| D49b | BD | Dx5 | 553857 |
| Cd62L | BD | MEL-14 | 553152 |
| CD69 | BD | H1.2F3 | 557392 |
| CD103 | Ebioscience | 2E7 | 17-1031 |
| IFNγ | Ebioscience | XMG1.2 | 11-7311 |
| Pan Rae-1 | R&D systems | 186107 | MAB17582 |
| Granzyme B | Ebioscience | NGZB | 11-8898 |

Antibodies for Immunostaining and Western Blot (1/100 Dilution Unless Otherwise Noted)

| Human antibody | Company | Clone | Cat No. |
|---|---|---|---|
| CD3 | Abcam | PS1 | Ab699 |
| CD8 | SCBT | C8/1448 | Sc-53212 |
| CD4 | Leica | 1-F6 | CD4-1F6-L-CE |
| HLA Class I ABC | Abcam | EMR8-5 | ab70328 |
| HLA-DR/DP/DQ | SCBT | CR3/43 | sc-53302 |

| Mouse antibody | Company | Clone | Cat No. |
|---|---|---|---|
| CD8 | Biolegend | 53-6.7 | 100702 |
| MHC-class I | Biolegend | 36-7.5 | 114903 |
| MHC-class II | Biolegend | M5/114.15.2 | 107602 |
| H60 | R&D systems | 205326 | MAB1155 |
| Pan Rae-1 | R&D systems | 186107 | MAB17582 |
| NKG2D | R&D systems | 191004 | MAB1547 |

| | | | | IF/IHC |
|---|---|---|---|---|
| Mouse/Human antibody | Company | Clone | Cat No. | Dilution |
| IL-15 | SCBT | polyclonal | sc-7889 | |
| | | H-114 | | |
| IL-15RA | SCBT | polyclonal | sc-1524 | |
| | | N-19 | | |
| Phospho-Stat1 (Tyr701) | Cell signaling | D4A7 | 7649 | |
| Phospho-Stat3 (Tyr705) | Cell signaling | D3A7 | 9145 | 1/200 |
| Phospho-Stat5 (Tyr694) | Cell signaling | C11C5 | 9359 | 1/400 |
| Stat1 | Cell signaling | polyclonal | 9172 | |
| Stat5 | Cell signaling | polyclonal | 9363 | |
| K71 | Abcam | polyclonal | Ab133817 | |

STAT1, STAT5, pSTAT1 and pSTAT5 ab's were diluted 1/1000 for western blots.

IL-15 and IL-15RA Staining Blocking Reagents

| Blocking reagent | Company | Cat No. |
|---|---|---|
| IL-15 | Peprotech | AF-200-15 |
| IL-15 RA blocking peptide | SCBT | sc-1524 P |

RNA-Seq Analysis

Samples were sequenced on the HiSeq 2000 sequencer (Illumina, San Diego, Calif.) for 50 cycles. RNA-Seq files were demultiplexed by the Rockefeller University Genomics Core Facility. Quality control of the sample fastq files was performed using fastqc. TopHat was used to map transcripts to the UCSC mm9 reference genome from iGenome. The RefSeq gene annotation packaged with this iGenome version of the UCSC mm9 were used. The htseq-count utility from the HTSeq package was used to convert TopHat bam files to counts that could be used as input for downstream analysis of differential expression with edgeR. Absent genes were removed and a pseudocount of 1 was added in order to avoid division by zero in downstream analysis. EdgeR was used to identify differentially expressed genes using a matched pairs design with three biological replicates.

Microarray Analysis

Quality Control, Preprocessing

For the mouse cDNA samples were hybridized to the Mouse Genome 430 2.0 gene chips and subsequently washed, stained with streptavidin-phycoerythrin, and scanned on an HP GeneArray Scanner (Hewlett-Packard Company, Palo Alto, Calif.). For the human, amplified cDNA was hybridized to the Human Genome U133 Plus 2.0 gene chips.

Microarray quality control and preprocessing were performed using BioConductor in R. Preprocessing of the three experiments, 1) spontaneous AA mice vs. normal mice, 2) prevention mice with three treatments vs. placebo and sham-operated mice, and 3) treatment mice for two treatments vs. placebo were performed separately using the same pipeline.

Quality control was performed using the affyanalysisQC package from http://arrayanalysis.org/. AffyanalysisQC uses the R/BioConductor packages: affy, affycomp, affypdnn, affyPLM, affyQCReport, ArrayTools, bioDistm biomaRt, simpleaffy, and yaqcaffy to perform QC within a single script. RMA normalization was performed on each experimental group separately. Batch effect correction using ComBat was required for the prevention experiments. Batches, treatments and time points were modeled treating each treatment group effect as constant over time, and grouping the PBS controls in groups reflecting both treatment and time.

In addition to the preprocessing that was done for the mouse skin samples, Harshlight was used to correct for image defects for the human skin samples.

Data Deposition

Microarray and RNA-seq data was deposited in Gene Expression Omnibus, accession numbers GSE45657, GSE45512, GSE45513, GSE45514, GSE45551, and GSE58573.

Identification of Gene Signatures

Differential Expression Analysis

Initial analysis of differential gene expression was performed on the spontaneous mouse 3×3 and the human 5×5 data sets using limma. A threshold of 1.5 fold change and unadjusted p-value of 0.05.

Unsupervised Analysis

Hierarchical clustering was performed using Cluster on the 363 genes from the human 5×5 and 583 genes from the spontaneous mouse 3×3 that met the threshold abs(log FC) >1, unadjusted p-value <=0.05. Genes were median centered and normalized. Spearman rank correlation was used as the similarity measure and average linkage was used to perform row (genes) and column (sample) clustering. Visualization of the hierarchical clusters was performed with java TreeView. Gene Expression Dynamic Index (GEDI) analysis was used to visualize how “metagenes” identified with a self organizing map algorithm vary across samples. Metagenes are clusters of genes that show similar expression patterns across samples and that are assigned to a single pixel in a two dimensional grid. Neighboring pixels demonstrate similar expression patterns to one another.

RT-PCR Validation

Predicted differentially expressed genes in human and mouse were confirmed using RT-PCR. First-strand cDNA was synthesized using a ratio of 2:1 random primers: Oligo (dT) primer and SuperScript III RT (Invitrogen) according to the manufacturer's instructions. qRT-PCR was performed on an ABI 7300 machine and analyzed with ABI Relative Quantification Study software (Applied Biosystems, Foster City, Calif., USA). Primers were designed according to ABI guidelines and all reactions were performed using Power SYBR Green PCR Master Mix (Applied Biosystems), 250 nM primers (Invitrogen) and 20 ng cDNA in a 20 μL reaction volume. The following PCR protocol was used: step 1: 50° C. for 2 min; step 2: 95° C. for 10 min; step 3: 95° C. for 15 s; step 4: 60° C. for 1 min; repeat steps 3 and 4 for 40 cycles. All samples were run in quadruplicate for three independent runs and normalized against an endogenous internal control as indicated.

ALADIN Scores

The IFN and CTL signatures were used to develop a bivariate score statistic. Individual signature IFN and CTL scores were determined following procedures used in human SLE. The sets of genes selected to comprise the IFN and CTL signatures were CD8A, GZMB, and ICOS for the CTL signature, and CXCL9, CXCL10, CXCL11, STAT1, and MX1 for the IFN signature. The scores for the prevention mice were calculated in relation to the sham mice; whereas, the scores for the topical treatment experiments were calculated relative to all the samples at week zero. Based on the human studies, ALADIN was further extended to include a hair keratin (KER) signature. The set of genes selected to comprise the KER signature are DSG4, HOXC31, KRT31, KRT32, KT33B, KRT82, PKP1, and PKP2. The ALADIN scores for the baseline and 12 week skin biopsies obtained from subjects enrolled in the oral Ruxolitinib clinical trial were calculated relative to the healthy controls at baseline.

Power Analysis

For the analysis of response to treatment, the inventors performed a two-sample comparison of proportions power calculation for group sample sizes of five each for treated and placebo mice for the case when the true proportion in population 1 (the treatment group) expected to respond to treatment is 0.95 and the true proportion in population 2 (the placebo group) expected to respond is 0.20. At a significance level of alpha=0.05, using Barnard's exact test the inventors calculated a power of 0.803 for a one-sided test to detect a difference of proportions when the proportions for the two populations are 0.95 and 0.20 with group sample sizes equal to five each. In some cases in which fewer than 5 animals per group were present per experiment, multiple experiments were collapsed in order to ensure statistical power.

Statistical Analysis of Treatment Effects

Mice were expected to exhibit alopecia 4-12 weeks after grafting of alopecic skin. Experiments in which control mice failed to demonstrate hair loss by 8 weeks were aborted. For the prevention experiments, a time-to-event survival analysis for interval censored data was performed. The survival and interval packages in R were used to perform log-rank tests. Hair growth index was calculated.

For the treatment experiments (FIG. 14B), the R package nparLD was used to test the hypothesis that there exists a treatment by time interaction. Analyses were performed using the hair growth index from three replicate experiments containing three mice from each treatment and placebo group for a total of nine mice from each group. A F1-LD-F1 design was employed. For the JAK1/2i treatment vs. placebo, the hypothesis of no interaction, i.e., parallel time profiles, is rejected at the 5% level using both the Wald-Type Statistic and the ANOVA-Type Statistic with the p-values of 4.40e-21 and 3.35e-18, respectively. For the JAK3i Treatment vs Placebo, the hypothesis of no interaction, i.e., parallel time profiles, is rejected at the 5% level using both the Wald-Type Statistic and the ANOVA-Type Statistic with the p-values of 1.45e-30 and 2.42e-21, respectively.

All mice were included in survival (time-to-event) analysis statistics. For lymph node and skin cell analysis, biopsy was harvested at the indicated time points following treatment in parallel with control mice. In the IFN-γ- and IL-2-neutralization experiments one out of five control mice that did not exhibit hair loss was not included in the photographs. These mice were not sacrificed in order to continue to monitor for hair loss, but for statistical purposes for skin cell analysis, these unanalyzed samples were assigned a cell count value of 0% CD8+NKG2D+ cells to allow for a rigorous and conservative statistical comparison with treated mice.

No randomization was used and the investigators were not blinded to the group allocation during the experiments or when assessing the outcomes.

Unpaired parametric two-sided t-tests were used to test for differences in means and frequencies between treated and untreated groups. For statistical purposes, the inventors assume all variances to be the same for each group.

Interval censored log-rank tests were used to perform all time to event survival analysis. This test properly accounts for data where the exact event time is not known but the event is known to fall within some interval.

Nonparametric longitudinal data analysis was used to test for response x time interactions. These methods are particularly suited for small sample size.

Sample Sizes, Number of Replicates, and Statistical Tests Among Experiments

| FIG. | n_control | n_exp | experiment | statistic | p-value |
|---|---|---|---|---|---|
| 1c | 3 + 3 + 3 + 3 | 6 + 6 + 6 + 6 | C3HAA/C3H cell counts | t | <0.0001 |
| 1d | 2 + 2 + 2 | 5 + 5 + 4 | C3HAA/C3H skin | t | <0.0001 |
| 1d | 3 + 3 + 2 | 4 + 4 + 4 | C3HAA/C3H lymph node | t | <0.0001 |
| 1f | 3 | 3 | primary cell culture | N/A | NA |
| 2b | 5 | 5 | α-IFNg, mice with hair loss | log-rank | 0.047 |
| 2b | 5 | 5 | α-IFNg Skin cell counts | t | 0.0228 |
| 2e | 5 | 5 | α-IL2, mice wth hair loss | log-rank | 0.048 |
| 2e | 5 | 5 | α-IL2 Skin cell counts | t | 0.0091 |
| 2h | 5 + 4 + 3 | 5 + 4 + 3 | α-IL15Rb, mice with hair loss | log-rank | 1.11E−05 |
| 2h | 2 + 2 + 1 | 2 + 2 + 2 | α-IL15Rh Skin cell counts | t | <0.0001 |
| 3b | 4 + 6 | 4 + 6 | JAK1/2i, mice with hair loss | log-rank | 0.00041 |
| 3c | 2 + 3 | 3 + 3 | JAK1/2i Skin cell counts | t | 0.0003 |
| 3c | 2 + 3 | 4 + 5 | JAK1/2i lymph node cell counts | t | <0.0001 |
| 3g | 7 | 5 | JAK3i, mice with hair kiss | log-rank | 0.0025 |
| 3h | 2 + 2 | 2 + 3 | JAK3i Skin cell counts | t | 0.0002 |
| 3h | 2 + 2 | 2 + 3 | JAK3i lymph node cell counts | t | 0.0049 |
| 4b | 3 + 3 + 3 | 3 + 3 + 3 | JAK1/2i/JAK3i/Vehicle | nonparLD | Jak1/2i 4.4e−21, |
| | | | | | Jak3i 1.5e−30 |
| 4c | 3 | 3 | JAK1/2i/JAK3i/Vehicle Skin | t | Jak1/2i 0.017, |
| | | | | | Jak3 0.015 |
| 4c | 3 | 3 | JAK1/2i/JAK3i/Vehicle lymph | t | Jak1/2i p = 0.0297, |
| | | | node | | Jak3i p = 0.0908 |

For in vivo studies data are provided as cumulative data. The number of replicates are provided as shown above; For example “3+3+3+3” refers to four separate experiments each including three experimental mice. For in vitro studies, experiments were performed in triplicate.

### 6.3 Example 3

Summary

Alopecia areata (AA) is a highly prevalent autoimmune disease in the United States with a lifetime risk of 1.7%. However, AA remains a significant unmet need in dermatology, and treatments are lacking. Janus kinase inhibitors are emerging as potential therapies for many autoimmune conditions, including most recently AA. The inventors report a patient who was treated with oral tofacitinib citrate, a preferential JAK3/JAK1 inhibitor, for AA, resulting in significant hair regrowth and concurrent skin and blood biomarker changes. The inventors hypothesized that effective tofacitinib treatment of alopecia areata would be accompanied by changes in expression of AA-associated genes in skin as well as circulating serum CXCL10 levels. Punch biopsies were taken at baseline and after four weeks of treatment. Total RNA was extracted, reverse-transcribed, ampli-feed, biotinylated and then hybridized to Human U133 Plus 2.0 gene chips (Affymetrix, Santa Clara, Calif.). ALADIN scores were calculated as previously described relative to healthy controls at baseline. Serum from blood draws taken prior to the initiation of tofaci-tinib treatment and after four weeks of treatment were assayed for CXCL10 levels using the Human IP-10/CXCL10 ELISA kit (Sigma-Aldrich, St. Louis, Mo.) according to the manufacturer's instructions.

Results

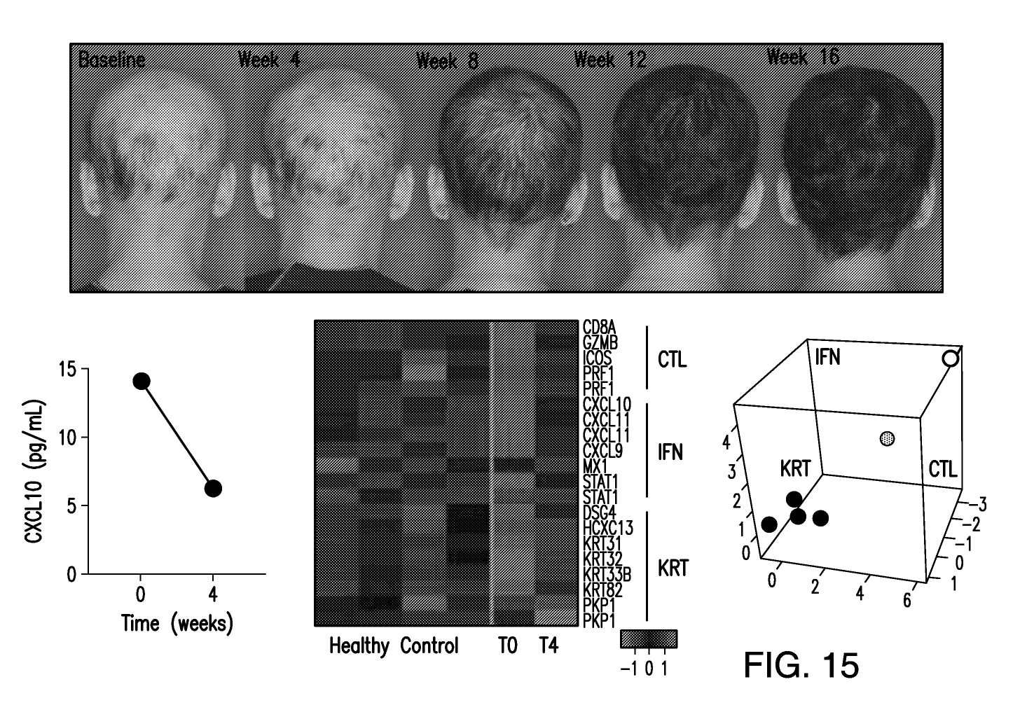

A 40-year-old Caucasian woman with persistent moderate/severe AA was enrolled in the open-label pilot study to test the efficacy of oral tofacitinib for AA (https://clinicaltrials.gov/NCT02299297). Her AA began on her scalp 5 years prior to enrolment and resolved completely within 1 year in the setting of pregnancy. A few months after delivery, her AA recurred as patchy disease. Treatment with topical corticosteroids, anthralin cream and intra-lesional corticosteroids was of limited benefit. Her AA progressed to involve all extremities, eyelashes and eyebrows with patchy scalp involvement and remained stable until her enrollment into the clinical trial (FIG. 15). Her past medical history was unremarkable, and she denied a family history of AA.

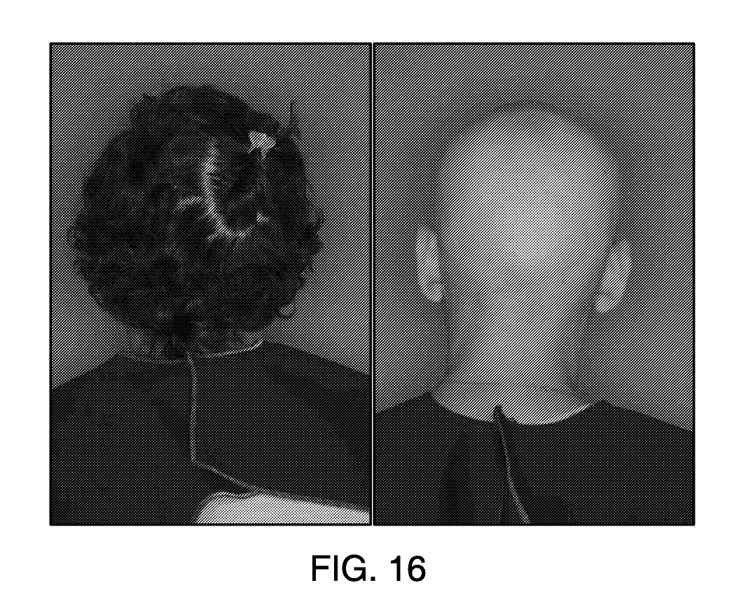

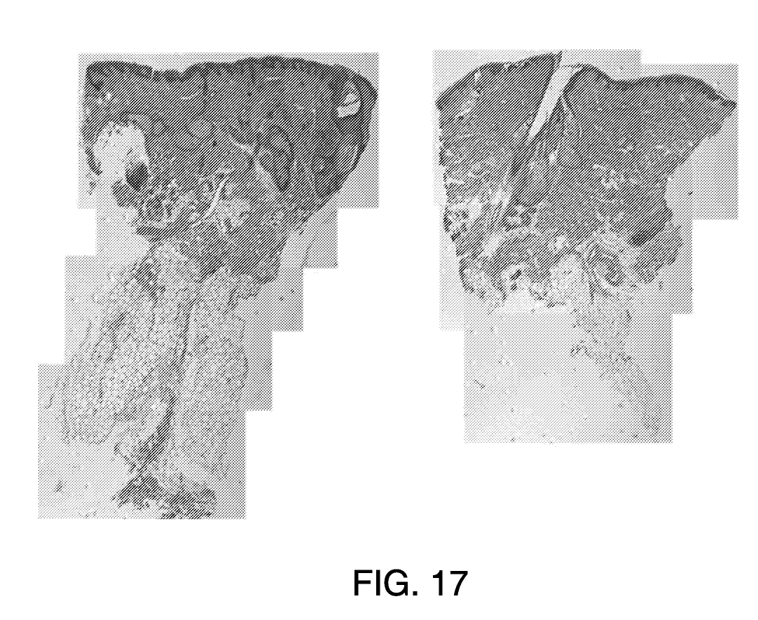

The patient began treatment with tofacitinib 5 mg twice daily. Patchy regrowth was noted at month 1. After two and three months of treatment, she had scalp hair regrowth of 62.5% and 94%, respectively. Significant regrowth of her eyebrows and eyelashes was noted. Scalp hair regrowth was nearly complete 4 months after initiating treatment (FIG. 15). There were no adverse events reported and no laboratory abnormalities in her complete blood count, complete metabolic panel or lipid profile. Cessation of treatment with tofacitinib resulted in near-complete hair loss (FIG. 16).

Punch biopsies of the scalp (FIG. 17) and blood draws were performed at baseline and after 4 weeks of treatment to monitor gene expression and biomarker changes. Serum levels of CXCL10, an interferon (IFN)-induced chemokine found at high levels in AA skin, decreased after 4 weeks of treatment (FIG. 17). In addition, microarray analysis was performed on the skin biopsy samples. Based on the AA Disease Activity Index (ALADIN), the patient exhibited high IFN and cytotoxic T lymphocyte (CTL) signatures at baseline that decreased by 4 weeks of treatment, although not to the level of normal controls (FIG. 17).

Conclusion

Alopecia areata is an autoimmune disease with strong associations with genetic loci in close proximity to genes with immune functions. Targeting candidate immune pathways that may be con-tributing to disease pathogenesis is an active area of investigation, and JAK inhibitors target multiple immune signalling path-ways involved in AA. The inventors have previously shown systemic and topical tofacitinib to be effective in preventing the development of AA, as well as reversing established AA, in the graft model of AA in C3H/HeJ mice. The inventors report here effective treatment of a human subject with persistent patchy AA, correlating with a diminished ALADIN profile compared to baseline.

### 6.4 Example 4

Summary

Alopecia areata (AA) is a common autoimmune disease with a lifetime risk of 1.7%, for which there are no FDA-approved treatments. The inventors previously identified a dominant IFNg transcriptional signature in cytotoxic T cells (CTLs) in human and mouse AA skin, and showed that treatment with JAK inhibitors induced durable hair regrowth in mice by targeting this pathway. Here, the inventors investigated the use of the oral JAK1/2 inhibitor ruxolitinib in the treatment of patients with moderate to severe AA.

The inventors initiated an open-label clinical trial of 12 patients with moderate to severe AA, using oral ruxolitinib 20 mg BID for 3-6 months of treatment followed by 3 months follow-up off drug. The primary end-point was the proportion of subjects with 50% or greater hair regrowth from baseline to end of treatment.

Nine of twelve patients (75%) demonstrated a remarkable response to treatment, with average hair regrowth of 92% at the end of treatment. Safety parameters remained largely within normal limits and no serious adverse effects were reported. Gene expression profiling revealed treatment related downregulation of inflammatory markers, including signatures for CTLs and IFN response genes and upregulation of hair specific markers.

In this pilot study, 9 of 12 patients (75%) treated with ruxolitinib showed significant scalp hair regrowth and improvement of AA. Larger, randomized, controlled trials are needed to further assess the safety and efficacy of ruxolitinib in the treatment of AA.

Introduction

Alopecia areata (AA) is a major medical problem and is among the most prevalent autoimmune diseases in the US, with a lifetime risk of 1.7%. AA affects both genders across all ethnicities, and represents the second most common form of human hair loss, second only to androgenetic alopecia. AA usually presents with patchy hair loss. One-third of these patients will experience spontaneous remissions within the first year. However, many patients' disease will progress to alopecia totalis (AT, total scalp hair loss) or alopecia universalis (AU, loss of all body hair). Persistent moderate/severe AA causes significant disfigurement and psychological distress to affected individuals. In clinical practice, there are no evidence-based treatments for AA, yet various treatments are offered, most commonly topical and intralesional steroids which have limited efficacy.

Recent studies demonstrated a dominant role for type I cellular immunity in AA pathogenesis, mediated by interferon-gamma producing NKG2D-bearing CD8+ cytotoxic T lymphocytes (CTLs). The central role of type I cellular immunity is also reflected in the transcriptional landscape of AA lesional skin in humans and mice, which is dominated by IFN response genes and a CTL signature. These findings provided the rationale for therapeutically targeting JAK1/2 kinases in AA, and indeed the inventors showed that treatment with JAK inhibitors reversed AA in C3H/HeJ mice, and eliminated the Type I inflammatory response in the skin.

On the basis of the preclinical findings, the inventors initiated a Phase 2 efficacy signal-seeking clinical trial in moderate to severe AA, assessing the clinical and immunopathological response to treatment with oral ruxolitinib, a JAK1/2 inhibitor currently FDA-approved for the treatment of myeloproliferative disorders.

Methods

Study Design, Oversight, and Participants

The study was conceived and conducted by the investigative team at Columbia University. All authors had access to the data and attest to its accuracy and for the fidelity of this report to the study protocol. This study was conducted in accordance with Good Clinical Practice (GCP), as defined by the International Conference on Harmonization (ICH) and in accordance with the ethical principles underlying European Union Directive 2001/20/EC and the United States Code of Federal Regulations, Title 21, Part 50 (21CFR50). Prior to study initiation, approval was obtained from the Columbia University IRB for the protocol and all study related materials. Freely given written informed consent was obtained from every subject before screening or study-related procedures. Monitoring for regulatory compliance and adherence to the IRB approved protocol was performed by the Columbia University Clinical Trials Office and the Department of Surgery Regulatory Team. The study was registered on clinicaltrials.gov prior to initiation. The inventors enrolled 12 adult patients, including 10 patients with moderate to severe AA (30-95% hair loss) and 2 patients with AT or AU.

Study Assessments and Outcomes

The study's primary efficacy endpoint was the proportion of responders at end of treatment, defined as those subjects achieving at least 50% regrowth compared to baseline assessed by the Severity of Alopecia Tool (SALT) score, a standardized, validated method for estimating hair loss in AA. Secondary efficacy endpoints included hair re-growth as a continuous variable. Additionally, Quality of Life measures (Dermatology Quality of Life Index—DLQI and Skindex) were done at regular pre-specified intervals, but did not show statistical differences in comparisons performed (data not shown). To assess response durability, responders were followed for 3 months after treatment was completed. Safety analysis was included as a secondary endpoint for all subjects who received at least one dose of ruxolitinib and was monitored as described at monthly visits.

Exclusion Criteria

Patients were excluded if they had AA for less than 3 months; active, unstable or regrowing AA; were on concomitant treatment (within 1 month prior to enrollment) which could affect hair regrowth; or had evidence of underlying infections, malignancies, immunocompromise or unstable medical conditions. Also excluded were patients with concomitant skin disease on the scalp; or patients taking experimental medications within the last month or three half-lives of the medication. Patients reporting recent or DMARDs (disease modifying anti rheumatologic drugs) use were excluded.

Adverse Effects

Adverse events were categorized as any new untoward medical occurrence (sign, symptom or abnormal laboratory finding) or worsening of a pre-existing medical condition in a patient who took at least one dose of study medication, whether or not the event was considered to have a causal relationship with study treatment. Adverse events were assessed at every monthly visit. Patients were also encouraged to contact the study center in the interim between visits if they developed new signs or symptoms of concern. Several patients developed modest declines of white blood cell counts initially but levels remained within normal limits and therefore no dose adjustment was required. One patient developed lowered hemoglobin levels, which required dose modification. No significant decline in platelet counts were observed. One patient developed 2 episodes of reported furuncles/abscesses. Both episodes were evaluated by the patient's primary doctor and had resolved before the patient was evaluated by the research team. The same patient also reported a possible biopsy site infection for which she sought medical attention and was reportedly treated with oral antibiotics while out of the country. Several patients developed mild URIs deemed to be seasonal and unrelated to medication. One patient had a mild episode of pneumonia, confirmed via chest x-ray, which was treated with oral antibiotics. This patient had a distant history of an episode of pneumonia years prior to study participation. There were no observed clinically significant occurrences of lowered platelets. No hepatic abnormalities were observed. One patient had elevated lipids at baseline and during treatment. He was monitored by his primary physician while on study drug, and had no clinically apparent adverse effects related to lipid levels. Two patients developed lesions consistent with acne or scalp folliculitis. Both episodes resolved within weeks. Three patients had GI symptoms including diarrhea. One patient developed conjunctival hemorrhage following a pre-planned ophthalmic procedure (a commonly seen side effect of the procedure) and transient hemorrhoid bleeding. There was no concomitant change in circulating levels of platelets. Several patients had minor abnormalities on urine analysis/microscopy. One patient was treated for urinary tract infection.

Biomarker Assessment and Clinical Correlative Studies

Biopsies and peripheral blood were obtained at baseline and after 12 weeks for immune monitoring and molecular studies. Several patients provided additional biopsies at intermediate time points during the course of treatment, and one patient provided an additional sample at week 24. Tissues specimens were fixed and stored in PAXgene Tissue Containers. Total RNA was extracted from skin biopsy specimens harvested during the course of the clinical trial using the PAXgene tissue miRNA kit. Library prep was performed for microarray analysis using Ovation RNA Amplification System V2 and Biotin Encore kits (NuGen Technologies, Inc., San Carlos, Calif.). Samples were subsequently hybridized to Human Genome U133 Plus 2.0 chips (Affymetrix, Santa Clara, Calif.) and scanned at the Yale Center for Genome Analysis. Library prep and microarray hybridization of RNA extracted from skin biopsies from three healthy controls were performed together with the samples from the treated patients for a total of 31 samples. Gene expression analyses included calculation of ALADIN scores, differential expression analysis of the expression levels for the identification of gene expression signatures, principal component analysis, and statistical analysis of the ALADIN scores. Microarray data from the 31 samples have been deposited in GEO under accession number GSE80342.

Microarray Preprocessing and Quality Control

Microarray quality control and preprocessing were performed using BioConductor in R. Quality control was performed using the R standalone version of affyAnalysisQC from http://arrayanalysis.org.

Samples which included 31 from this study plus three additional samples from healthy controls from GEO accession number GSE53573 were normalized together using GCRMA. Affymetrix probe sets were called present or absent by affyAnalysisQC using a MASS algorithm implemented in R. Probeset IDs (PSIDs) that were on the X or Y chromosome, that were Affymetrix control probe sets, or that did not have Gene Symbol annotation were removed from all arrays for further downstream analysis. Data were analyzed using log 2(Intensity) for expression levels.

Batch effect correction was required in order to include the three healthy control samples from the earlier dataset GSE58573 for the calculation of ALADIN scores and for increased power in the differential expression analysis. A modified version of ComBat (M-ComBat) was used in order to integrate the normal samples from the earlier GEO dataset. Expression levels of the samples from the current data set were held fixed while M-ComBat was used to integrate the three healthy control samples from GSE58573 with the current healthy control samples. M-ComBat is an implementation of the function ComBat available in the sva package that allows one of the batches to be used as a reference batch.

Calculation of ALADIN Score

ALADIN scores were calculated for all 34 samples using the batch corrected expression data. The CTL, IFN, and KRT ALADIN scores were determined following procedures outlined previously. Briefly, z-scores are calculated for each PSID relative to the mean and standard deviation of normal controls. Scores for each gene are obtained by averaging z-scores of PSIDs mapping to that gene. Signature scores are then calculated averages of the scores for genes belonging to the corresponding signature.

Identification of Gene Expression Signatures

In order to perform further analysis on the ruxolitinib samples at baseline (n=12) with the Normal controls (n=6), PSIDs were further filtered to retain only features that were called present on at least one of the 18 samples, there were 36147 PSIDs remaining for further downstream analysis.

Filtering of Paired Samples at Times t=0 and t=12 from Patients Who Responded to Ruxolitinib Treatment

After QC, there were 8 patients with microarray data at both t=0 and at t=12 who responded to ruxolitinib treatment. PSIDs were further filtered to retain only features that were called present on at least one of the 16 samples, there were 35563 PSIDs remaining for further differential expression analysis.

Differential Expression Analysis

Differential expression analysis was performed on the samples from t=0 and Normal controls and on the paired data from t=0 and t=12 from the responders using linear models as implemented in the limma package in Bioconductor. A threshold of 2.0 fold change and unadjusted p-value of 0.05 was used.

Comparisons were made between responders at baseline and normal controls, between responders and non-responders at baseline, between non-responders at baseline and normal controls, between responders at baseline and at 12 weeks of treatment, and finally between responders at 12 weeks of treatment and normal controls.

Principal Component Analysis of Six Healthy Controls and Ruxolitinib Treated Patients at t=0 and t=12

Principal component analysis was performed on the samples from the six healthy controls and ruxolitinib treated patients at baseline and 12 weeks using the responder signature made up of PSIDs that were differentially expressed between responders at baseline and normal controls.

Statistical Analysis

All variables were examined for distributional assumptions, checked for accuracy and for out of range values. Based on a priori definition the inventors classified patients as a responder if they experienced 50% or greater hair re-growth from baseline, based on the SALT score at end of treatment. The inventors examined the overall distribution of demographic factors, and looked at possible differences between responders and non-responders, tested for significance using Fisher's Exact Test (two sided) for categorical variables, and Mann-Whitney U test for continuous variables. The inventors then examined the change between baseline, end of treatment (EOT), and end of study (EOS) scores on relevant variables overall, and for responders and non-responders employing either a Mann-Whitney U test or Wilcoxon Signed Rank test. To estimate the extent of regrowth across time, the inventors considered both a Generalized Estimating Equation and a Mixed Model approach to model the repeated measures data, and opted for the latter given the strong normality assumption of GEE's and the relatively small sample size. For these mixed models, the inventors first modeled regrowth from baseline to end of treatment where time (in weeks) was the independent variable and then, to assess maintenance of the observed effect, modeled regrowth from end of treatment to end of study where again time was the independent variable. In both models the inventors specified compound symmetry as the initial covariance structure.

Statistical Analysis of ALADIN Score

In order to examine possible differences between each of the CTL, IFN, and KRT ALADIN scores in responders and non-responders, in responders and normal controls and in non-responders and normal controls, the inventors tested for differences among the three groups using a Kruskal-Wallis test, followed by Wilcoxon Rank Sum tests as implemented in the coin package in R. The inventors then examined the change between baseline and ALADIN scores at 12 weeks for responders using Wilcoxon Signed Rank test as implemented in the coin package in R. The inventors further tested for differences in the three scores between responders at 12 weeks and normal controls.

ALADIN scores are defined such that mean CTL, IFN and KRT scores are equal to zero, resulting in mean overall (all patients) scores, responder-only scores, and non-responder-only scores, corresponding to the mean differences between these and the normal controls. Statistically significant differences were observed between overall scores at baseline vs normal controls in CTL (p<0.0002), IFN (p<0.005), and KRT (p<0.0002) scores, between responders-only at baseline vs normal controls in CTL (p<0.0004), IFN (p<0.0004) and KRT (p<0.0004); between overall scores at week 12 vs normal controls in CTL (p<0.04) and IFN (p<0.0004); and between responders-only vs normal controls in KRT (p<0.0007) at alpha=0.05. No statistically significant difference was observed in IFN scores at baseline overall vs normal controls; or in CTL and IFN scores in responders-only at week 12 vs normal controls. No statistically significant differences were observed between non-responders at baseline and normal controls in any of the three ALADIN scores.

Changes in ALADIN scores within individual patients were assessed between baseline and week 12. Statistically significant differences were observed between baseline and week 12 overall in the CTL and IFN scores, with KRT scores reaching marginal significance (alpha=0.05). CTL scores declined from 8.30 to 1.51 (p<0.004), IFN scores declined from 31.08 to −0.37 (p<0.004), and KRT scores increased from −39.36 to −15.02 (p=0.054). Among responders only, CTL scores declined from 9.37 to 1.6 (p<0.008), IFN scores declined from 38.37 to 0.24 (p<0.008), and KRT scores increased from −37.84 to −15.42 (p=0.039). Statistically significant differences were observed between responders and non-responders in CTL and IFN scores at baseline (mean score difference=5.91 and 40.11 p<0.036 and 0.036, respectively), but not in KRT scores (mean score difference=−19.74, p=0.22).

Results

Efficacy

This study was an open-label, clinical trial to investigate ruxolitinib (Jakafi, Incyte Pharmaceuticals) 20 mg PO twice daily in the treatment of moderate/severe AA. All patients received ruxolitinib for 3 to 6 months, followed by a 3 month observational phase to assess treatment response durability.

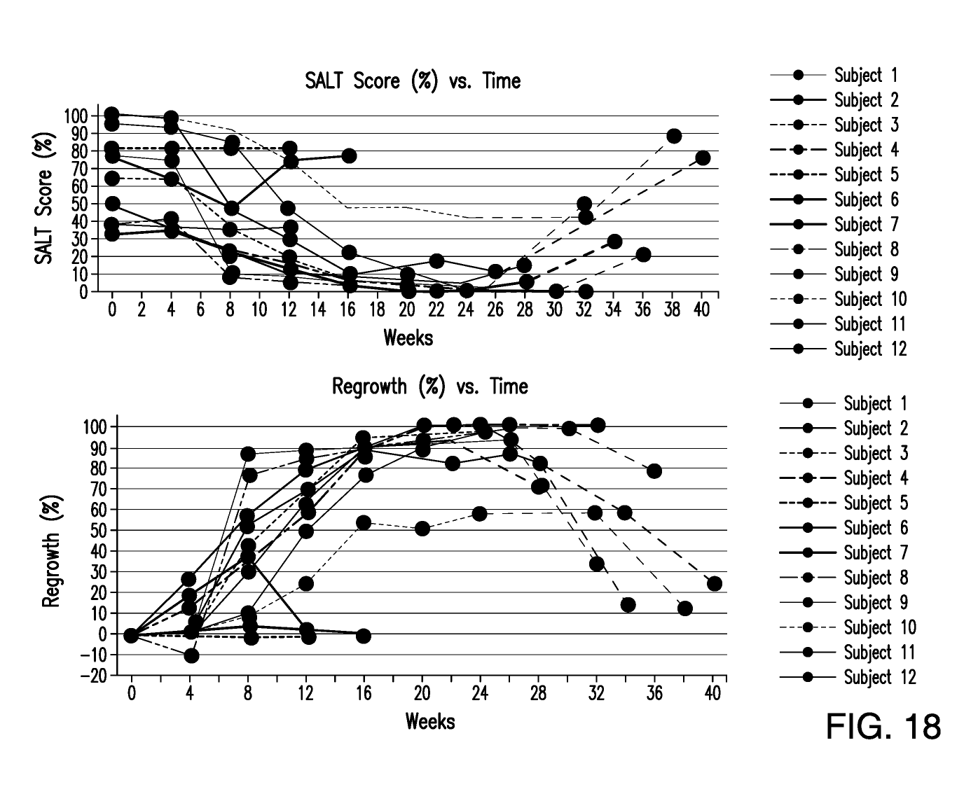

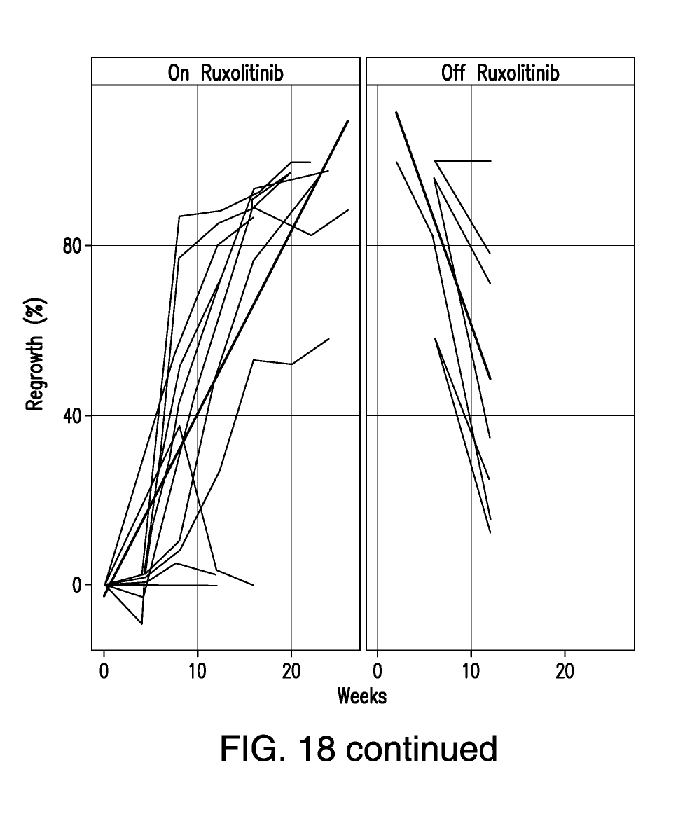

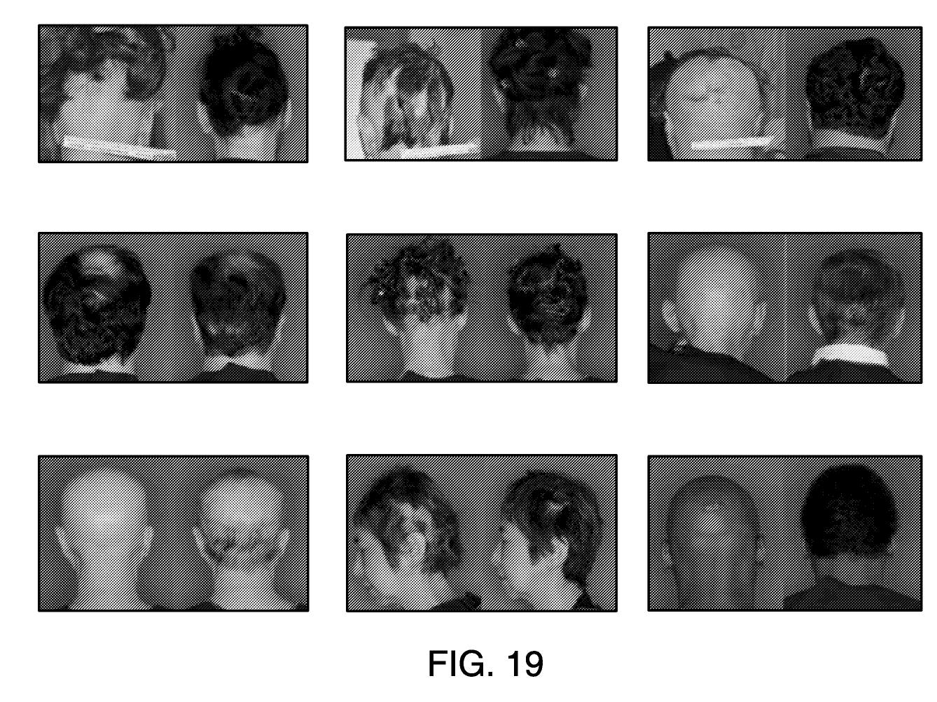

Nine of twelve patients (75%) had significant hair regrowth and achieved the primary outcome of at least 50% regrowth. The mean baseline SALT score of 65.8±28.0% decreased to a score of 24.8±22.9% at 3 months and 7.3±13.5% at the end of 6 months of treatment (p<0.004). As a group, the responders exhibited a 92% reduction in hair loss from baseline (FIGS. 18 and 19), with seven of the nine responders achieving over 95% regrowth by end of treatment.

Regrowth was seen in responders as soon as four weeks after study medication was initiated and initially presented as variably subtle patchy areas of regrowth consisting of pigmented terminal hairs, with the exception of one patient (subject 4) with concurrent vitiligo, who exhibited primarily grey hair regrowth. Of note, the areas of vitiligo in this patient were also noted to improve with ruxolitinib treatment8. Hair regrowth for all responders increased steadily with significant increases each month, resulting in the majority (8 of 9) of responders achieving at least 50% regrowth by the week 12 visit. Responding patients with evidence of regrowth at 3 months continued treatment until the subject had either achieved 95-100% regrowth or completed 6 months of treatment.

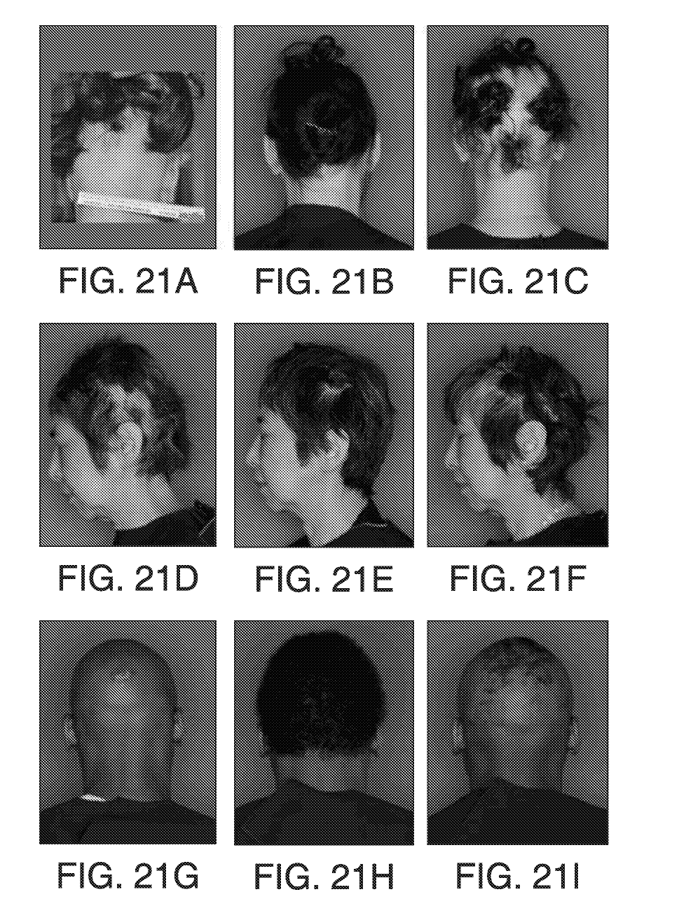

Durability of responses were assessed in the 3 month follow-up period off treatment. Three of nine responders noted shedding beginning at week 3 following ruxolitinib discontinuation and had significant hair loss at week 12 off drug (FIG. 21); however, hair loss did not reach baseline levels (FIG. 18). Six of nine responders reported mild increased shedding.

Biomarker and Clinical Correlative Studies

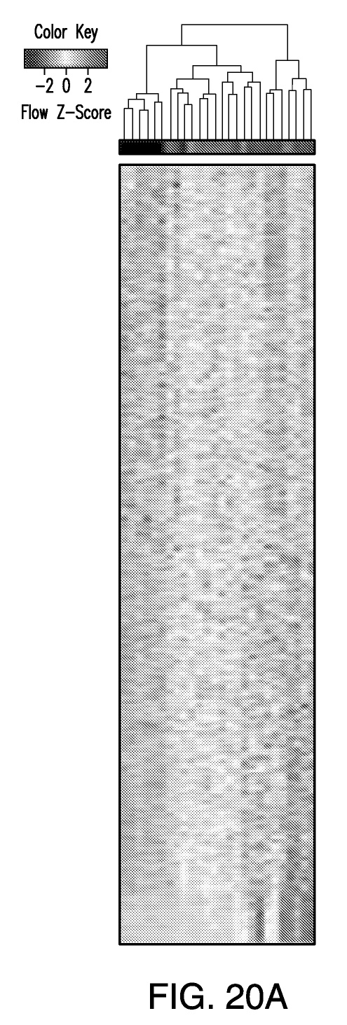

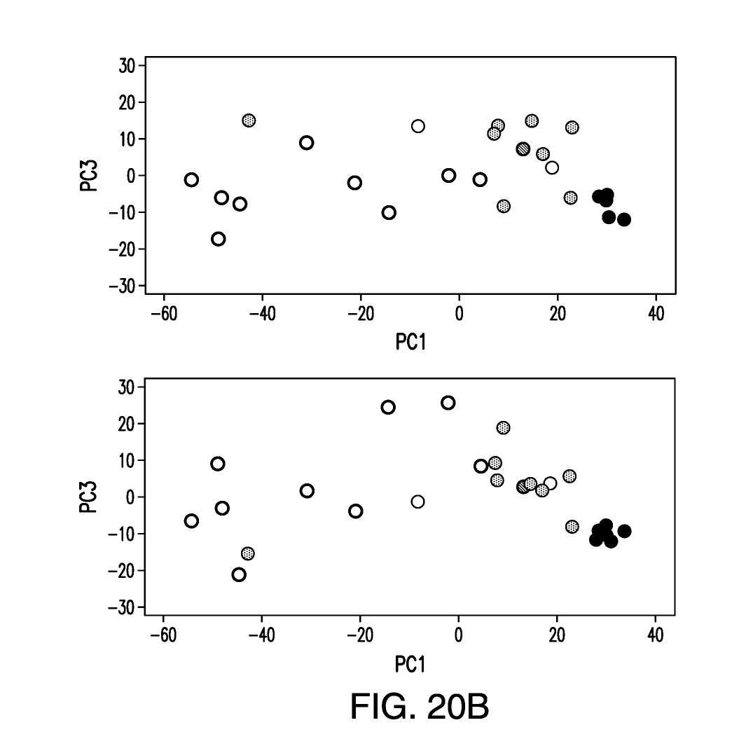

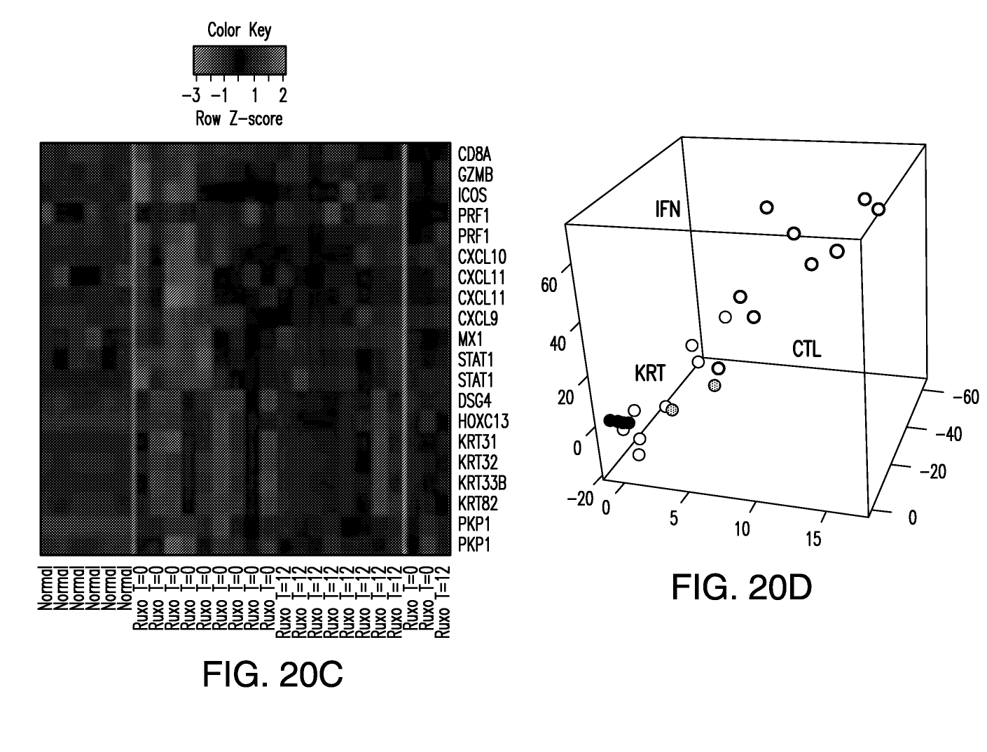

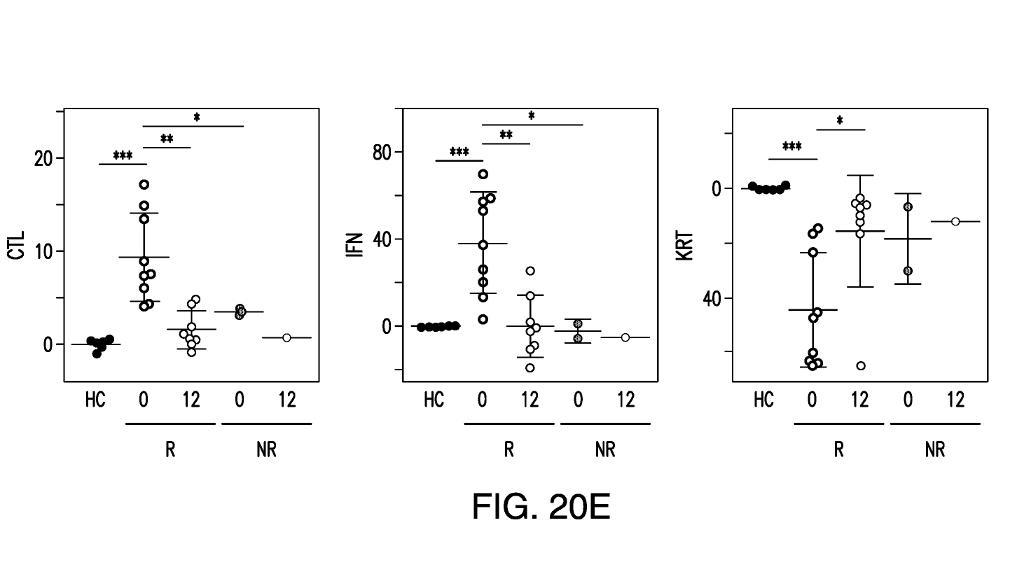

Gene expression profiling was performed on skin biopsies taken at baseline and following twelve weeks of treatment, with additional optional biopsies performed earlier in the treatment course. Baseline scalp samples exhibited a distinct gene expression profile when compared to samples taken from unaffected patients (FIG. 20A). Following ruxolitinib treatment, AA patient scalp samples clustered more closely with healthy control scalp samples than with baseline AA samples (FIG. 20B), indicating global normalization of the AA pathogenic response. Gene expression profiles attributed to the interferon (IFN), cytotoxic T lymphocyte (CTL), and hair keratin (KRT) signatures were assessed in the tri-variate Alopecia Areata Disease Activity Index (ALADIN, FIG. 20C), a summary index of the AA pathogenic inflammatory response and hair regrowth. Importantly, eventual AA responders clustered together on the ALADIN matrix at baseline, sharing high IFN and CTL scores (FIG. 20C, D).

Notably, baseline samples from eventual AA nonresponders exhibited relatively low IFN and CTL scores (FIG. 20D, E) that were not statistically different than normal control samples. Furthermore, in the cohort of AA patients on ruxolitinib treatment as described, the CTL and IFN signature scores were capable of distinguishing eventual nonresponders and responders at baseline (p<0.036 and p<0.036 for CTL and IFN scores, respectively).

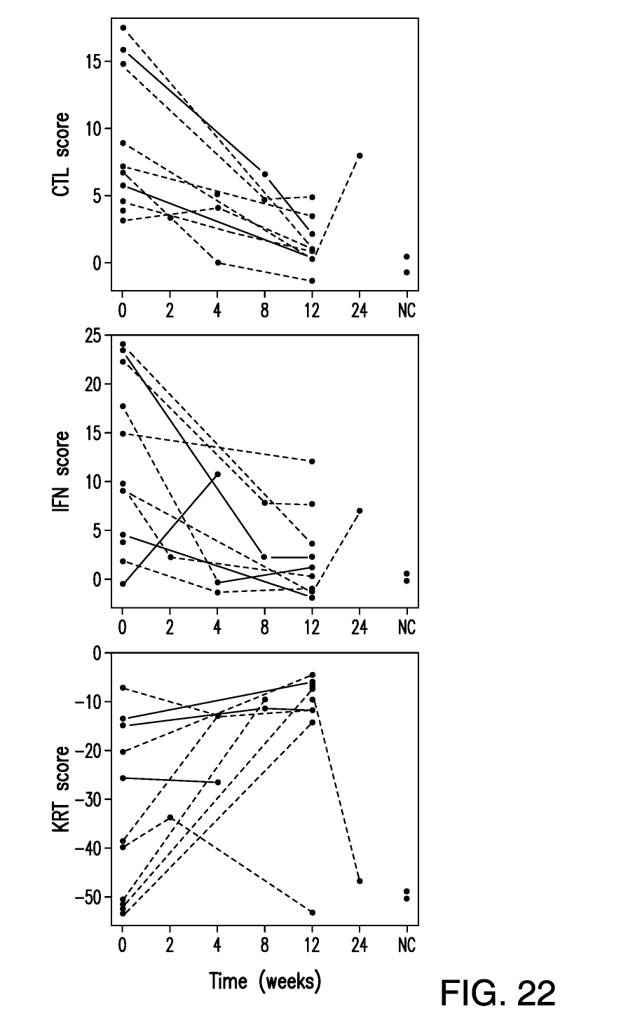

Consistent with on-target activity of treatment, skin samples taken following 12 weeks of treatment from responding patients exhibited much lower IFN and CTL scores and clustered much more closely to, skin samples taken from normal control patients on the ALADIN matrix (FIG. 20D, E) Decreased IFN and CTL scores in post-treatment biopsies were demonstrable as early as 2 weeks after the initiation of treatment (FIG. 22).

Adverse Events

Ruxolitinib was well tolerated and safely administered in all 12 patients. There were no serious adverse effects and no patients required discontinuation of therapy. Observed adverse effects were infrequent and included three minor bacterial skin infections (in the same patient), 9 episodes of URI/allergy symptoms in 7 patients, one UTI, one mild pneumonia, mild GI symptoms, and one conjunctival hemorrhage following a surgical procedure. One patient developed lowered hemoglobin, which resolved with dose modification.

Discussion

In this proof-of-concept study, ruxolitinib 20 mg twice per day for three to six months induced significant hair regrowth in nine of twelve patients, an overall 75% rate of response to ruxolitinib in the treatment of AA. In contrast, the expected spontaneous remission rates (occurrence of hair regrowth, without treatment) in patients with moderate to severe AA is less than 12% based on two randomized controlled trials with similar subject populations. Even the most severe forms of alopecia, AT/AU, responded indicating that the autoimmune process remains pathogenically active and remains reversible with JAK inhibition. Hair regrowth was evident within one month in responders and progressed at a rapid rate. Responses were near complete by 6 months of treatment in 8 of 9 responders, suggesting that 6 months of therapy is sufficient to induce maximal clinical remissions in the majority of responders.

In this 9 month study, ruxolitinib was well tolerated. The safety signals in this small study of AA patients, who are otherwise healthy, compare favorably with the prior clinical experience for ruxolitinib in patients with myeloproliferative disorders, in which adverse events, particularly hematologically-related, are understandably more frequent, and are consistent with findings from use of tofacitinib in the treatment of patients with psoriasis.

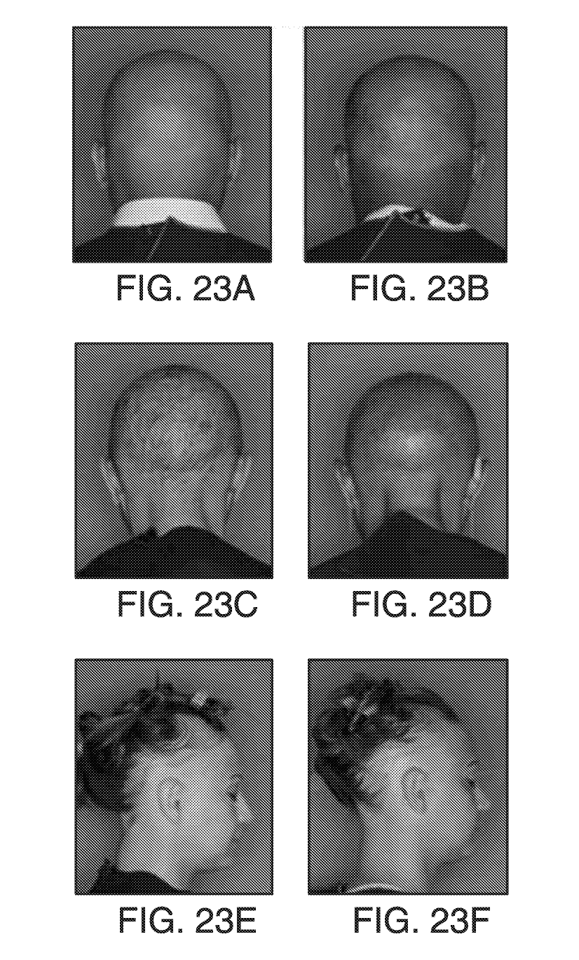

Transcriptional profiling of paired baseline and on-treatment scalp biopsies was both mechanistically and clinically informative. Baseline skin samples from responders had high inflammatory ALADIN IFN and CTL scores with near normalization after 12 weeks of treatment, indicative of JAK1/2i mediated suppression of the autoreactive CD8 T cell response. Indeed, early ALADIN normalization, as early as week 2 following initiating treatment (FIG. 22), may be predictive of favorable week twelve clinical outcomes. Conversely, nonresponder samples exhibited low baseline IFN/CTL scores and clustered relatively closely to normal patient samples, suggestive of alternative inflammatory or non-inflammatory etiologies of hair loss in these non-responders (FIG. 23). One nonresponder had both AA and androgenic alopecia, another nonresponder's alopecia was consistent with AA histologically but appeared to be a rare diffuse form of the disease, and the final nonresponder exhibited an ophiasis AA pattern.

Recent single case reports have described clinical responses in AA patients treated with other JAK inhibitors including tofacitinib, ruxolitinib, and baricitinib. These proof-of-concept data demonstrate immunopathological reversibility of the Type I inflammatory response that underlies AA, even in patients with long-standing or the more severe forms of disease, providing a strong rationale for clinical development of oral and/or topical JAK inhibitors for the treatment of AA.

### 6.5 Example 5

Summary

Network-based molecular modeling of physiological behaviors has proven invaluable in the study of com-plex diseases such as cancer, but these approaches remain largely untested in contexts involving interact-ing tissues such as in autoimmunity. Here, using Alopecia Areata (AA) as a model, the inventors have adapted regulatory network analysis to specifically isolate physiological behaviors in the skin that contribute to the recruitment of immune cells in autoimmune dis-ease. The inventors use context-specific regulatory networks to deconvolve and identify skin-specific regulatory modules with IKZF1 and DLX4 as master regulators (MRs). These MRs are sufficient to induce AA-like mo-lecular states in vitro in three cultured cell lines, re-sulting in induced NKG2D-dependent cytotoxicity. This work demonstrates the feasibility of a network-based approach for compartmentalizing and target-ing molecular behaviors contributing to interactions between tissues in autoimmune disease.

Introduction

Systems-level analysis using reverse-engineered regulatory networks is an emerging computational discipline that has demonstrated great promise in the study of complex diseases such as cancer and Alzheimer's disease. This approach enables the modeling of complex physiological behaviors as modules of genes (subsets of differentially expressed genes that associate with disease) that are controlled by master regulators (MRs). MRs represent the minimal number of transcription factors (TFs) that are predicted to specifically activate or repress a target module and, by extension, the associated physiological behavior. They can be regarded as molecular “switches” that regulate physiological behaviors. The inference of MRs is made possible through the reverse engineering of context-specific regulatory networks using computational algorithms such as ARACNe.

These MRs are validated biologically and serve as targetable “hubs” governing disease pathology. These approaches have proven highly effective for the study of cell autonomous behaviors in diseases such as cancer. Physiological behaviors such as mesenchymal transformation in glioblastoma and oncogenesis in B cell lymphoma or breast cancer, as well as onset of Alzheimer's disease have been functionally linked to a relatively small number of MRs, which in turn become the “bottleneck” that can be used to infer driver mutations in patients or become the targets of drug screens for treatment.

However, this type of computational approach is only starting to be implemented to target pathogenic, non-cell autonomous interactions between different tissues such as autoimmune disease. In particular, inferring MRs cannot be done directly using typical ARACNe-based analysis because of fundamental assumptions made during the generation of a regulatory network: (1) that the samples used are relatively pure or represent the one underlying transcriptional network; and (2) the underlying molecular behavior of a data set exists at a steady state such that each sample can be treated as a “snapshot” of regulatory dependency within the overall network. A contaminated sample, particularly by a tissue that exhibits a different context-specific regulatory network, can impair the accuracy of regulatory predictions. Further, when pathogenesis is dependent on the interaction between the two tissues, there will always be an artifact correlation between contaminant gene signatures and the molecular modules that recruit them, but are expressed in the other tissue. This makes it difficult to clearly define modules exclusive to one tissue or the other when analyzing gene expression data generated from a mixture of the two tissues.

Alopecia Areata (AA) provides an ideal model for such a study since it is characterized by cytotoxic T cells actively infiltrating the hair follicles and scalp skin that are typically absent in normal skin. AA typically presents as loss of distinct, random patches of hair that can spread to the entire scalp (alopecia totalis) or the entire body (alopecia universalis). Previous research has directly implicated immune genes in AA, many of which are shared with other autoimmune diseases such as type 1 diabetes, celiac disease, and rheumatoid arthritis. Previous studies have identified infiltration of cytotoxic CD8-positive, NKG2D-positive T cells into the skin of AA, and the pathology of AA involves IFN-gamma-dependent signaling pathways, which are frequently disrupted in association with immune evasion in cancer.

Little work has been done to determine if there are intrinsic factors in the “end organ” (the tissue that suffers autoimmune attack) that contribute to the disease, such as scalp skin in AA, making this molecular component a prime target for the analysis. The inventors predict that pathogenic changes in the molecular profile of the scalp skin will contain genes that mediate interactions with the infiltrating T cells. As a corollary, identifying the MRs will grant regulatory control over the modules that are sufficient to induce immune recruitment. To study this, the inventors leverage context-specific regulatory networks for the regulatory deconvolution of a mixed-signature gene expression profile of AA patients. The goal of this work was to develop a framework capable of separating mixed AA tissue biopsy gene expression data into skin-specific modules of AA pathology and infiltrate recruitment.

The inventors identified a molecular profile of AA that includes the genetic modules of infiltrate recruitment in the scalp skin by filtering genes that do not accurately map to a skin-specific network. This scalp skin signature allowed the subsequent identification of two MRs of scalp skin contribution to infiltration: IKZF1 and DLX4. These two genes are expressed in primary scalp biopsies and are sufficient to induce an AA-like molecular signature and NKG2D-dependent cytotoxicity in independent, wild-type cellular contexts, allowing for direct genetic induction of immune-mediated cytotoxicity.

Results

Initial Definition of a Pathogenic Expression Signature in AA Reveals the Presence of Local Scalp Skin and Infiltrating Immune Signals

First, the inventors created a molecular signature comparing AA patients to controls to generate a molecular representation of AA. The inventors analyzed a training set of microarray studies of patient biopsies from an initial cohort of 34 unique biopsy samples: 21 AA patients of varying clinical presentations and 13 unaffected controls. The inventors additionally had patient-matched, nonlesional scalp biopsies for 12 of the 21 AA patients. These 34 patients were gathered as the first of two cohorts totaling 96 patients, the remainder of which was saved for validation studies.

The inventors created an overall gene expression signature by comparing patients of two distinct clinical presentations, patchy AA (AAP) and totalis and universalis (AT/AU) all against unaffected controls. To account for artifacts in the signature associated with secondary effects of infiltration such as hair loss, the inventors then performed hierarchical clustering using this gene signature on a set of patient-matched lesional (symptomatic skin with hair loss) and nonlesional (asymptomatic hair-bearing skin) samples. This analysis identified gene clusters that were differentially expressed between these samples and those that were systemically equivalent across lesional and nonlesional samples. The inventors subsequently removed from the first expression set any genes that fell in clusters correlating with lesional versus nonlesional states. This primarily removed a significant number (but not all) of the keratin and keratin-associated proteins from the signature.

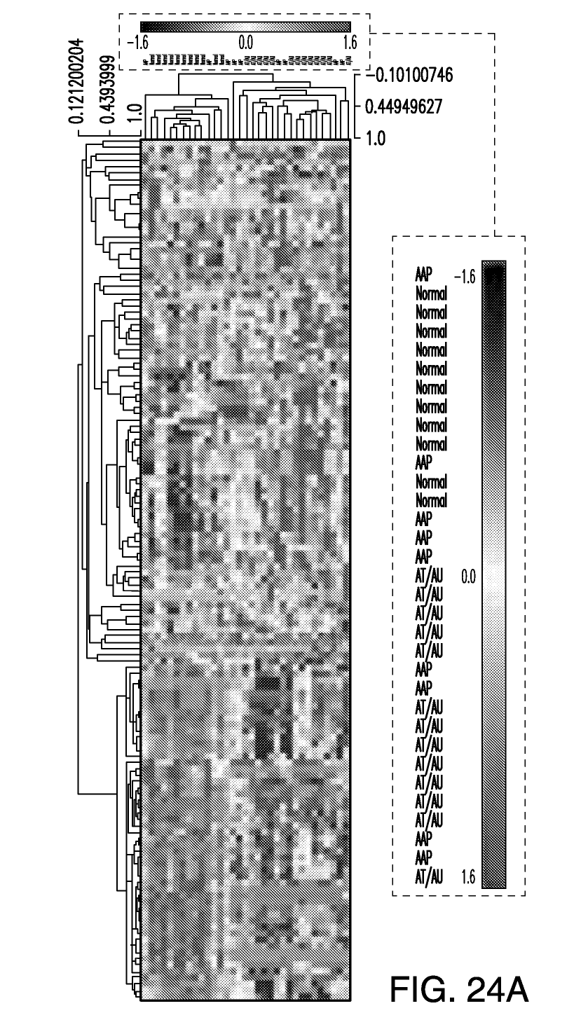

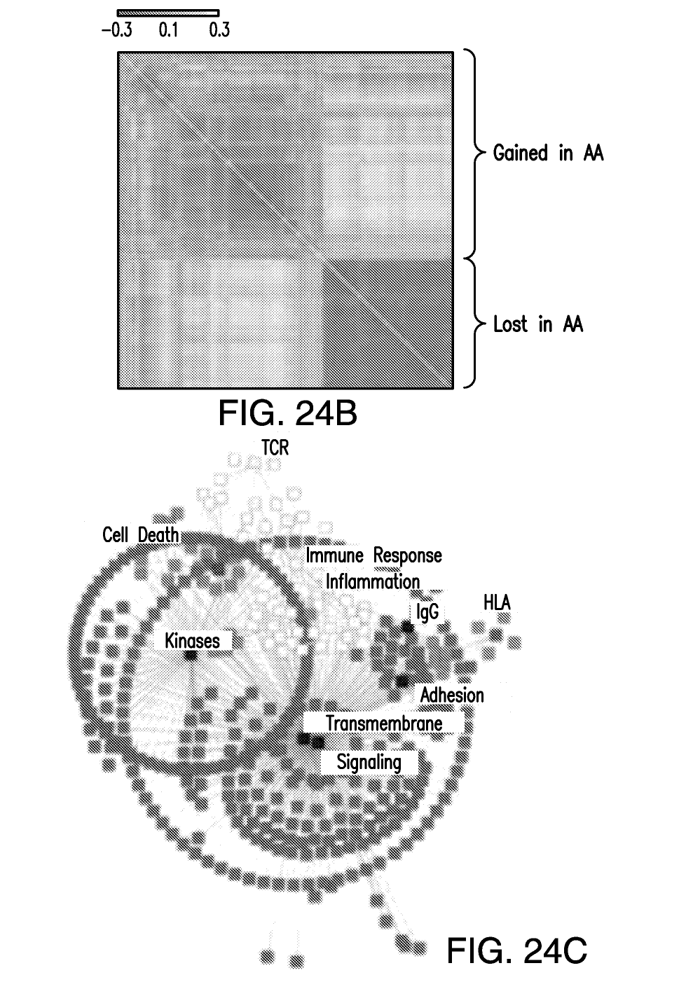

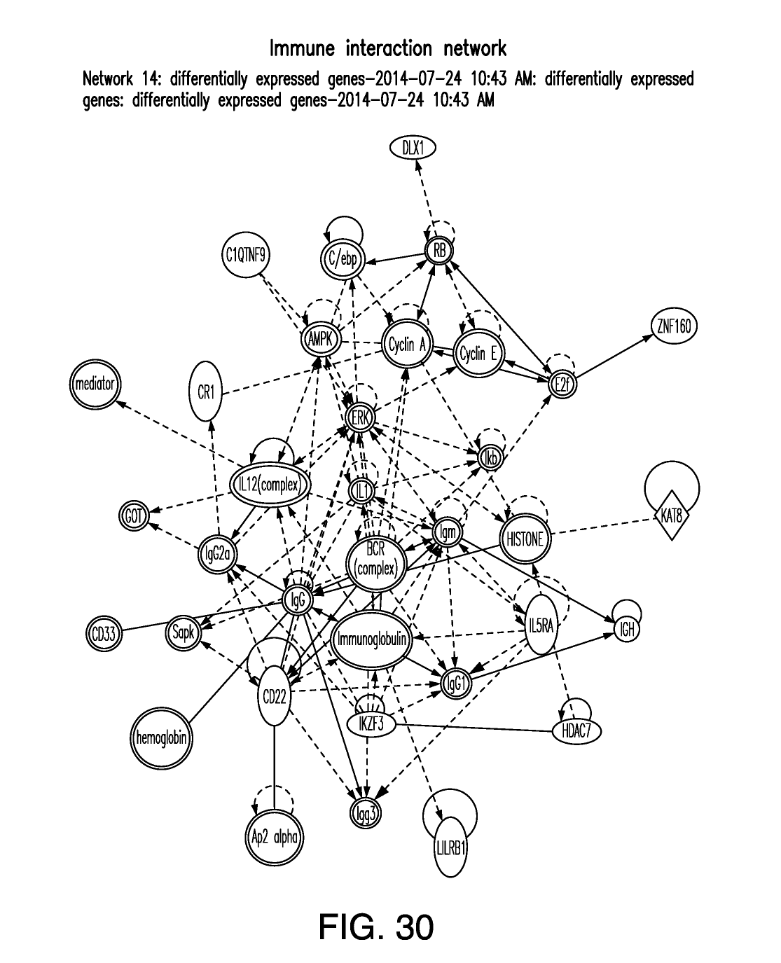

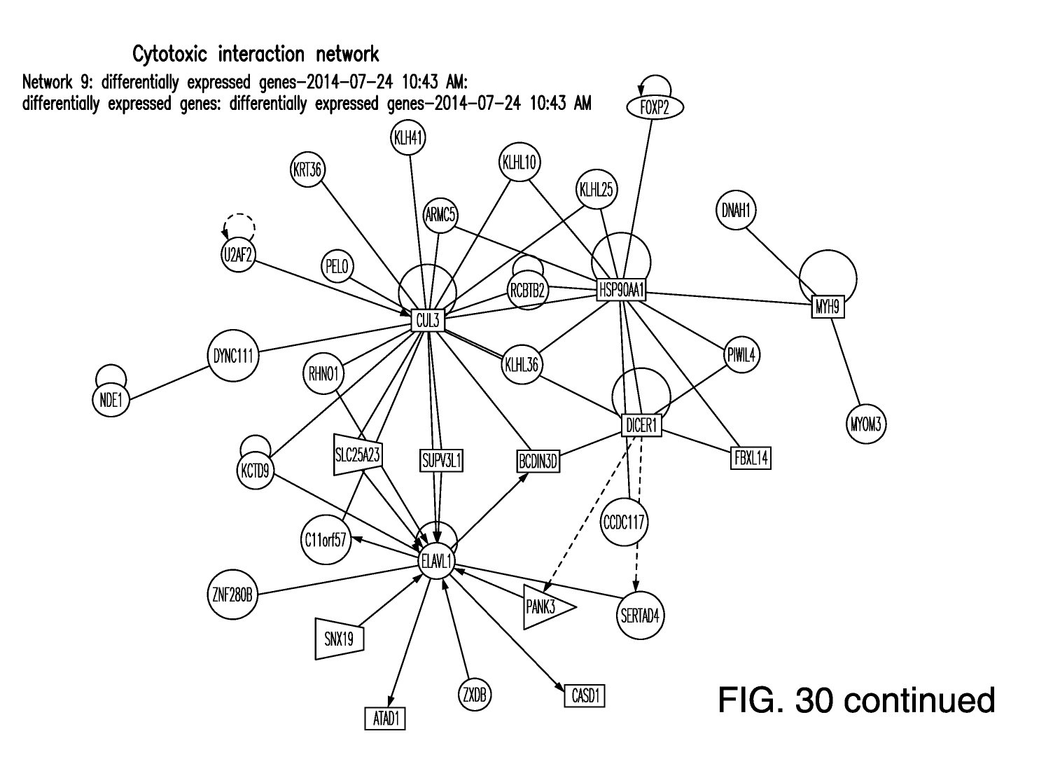

The resulting gene expression signature, the Alopecia Areata Gene Signature (AAGS), consisted of a total of 136 unique genes (Table A) and provided sufficient information to cluster the entire training cohort into two appropriate superclusters corresponding to the control and disease states (FIG. 24A). Clustering these genes by co-expression also revealed two distinct modules of genes, with greater diversity of co-expression in the genes upregulated in the disease state (FIG. 24B). As a qualitative measure of the genes differentially expressed between affected and unaffected patients, the inventors analyzed them for functional annotation enrichments. The analysis revealed the presence of HLA genes, immune response elements, and inflammatory and cell death pathway gene expression in the affected patient samples (FIG. 24C). The two most significant superclusters of the AAGS were transmembrane signaling peptides (p=2.8×10−11) and secreted cell-cell signaling peptides (p=2.1×10−10). As expected, this list also contains several antigen-presenting elements and immune response elements that are associated with AA and autoimmune disease (FIG. 30). These results indicate that there are significant alterations of multiple biological processes in AA-presenting cells. The inventors postulate that some subset of these genes originate from the scalp skin and are required to induce infiltration recruitment.

There is also significant evidence for immune-related genes originating from infiltrating immune cells that must be filtered beforehand; or else they could confound the identification of skin-specific molecular programs. Gene markers associated with immune cells or immune response were detected as part of the AAGS including CD8a, CXCL9/10, and CCL5/18/20/26. In primary patient biopsy samples, defining skin-specific molecular behaviors contributing to AA is a difficult task due to the presence of infiltrating T cells and secondary response pathways in AA skin samples.

Leveraging Regulatory Networks to Deconvolve Skin and Immune Signatures in the AAGS into Regulatory Modules

With clear definitions of the disease signature, the inventors sought to deconvolve the skin molecular program in the AAGS from the molecular program originating in infiltrating immune cells in a systemic, unbiased manner. Rather than using GO pathway enrichment or other annotation-based methods that rely on a priori knowledge and potentially ambiguous annotations, the inventors instead utilize the inferred regulatory networks under the hypothesis that the inventors can filter nonskin (immune infiltrate) gene expression by identifying the genes that cannot be mapped to a skin-specific regulatory network.

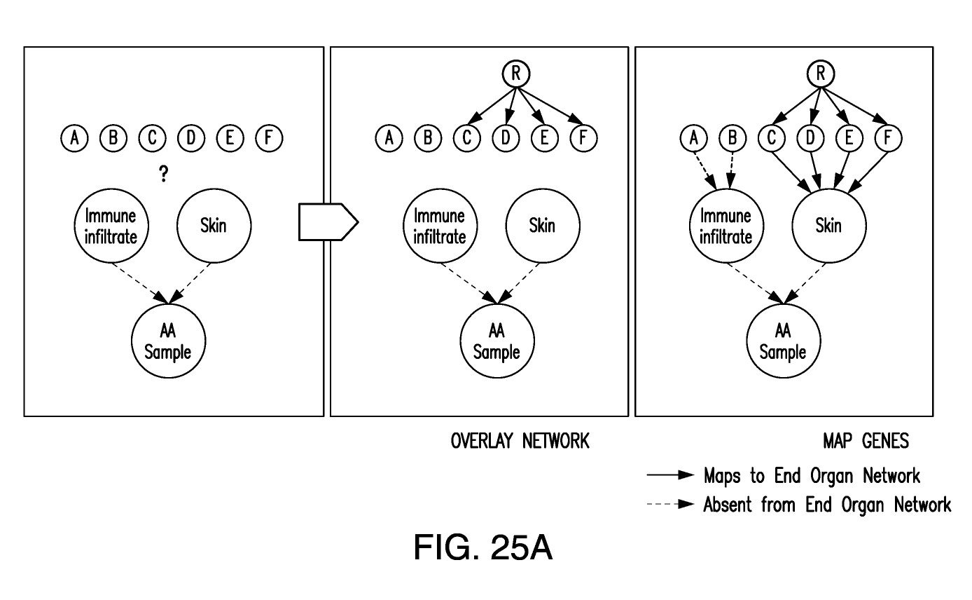

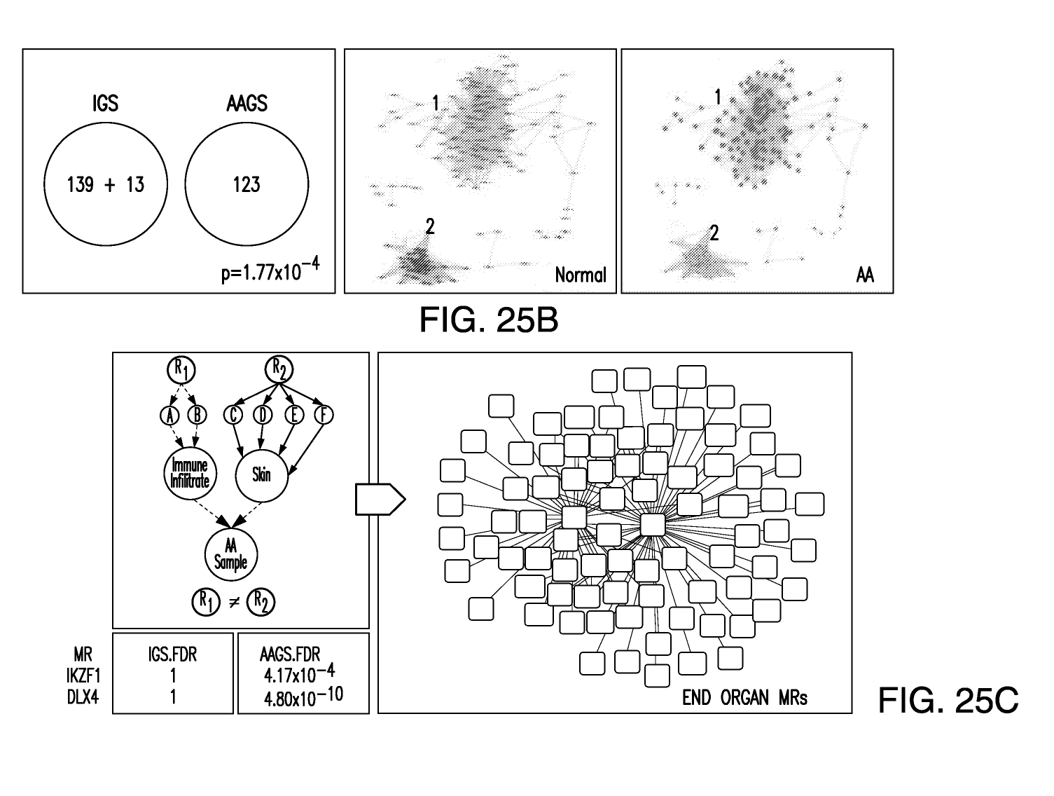

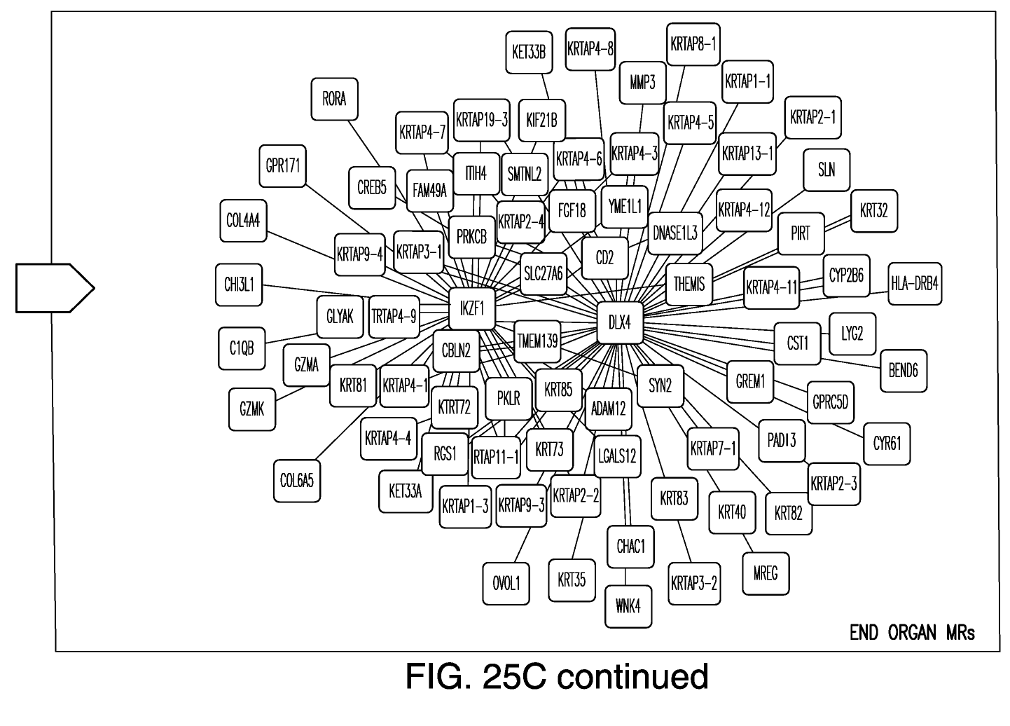

A transcriptional regulatory network of the scalp skin was generated using the ARACNe algorithm and associated software suites (see Experimental Procedures). Specifically, to generate the network, the inventors included a cohort of 106 primary scalp skin samples consisting of normal (unaffected) whole skin biopsies and several samples of primary cultured dermal fibroblasts and dermal papilla cells, which contain few or no T cell infiltrates. This network represents the regulatory network in uninfiltrated skin-derived tissues and serves as the cornerstone of the deconvolution, which occurs in two primary steps as detailed in FIG. 25.

For deconvolution of regulatory modules, the genes in the AAGS are directly mapped to the regulatory network (FIG. 25A; see Experimental Procedures for details). A gene in the AAGS is only retained if there is a direct regulatory interaction between it and a TF using the regulatory logic of a skin ARACNe network (red, solid edges). Any genes that come uniquely from infiltrating immune cells will not have significant representation in the ARACNe network, and are subsequently removed from the AAGS (black, dotted edges) for skin, and added to an Immune Gene Signature (IGS).

The IGS was used as a “negative control” signature, adapted from previous work in characterizing cancer immune infiltrates. The signatures were defined as a set of genes that are specifically expressed in each immune cell type, including T cells, B cells, mast cells, and macrophages. This step iteratively re-defines the AAGS and IGS by separating those genes whose regulation can be accounted for by an uninfiltrated regulatory network (AAGS) from those that cannot (IGS). By extension, the inventors expected the filtered AAGS to be enriched enough in skin gene expression to generate accurate skin-specific regulons.

As indicated in FIG. 25B, 13 infiltrate-specific genes were removed from the AAGS (9.5% of the total signature) when passed through the skin-specific regulatory network. These genes are also listed in Table A. This resulted in two mutually exclusive gene modules (no overlapping genes, p=1.77×10−4), the AAGS and the IGS. A subsequent pathway enrichment analysis further confirmed loss of statistical enrichment of the “T cell activation” and “Immune response” categories, while retaining the other clusters including known skin immune response elements (such as the HLA genes). This left a total of 123 genes in the AAGS that the inventors interpret to represent all end-organ programs associated with AA pathology, including end-organ-initiated immune recruitment and immune response (Table A, starred entries).

Note that the inventors have made the distinction between annotations associated with immune cells (e.g., CD8a) and annotations associated with immune response genes (e.g., HLA). The former are removed by the regulatory network as unrepresented in a skin regulatory network. The latter are signature genes that the inventors aim to keep, as they represent the response elements in the skin and are relevant for the pathology of the disease.

Clustering the filtered AAGS revealed two distinct molecular modules that define the transition from unaffected patients (FIG. 25B, second) to an AA disease state (FIG. 25B, third). Each node represents a gene in the signature, and its size represents the relative expression in each state (larger means higher expression). The inventors labeled these gene groups: (1) genes whose expression is increased when transitioning into the disease state, and (2) genes whose expression is lost in the transition. This filtered AAGS reflects end-organ-specific gene modules and served as the input to the MR analysis.

IKZF1 and DLX4 Are MRs of the Skin AAGS and, by Extension, Infiltrate Recruitment

The next step is the most important in identifying end-organ-specific MRs. The inventors performed MR analyses on both the deconvolved AAGS and the IGS independently and in parallel using the scalp skin regulatory network (FIG. 25C, first, red outline). Using only regulatory interactions represented in skin, the inventors identified the transcriptional regulators that had the highest specificity for the deconvolved AAGS (red arrows) and repeated the analysis for the IGS (black arrows). This step compares the AAGS against the IGS in terms of regulatory logic in the scalp skin, as opposed to direct coverage of gene expression. This analysis assays which TFs are the best candidates for the deconvolved AAGS (and not for the IGS) using a molecular regulatory network specific to the skin. The inventors identify skin-specific candidate MRs by keeping only the candidates that were both significant in AAGS coverage and insignificant for IGS coverage.

Of the significant candidate MRs specifically for the AAGS, the inventors employed a greedy sort to identify the fewest number of regulators needed to maximize the coverage of the AAGS. The inventors found that two MRs were sufficient to cover >60% of the AAGS: IKZF1 and DLX4. Any additional candidates boosted the coverage by a statistically insignificant margin (<5%). The inventors conclude that the maximum AAGS fidelity (most faithful recreation of the expression signature) and efficiency (fewest necessary regulators) could be achieved through these two genes (IKZF1 p=4.17×10⁻⁴ and DLX4 p=4.8×10⁻¹⁰ FDR-corrected).

An equivalent MR analysis conducted on the IGS modules failed to generate any statistically significant or meaningful MRs when using the scalp-skin regulatory network. Specifically, the best candidates for the AAGS, IKZF1 and DLX4, fall to statistical irrelevance (falling from first and second to 159th and 210th, respectively, FDR=1) (FIG. 25C, IGS.FDR). Conducting the MR analysis on the AAGS without deconvolution fails to generate MR candidates at the threshold that is typically expected (both in p value and signature coverage) due to the presence of contaminating genes in the signature which cannot accurately be mapped to a MR, but nonetheless count against enrichment in the analysis.

These two candidates represent the minimum number of regulators required to recreate the AAGS using regulatory interactions derived from a specific tissue context (scalp skin), distinct from any immune-specific regulatory modules that were deconvolved away using this method. IKZF1 and DLX4 therefore represent a genetic regulatory module in the scalp skin that contributes to AA pathogenesis (FIG. 25C, last) and may be sufficient to induce infiltration recruitment in an AA-like manner.

The identification of IKZF1 was unexpected, since it is a well-established T cell differentiation factor, though it is not without precedent that IKZF1 may have a role in cells outside the immune system. However, it is important to note that this analysis does not imply that a MR such as IKZF1 has no role in T cells contributing to AA pathogenesis, but rather, that there is significant evidence that IKZF1 additionally functions in the scalp skin to mediate the interactions between the tissues.

Expression of IKZF1 and DLX4 Induces an AAGS-like Signature in Normal Hair Follicle Dermal Papillae and Human Keratinocytes

To validate the MR predictions with functional studies, the inventors exogenously overexpressed IKZF1 and DLX4 in skin-derived cell lines and cultured cells to test for sufficiency in influencing expression of the AAGS. The inventors cloned DLX4 and two isoforms of IKZF1 for exogenous expression in cultured cells. The active IKZF1 isoform served as the experimental arm of the study, while the isoform that lacks a DNA binding domain was included as a negative control (IKZF1δ). The inventors expressed these genes in cultured primary human hair follicle dermal papillae (huDP) and human keratinocytes (HK). This experimental system allowed us to directly test two distinct, but related, hypotheses: (1) IKZF1 and DLX4 can induce AA-like recruitment of immune cells, and (2) they do so through expression in the skin (not the immune infiltrates).

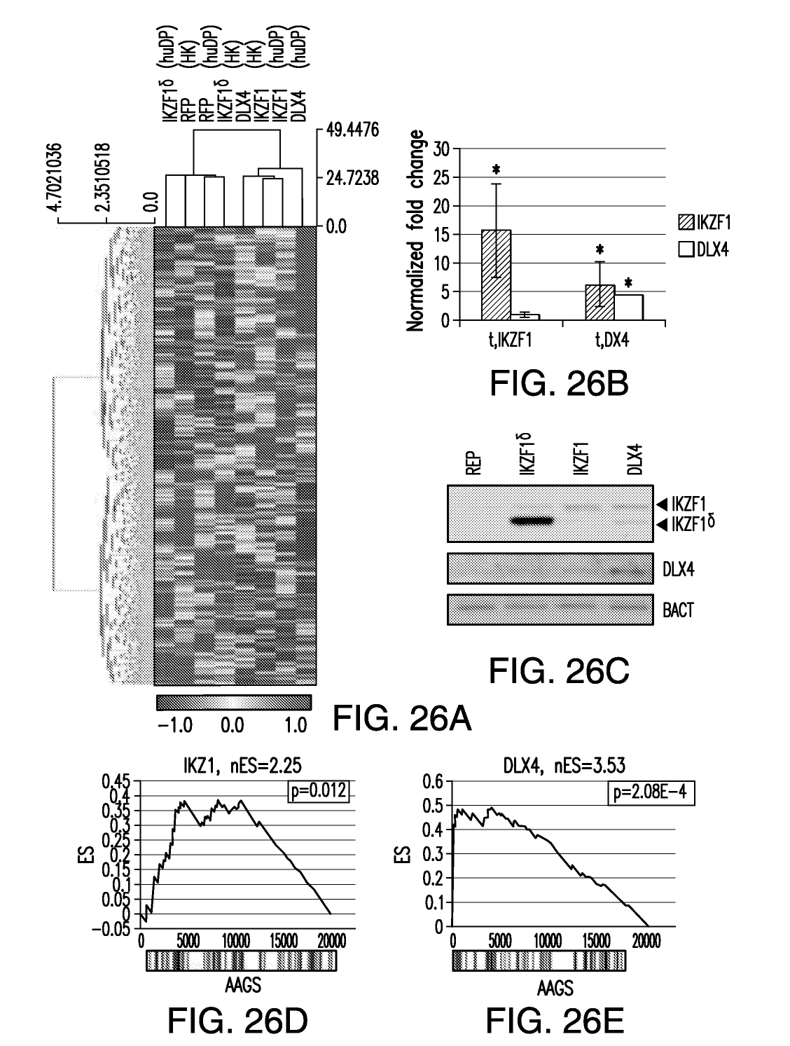

The inventors identified a set of genes that were significantly differentially expressed in the same direction in IKZF1 and DLX4 transfections across both cell types. Unsupervised hierarchical clustering of all samples based on these transcripts reveals clean co-segregation of IKZF1 and DLX4 transfections from IKZF1δ and RFP (red fluorescent protein) controls (FIG. 26A). Furthermore, the inventors observed that the subclustering within these supergroups was not biased based on cell type used (HK did not cluster with HK, and DP did not cluster with DP), supporting that the inventors have identified context-independent effects of MR overexpression. Interestingly, the inventors observed that DLX4 transfections resulted in increased levels of IKZF1 transcript and protein, whereas the IKZF1 transfections did not influence DLX4 expression (FIGS. 26B and 26C).

The inventors subsequently interrogated the expression data for enrichment of the AAGS genes using gene set enrichment analysis (GSEA). The inventors performed two differential gene expression studies comparing the IKZF1 transfections versus RFP controls and DLX4 transfections versus RFP controls. The results show that the ectopic expression of the MRs is followed by significant enrichment in the induction of the AAGS (IKZF1 p=0.012 and DLX4 p=2.08×10⁴; FIGS. 26D and 26E).

IKZF1 and DLX4 Expression are Sufficient to Induce NKG2D-Mediated Cytotoxicity in Normal Cultured Skin

IKZF1 and DLX4 overexpression suggest that these two genes are MRs capable of mediating the AAGS when applied to HK and huDP. However, the functional relevance of these MRs to autoimmunity and immune infiltration is whether or not their expression is sufficient to induce a targeted autoimmune response. In order to investigate this ex vivo, the inventors performed experiments measuring the level of cytotoxic cell death in HK and huDP cells when exposed to peripheral blood mononuclear cells (PBMCs).

The inventors again transfected both HK and huDP cells with one of four expression constructs: IKZF1, DLX4, RFP (negative control), or IKZF1δ (negative control). At 24 hr post-transfection, these cells were incubated with fresh, purified PBMCs. The inventors additionally cultured human dermal fibroblasts and autologous healthy donor PBMCs. The PBMCs were obtained from a healthy control subject with no history of AA or any other autoimmune disease.

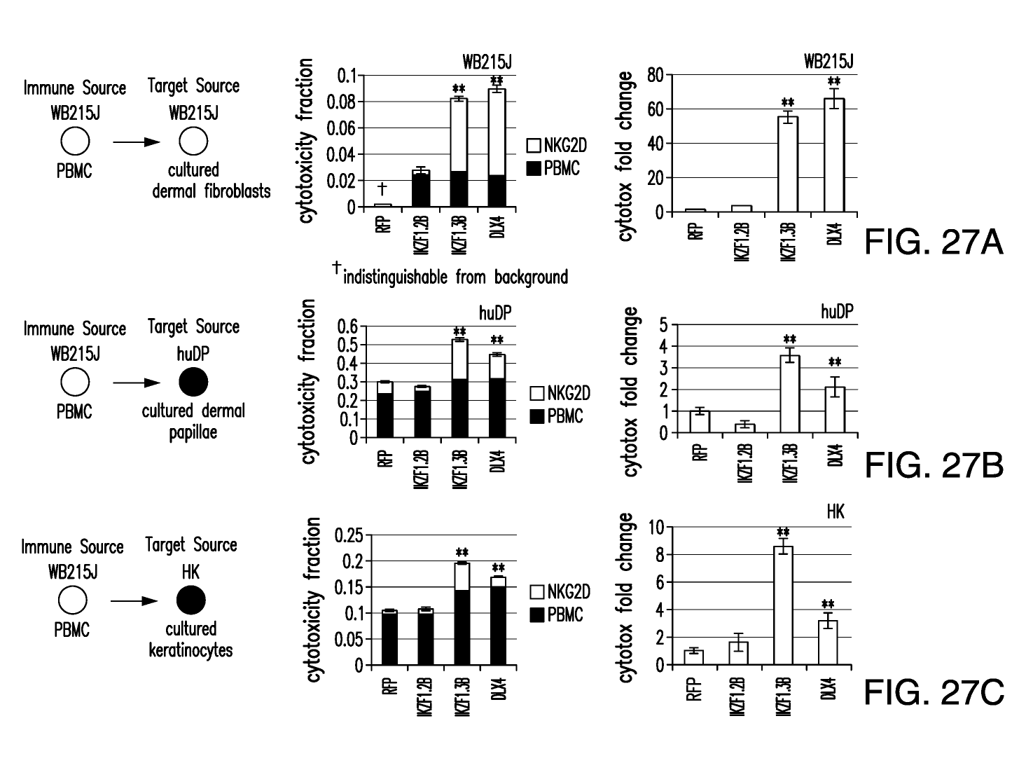

In all comparisons, the inventors observed a statistically significant increase in PBMC-dependent cytotoxicity for the IKZF1 and DLX4 transfections compared to RFP and IKZF1δ controls (FIG. 27, center columns, total bar height). The patient-matched PBMCs and RFP-control transfected fibroblasts exhibited no evidence of cytotoxic interactions, as expected in healthy target cells (FIG. 27A, center). However, the introduction of IKZF1 and DLX4 were both sufficient to induce an interaction between these previously non-interacting cells, resulting in significant increase of total cytotoxicity. In a similar fashion, both huDP (FIG. 27B, center) and HK cells (FIG. 27C, center) showed a significant increase above background levels in cytotoxic sensitivity to the PBMCs.

Since the inventors previously showed that the likely pathogenic immune cells in AA are CD8+NKG2D+ activated T cells, the inventors also performed all treatments with the addition of an NKG2D-blocking antibody (see Experimental Procedures) to prevent NKG2D-dependent interactions. In all cases, the inventors observed that blocking NKG2D suppressed the cytotoxicity in both IKZF1 and DLX4 treatments to levels comparable to controls (FIG. 27, center, gray bars). From the difference between the inhibitor-treated and untreated cells, the inventors can infer the cytotoxicity that is NKG2D-dependent (FIG. 27, center, white bars), which can be normalized to that observed in controls for a relative fold change analysis. From the NKG2D blockade, the inventors observed a statistically significant increase specifically in IKZF1 and DLX4 transfections across all trials (FIG. 27, right). There was a large (>50-fold) increase in patient-matched cytotoxicity compared to the control transfection, which again showed no significant cytotoxicity. There was approximately a 2- to 8-fold increase in NKG2D-dependent cytotoxicity compared to both controls, despite a statistically significant, but small (<10%), increase in NKG2D-independent cytotoxicity. The inventors conclude from these experiments that IKZF1 and DLX4 are capable of inducing NKG2D-dependent interactions with normal PBMCs that result in toxicity for the transfected cells irrespective of the exact tissue type.

Importantly, these experiments establish the cell autonomous function for IKZF1 and DLX4 in the scalp skin as opposed to infiltrating cells, since the exogenous modification was done strictly on normal cultured cells and exposed to healthy PBMCs from a source with no history of autoimmune disease.

MR Expression Permits Reconstruction of a Directional Skin-Specific MR Module of Infiltration Recruitment

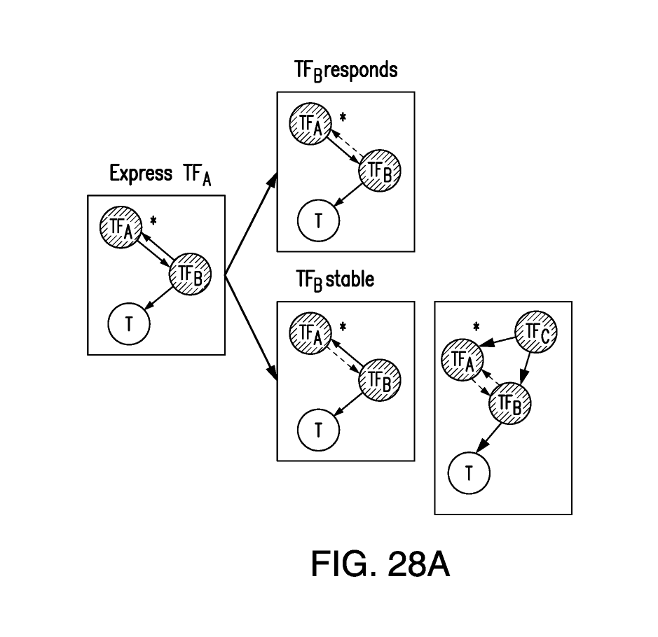

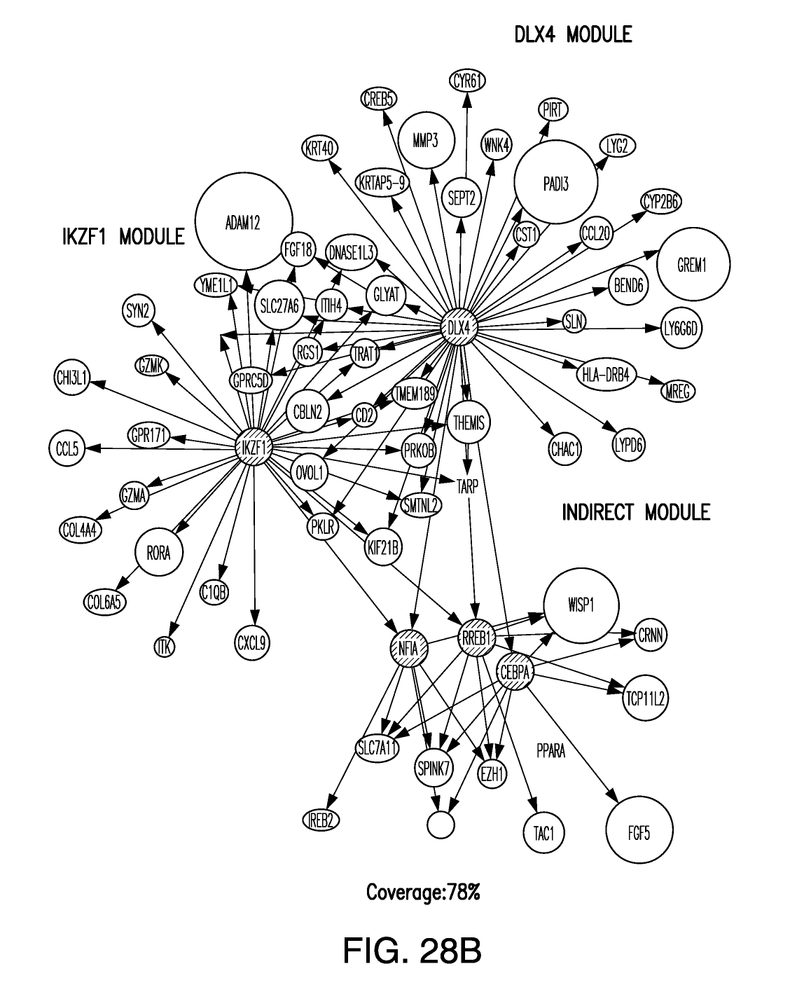

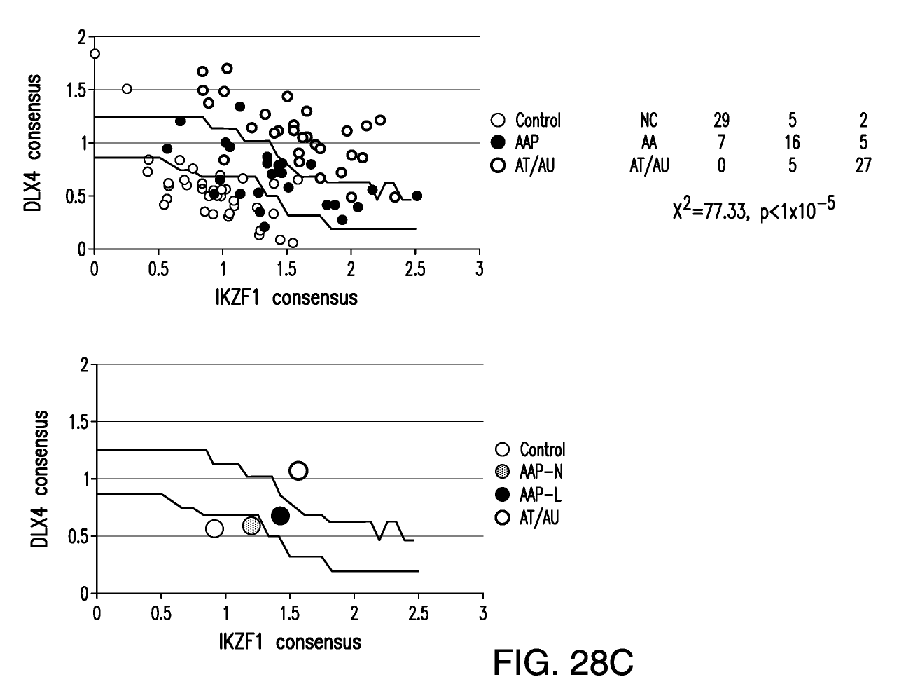

After establishing that IKZF1 and DLX4 are sufficient to induce the AAGS, the inventors sought to use this data to fully reconstruct the AA MR module. ARACNe is capable of detecting direct transcriptional dependencies between a TF and nonregulatory genes that are potential targets (T) because the inventors can infer that the regulation is TF Math Eq T. ARACNe cannot infer directional interactions between TF-TF pairs and subsequently cannot infer secondary T of MRs due to the regulatory equivalence of TFs (FIG. 28A, first). However, since the inventors have directly perturbed HKs and huDPs with specific MRs (FIG. 28A, asterisks), the inventors can use the gene expression data to infer directionality. If TFB is a T of the MR (TFA), then overexpression of TFA will result in the differential expression of TFB and the inventors can infer that TFA Math Eq TFB. Subsequently, any marker genes in the signature associated with TFB can be linked to MR as secondary T TFA Math Eq TFB Math Eq T (FIG. 28A, top). If TFB functions upstream of or in parallel with MR then the expression of TFB and T will not be affected by overexpression of TFA (FIG. 28A, bottom).

Using this logic, the inventors reconstructed the regulatory module to measure the full extent of the coverage obtained by overexpressing IKZF1 and DLX4 in these cellular contexts. The inventors mapped any downstream T of TFs that both (1) respond to IKZF1/DLX4 expression in the experiments, and (2) are predicted to have mutual information with the expressed MR by ARACNe to the regulatory module. The inventors found that 78% of the responding AAGS are within 2° of downstream separation from the MRs IKZF1 and DLX4 based on these criteria (FIG. 28B).

IKZF1 and DLX4 can be Used to Predict Both Immune Infiltration and Disease Severity in an Independent Cohort

As validation of this module, the inventors returned to the original AA array cohort and performed a machine-learning analysis. The inventors attempted to classify a validation AA set into control and affected samples using only the inferred IKZF1 and DLX4 activity. Using the earlier training set from FIG. 24, the inventors arrayed the samples into a search space of two dimensions: the consensus activity of IKZF1 (x axis), and the consensus activity of DLX4 (y axis) (see Experimental Procedures). From the training set, the inventors generated a topographical map of the consensus activity space to define ranges of IZKF1 and DLX4 activity associated with control samples, patchy AA, and AT/AU samples (FIG. 28C, black lines). The region in FIG. 28C closest to the origin of the plot represents the lowest combined IKZF1 and DLX4 activity; its upper bound (the lower black line) is the support vector machine (SVM) margin that maximizes the difference between control and all AA patients. The next upper bound (the upper black line) represents the SVM margin that maximizes the separation of AT/AU patients from AAP.

Using these measures of MR activity, the inventors turned to the validation set and tested for the predictive power of these parameters in separating patients and controls. The inventors observed a strong ability to separate samples into disease and control states, in addition to clinical severity (FIG. 28C, top, p<1×10−5). A centroid map of each patient subgroup more clearly reveals how the transition of patient groups from Control (NC) to AAP and AT/AU is reflected by relative IKZF1 and DLX4 activity (FIG. 28C, bottom). For comparison, the inventors also included a centroid for the AAP nonlesional sample biopsies, which were not included in the training set.

Deconvolution Applied to Independent Inflammatory Skin Diseases Identifies Known Genes

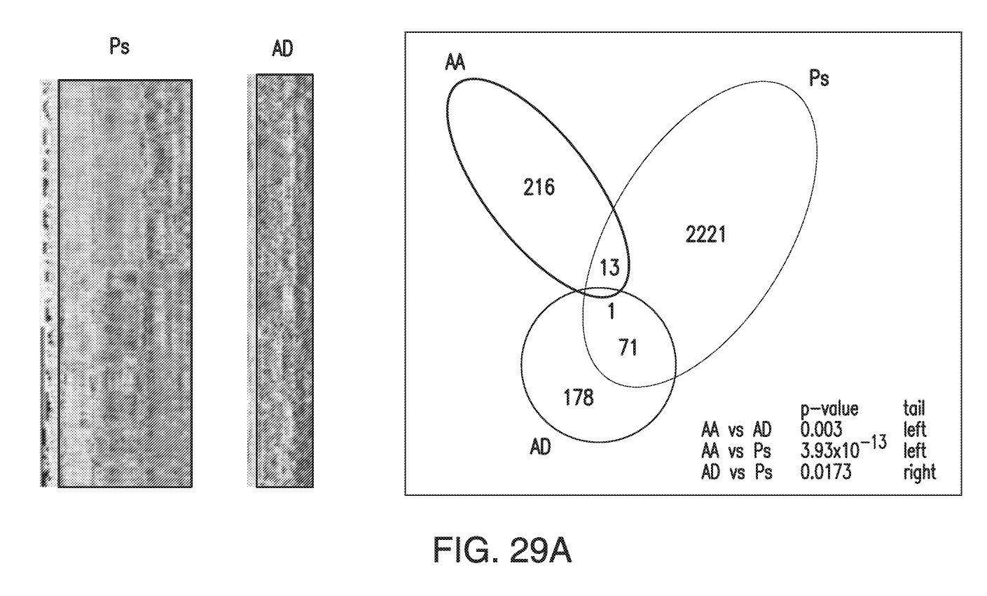

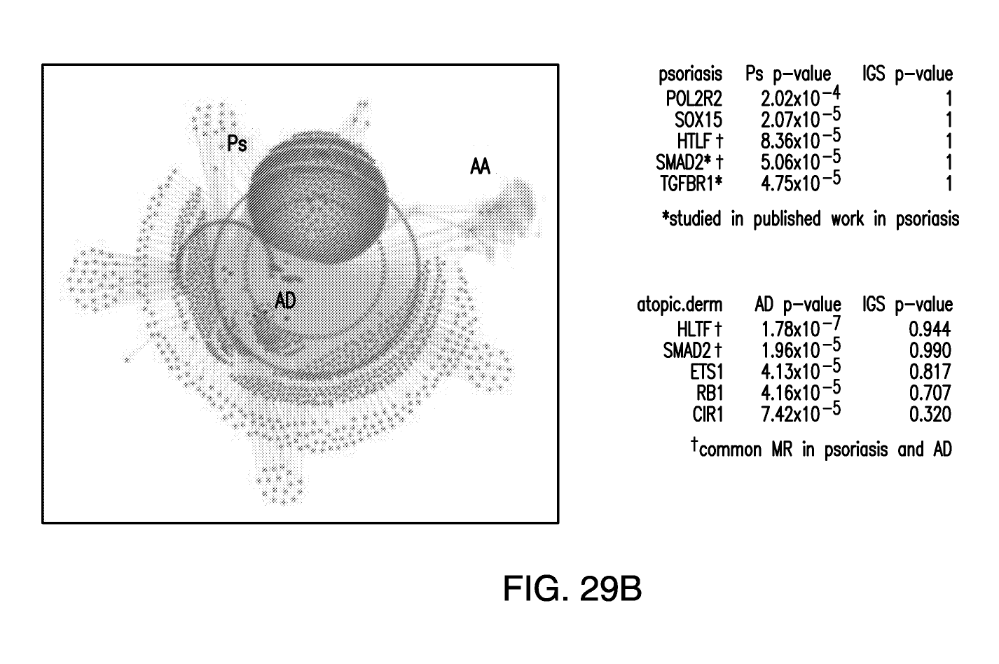

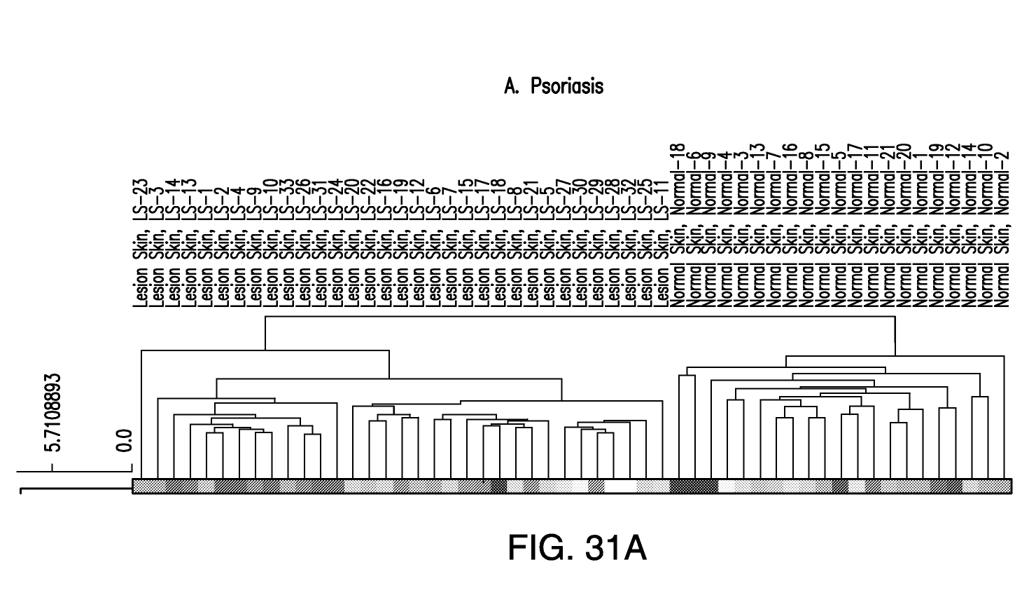

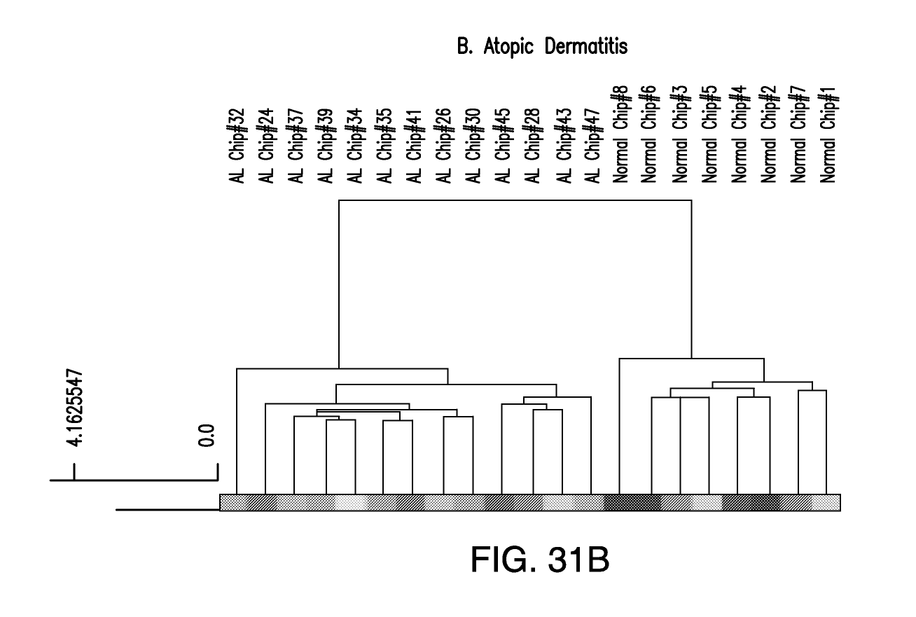

For comparison, and to provide proof-of-concept for the generalizability of the approach, the inventors downloaded publicly available gene expression data sets for atopic dermatitis (AD) and psoriasis (Ps). The inventors generated gene expression signatures for each disease by comparing lesional biopsies to unaffected biopsies, similar to the AA analysis (FIGS. 29A and 31). A direct comparison of the genes within the AA, AD, and Ps signatures revealed statistically significant evidence that the AAGS is distinct from both AD and Ps (p=0.003 and 3.93×10−13, respectively). By contrast, comparison of the AD and Ps signatures to each other revealed statistically significant evidence for shared molecular signatures and, by extension, possible shared molecular pathology (p=0.0173).

These two signatures were applied to the pipeline. FIG. 29B reports the top five MRs identified after the analysis, ranked by their total coverage of the appropriate disease signatures (Ps or AD). Also provided are the ranks of the MRs using the corresponding deconvolved IGS. The results indicate that the key regulatory hubs associated with AA (specifically IKZF1 and DLX4) are unique to AA. Each disease was assigned its own unique list of MRs, but there additionally was overlap of two candidate MRs in AD and Ps: SMAD2 and HLTF. SMAD2 and TGFBR1 are TFs with published evidence of involvement in Ps, and the pipeline was able to identify them with no a priori evidence, using a basic definition of a Ps gene expression signature. These results demonstrate the effectiveness of modeling complex genetic behaviors as regulatory modules to differentiate mechanisms of pathology.

Discussion

Systemic generation and analysis of gene regulatory networks and gene expression data capitalizing on genome-wide profiling has proven to be instrumental in the study of complex diseases. Integrative projects to interrogate functional interactions have recently been leveraged in genome-wide expression signature deconvolution and cross-tissue interactions in diabetes and atherosclerosis. These studies have been invaluable in identifying infiltrating gene signatures, which provide insight into the types of pathogenic immune infiltrates associated with disease. They have also helped identify driver genes from eQTLs and other genomic association tests, similar to the systematic algorithms being developed in cancer and Alzheimer's disease research by providing significant genome-level coverage of regulatory activity and tissue-level gene panels of interacting tissues.

However, particularly in contexts such as AA, little has been done to characterize the modular regulation of discrete pathogenic molecular behaviors within a gene expression profile and how they translate to physiological interactions between tissues of the disease. Modeling physiological traits as genetic programs controlled by MRs provides a uniquely powerful perspective in the study of complex disease. The approach canalizes large gene expression signatures into a relatively few number of selected MRs that subsequently become the T of manipulation via gene therapies or drugs and small molecules.

Here, the inventors extend the application of regulatory networks to interrogate the complex molecular state of a mixed sample of end organ (scalp skin) and infiltrating (immune infiltrates) tissue in AA by comparing regulatory networks of different skin contexts (infiltrated and normal). The inventors establish that in addition to their typical use for identifying the key regulatory hubs governing molecular phenotype switches, these networks can be used to isolate and compartmentalize molecular behaviors that originate from different tissues based on whether or not they are accurately represented in an independent context-specific network. This allows for more precise identification of tissue-specific molecular programs from a mixed sample that contribute to an integrated, interactive physiological behavior such as immune infiltration. Using this pipeline, the inventors were able to reconstruct the MRs mediating infiltration from the skin not only in the context of AA, but the analysis of Ps and AD provides additional candidates for the genetic regulation of inflammatory skin diseases in general, and demonstrates the general applicability of the approach.

Aside from the direct implications in AA pathology, this work provides the proof-of-principle for two key, generalizable notions: (1) a complex interaction between two tissues can be modeled as quantifiable, molecular gene expression modules; and (2) these modules and their regulators can be extracted from expression data, compartmentalized to a tissue, and co-opted to induce the associated interaction in normal cell types. This was evidenced by the ability to recapitulate the AAGS upon ectopic expression of MRs IKZF1 and DLX4 and to subsequently induce enhanced cytotoxicity in non-AA cell lines using normal (non-AA) PBMCs solely via the manipulation of IKZF1 and DLX4 expression within the end organ itself (no genetic manipulation of the PBMCs).

Specifically, the analysis identified MRs that are sufficient to induce interactions with immune cells when expressed solely in scalp skin. Even in a patient-matched context with samples from a healthy, AA-unaffected patient, IKZF1 and DLX4 expression were sufficient to induce aberrant NKG2D-depedent interactions between dermal fibroblasts and PBMCs resulting in cytotoxicity. These interactions were not present in control transfections and they were repeated in two other (nonpatient-matched) cell types, indicating that the expression of IKZF1 or DLX4 is sufficient to induce interactions with normal immune cells irrespective of the specific tissue or host matching. The identification of IKZF1 and DLX4 would have been impossible without the network-based deconvolution, since the significant presence of infiltrating signature in the original AAGS would have prevented any accurate identification of candidate MRs. Instead, network-based deconvolution identified MRs that are capable of inducing specific molecular interactions in any of several molecular contexts that are completely independent of AA itself.

The identification of IKZF1 was unexpected, since IKZF1 is widely studied in the context of T cell differentiation. However, its identification came solely from using a deconvolved AA signature, and not the IGS, using regulatory logic derived from skin. Had the inventors relied on public databases, previous literature, or GO annotations to filter the gene expression data, the inventors would have disregarded and removed IKZF1 entirely due to extensive annotation as a T cell differentiation factor. Instead, by turning to regulatory networks, the inventors were able to identify the possibility that local expression of IKZF1 could have a pathogenic relevance independent of its established role directly in immune cells.

While IKZF1 is well characterized in the context of immune cells, a role for IKZF1 outside of immune cells is not without precedent in the literature. The losses of IKZF1 and DLX4 loci are also associated with oncogenesis in colorectal, lung, and breast cancers, and low-grade squamous intraepithelial lesions. These studies obtain their genomic information directly from tumor masses, indicating that somatic losses of these two loci can contribute to cancer pathophysiology as end organ genomic alterations. The studies into IKZF1 and DLX4 as MRs inducing immune infiltration support these results and raise the possibility that the loss of these loci may contribute to immune evasion in cancer. Further, these observations, and the identification of IKZF1 and DLX4 as MRs of immune infiltration recruitment, provide support that there is a function for IKZF1 outside of its role as a T cell-specific differentiation factor and raises support for the hypothesis that autoimmunity in AA and tumor immune-evasion exist at opposite extremes of normal immune interactions. The loss of the MRs of immune infiltration is associated with cancer, and their overexpression is associated with the onset of autoimmune disease in AA.

The inventors have shown that systems biology and network analysis can be used to model the molecular mechanisms mediating interactions between two distinct tissues, identify the key regulators, and use them to re-create the interactive trait in other contexts. While the output for the validation of these MRs was ultimately induction of cell death, the function of these MRs in the context of autoimmune disease is to induce a molecular profile that ultimately signals to and recruits immune infiltrates. Up to this point, applications of systems biology have mainly been to identify “breakpoints” in cell-autonomous molecular behaviors of cancers. The controlled induction of cross-tissue interactions, particularly those involving the immune system, invites potentially significant avenues for modeling complex genetic traits with regulatory networks that has previously not been feasible. The inventors provide a proof-of-concept framework that can be used to actively compartmentalize molecular behaviors for study even in complex diseases involving interactions between different tissues.

Experimental Procedures

This section contains a description of the less common or unique methods implemented in this study.

ARACNe

To generate a context-specific transcriptional interaction network for scalp skin, the inventors employed the ARACNe algorithm on a set on of 128 microarray experiments independent of the analytic cohorts in this study. These experiments represent platform-matched (Affymetrix U133 2plus) data acquired on whole skin samples from a mixture of normal whole skin biopsies, AA patient biopsies, microdissected dermal papillae, and separated dermis and epidermis samples. These samples collectively provide the heterogeneity required for accurate detection of transcriptional dependencies in the scalp skin. The experiments were pooled and post-processed as described above and a standard ARACNe analysis was performed. The ARACNe software suite is available from the Califano lab web site, http://wiki.c2b2.columbia.edu/califanolab/index.php/Software.

MR Analysis

MRs for a specific gene expression signature were defined as TFs whose direct ARACNe-predicted T (regulon) are statistically enriched in the gene expression signature. Each TF's regulon was tested for enrichment of the AAGS using Fisher's exact test, FDR=0.05. This analysis allows for the ranking and determination of the minimum number of TFs required to specifically cover a gene expression module associated with a physiological trait. http://wiki.c2b2.columbia.edu/califanolab/index.php/Software/MARINA

MR Activity Classifiers

The ARACNe-predicted T of IKZF1 and DLX4 were integrated with the exogenous gene expression studies to identify all genes in the AAGS that could be mapped as T of IKZF1 and DLX4. This was done by intersecting the ARACNe regulons of IKZF1 and DLX4 with the AAGS. The intersection of these two sets was then screened in the expression studies for any genes that responded with at least 25% fold change. This set of genes was used to construct a consensus “meta-activity” for the IKZF1 and DLX4 loci. The rank-normalized change of each gene across the AA patient cohort was integrated into an average as a consensus measure of the relative activity of the parent MR.

These values were subsequently used to define a 2D search space, Math Eq, where X=IKZF1 meta-activity and Y=DLX4 meta-activity, to classify each of the patients in the AA training set. The meta-activity vectors were rank transformed such that the minimum values were bound to the origin of the search space (0,0) and such that activity measures were positive. This transformation has no influence on the results other than projecting the search space into a more intuitive grid for display purposes, in which both axes are bound between [0,n], where n is positive.

Classification in this space was done using a modified nonlinear, soft-margin SVM algorithm. The algorithm is formalized:

$$X = ranksort ( activity \text{?} )$$

$$Y = ranksort ( activity \text{?} )$$

$$define ( A \times B ) \text{:} ∀ a ∈ X , {arg max}_{\text{?}} f ( a , b ) = ( \frac{p ( S \text{?} Q \text{?} ) \times p ( S \text{?} Q \text{?} )}{p ( S \text{?} Q \text{?} ) \times p ( S \text{?} Q \text{?} )} )$$

$$\text{?} \text{indicates text missing or illegible when filed}$$

The algorithm defines a vector set Math Eq, which exists within the search space Math Eq, such that every given pair Math Eq maximizes the likelihood ratio Math Eq. This function is defined such that Math Eq is the next order of disease severity to Math Eq and Math Eq and Math Eq are the quadrants I and III of the grid created by the hyperplanes Math Eq and Math Eq. Samples in the training set are mapped to each grid with known molecular subtypes and the likelihood ratio is computed for the segregation of subtypes defined by Math Eq. The severity ranking used for Math Eq was Normal<Mild<Severe. Each coordinate set in Math Eq therefore defines the points to a nonlinear plane that maximizes the separation between samples of different molecular classes in the IKZF1/DLX4 meta-activity space.

Cytotoxicity Assay

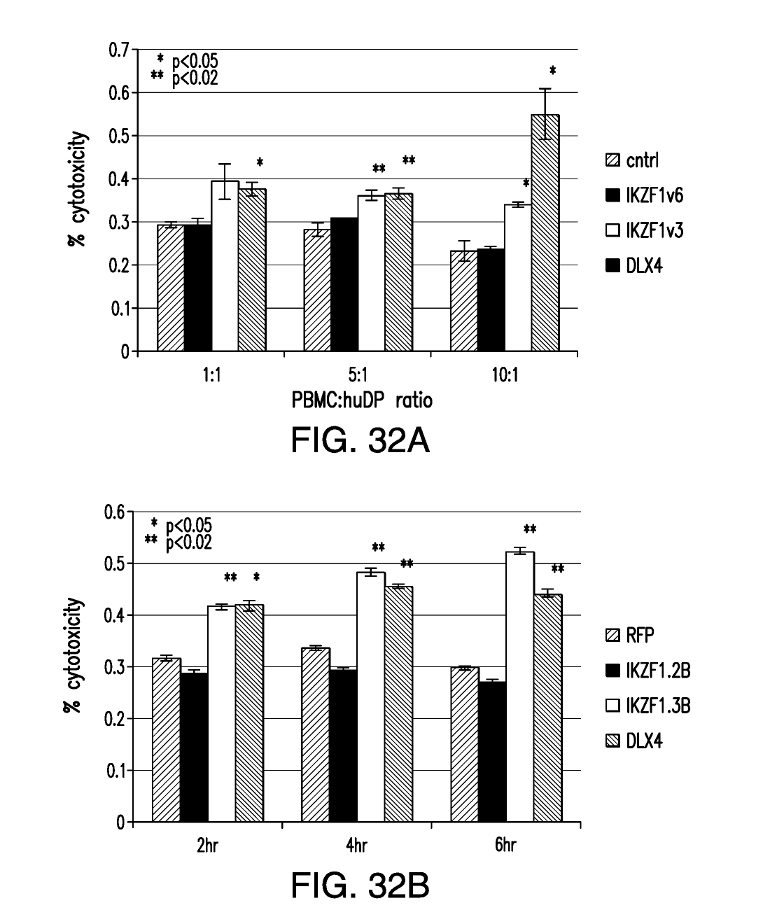

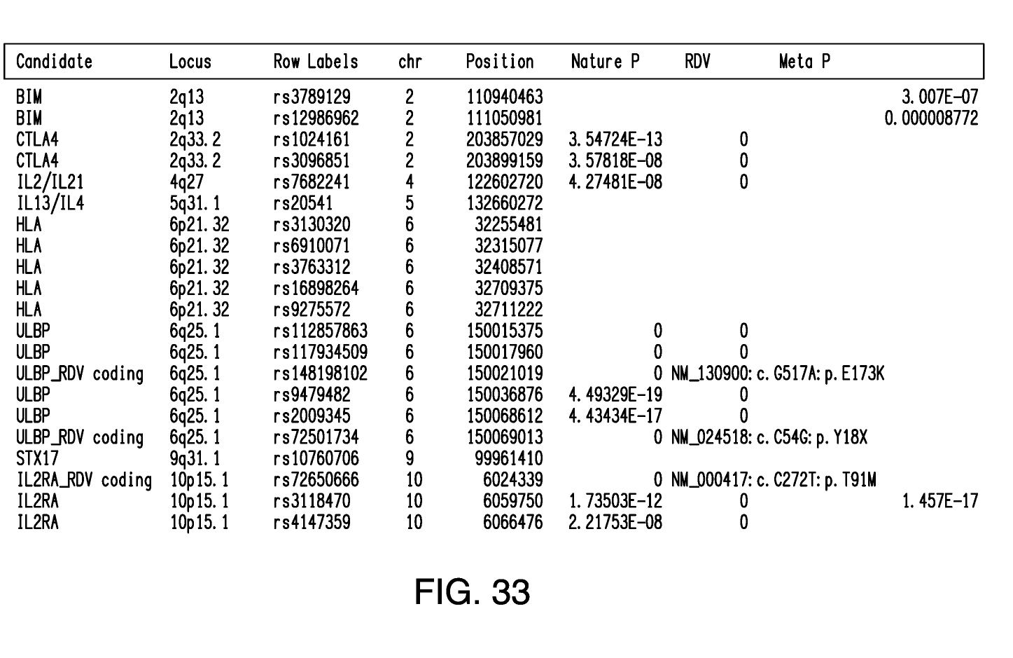

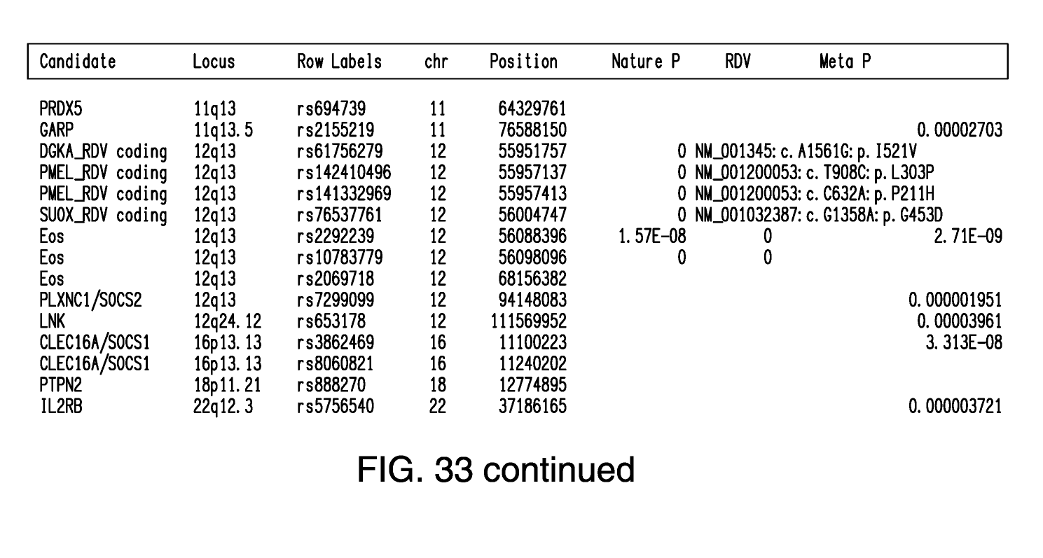

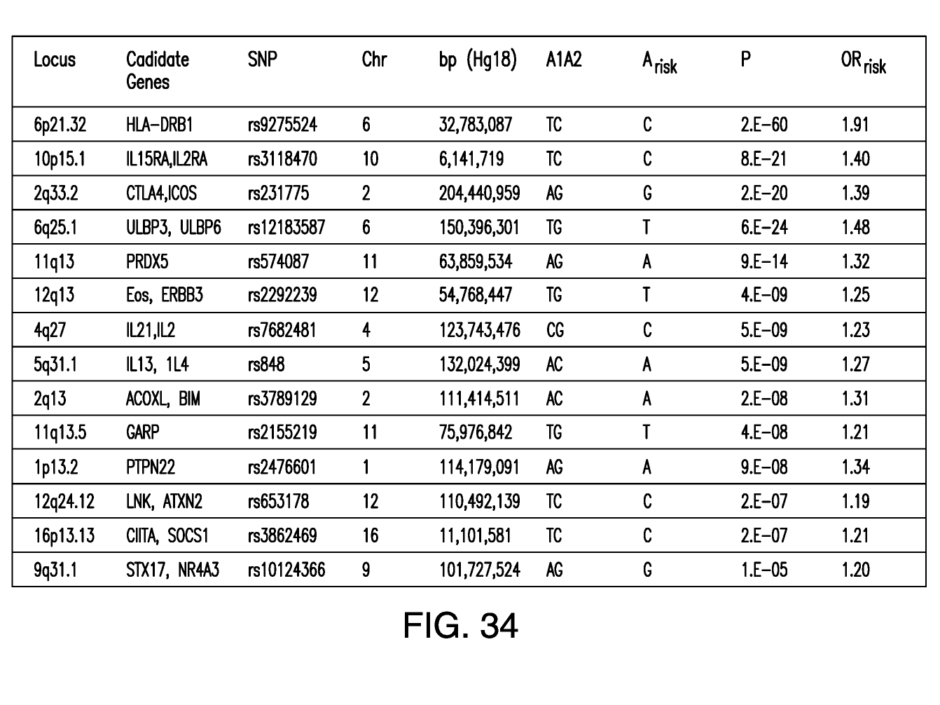

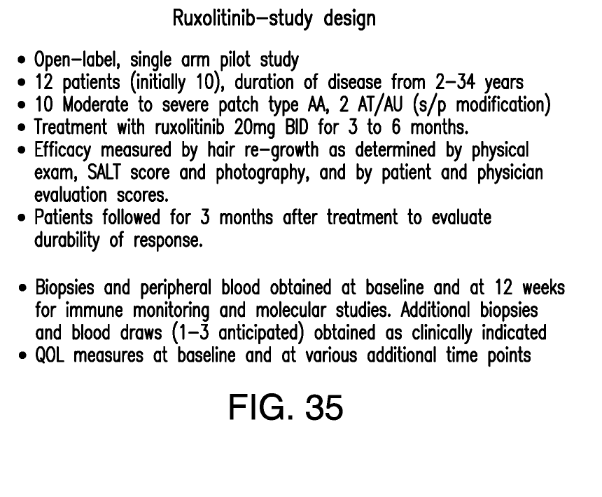

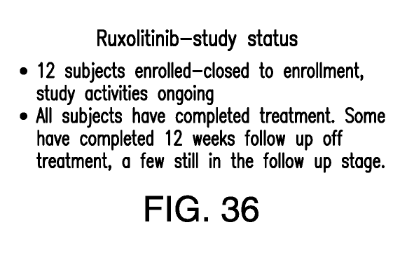

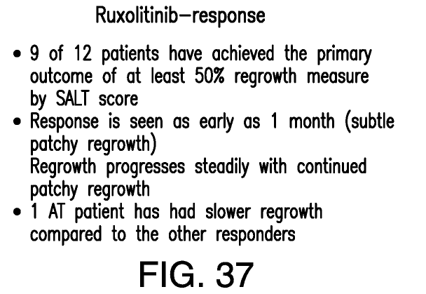

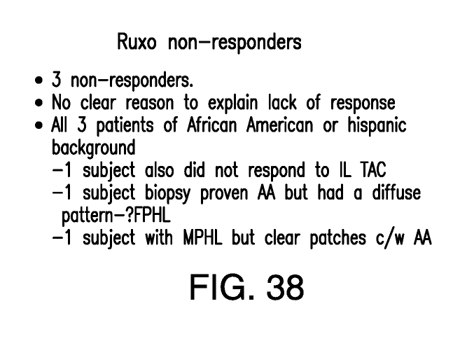

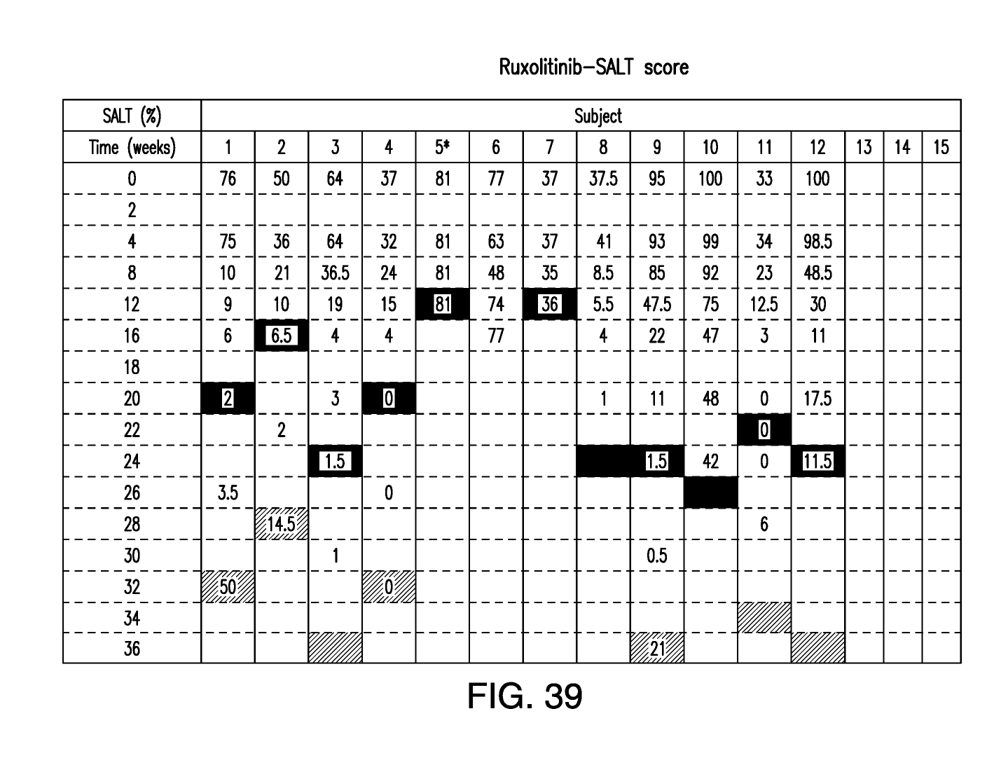

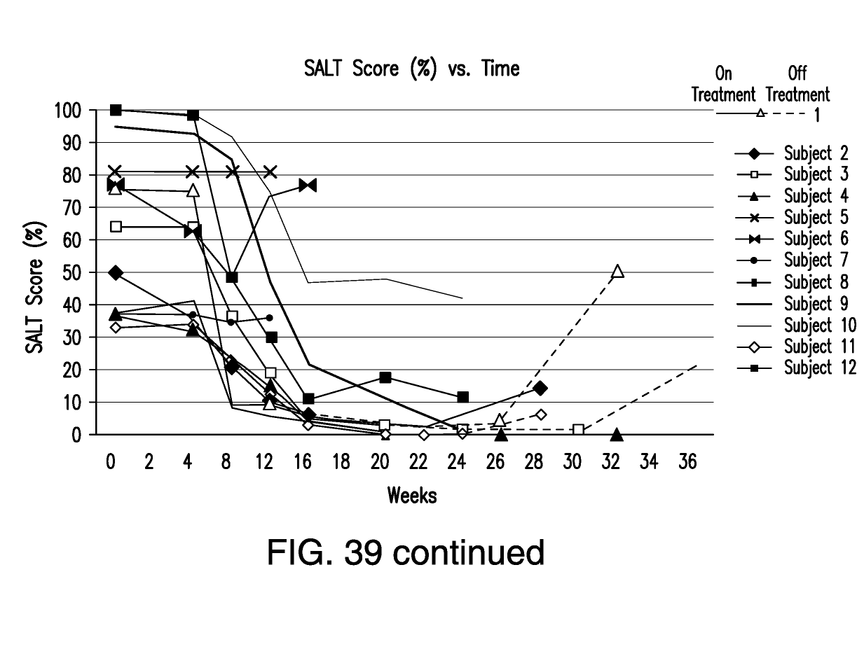

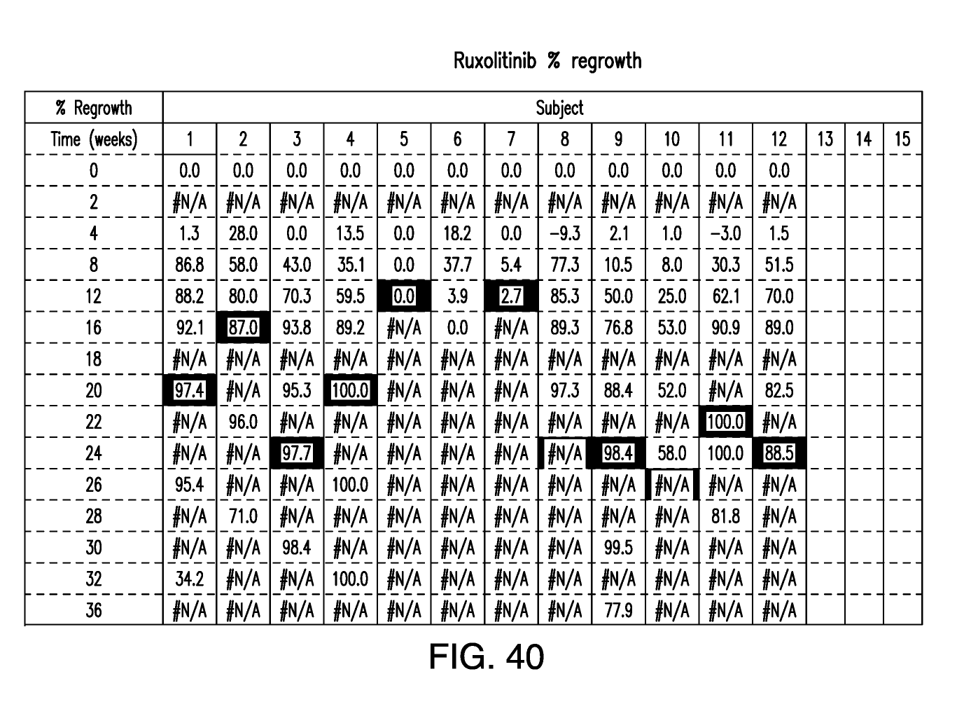

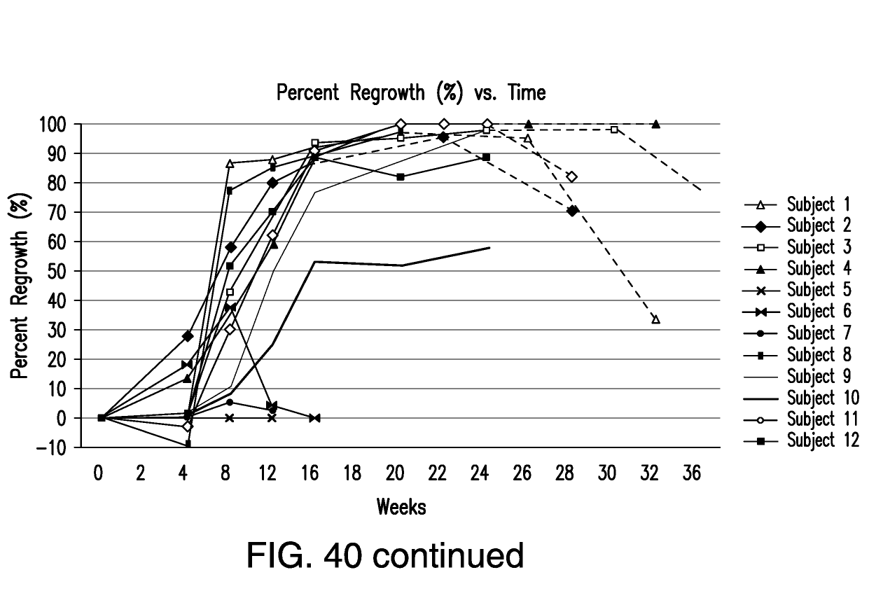

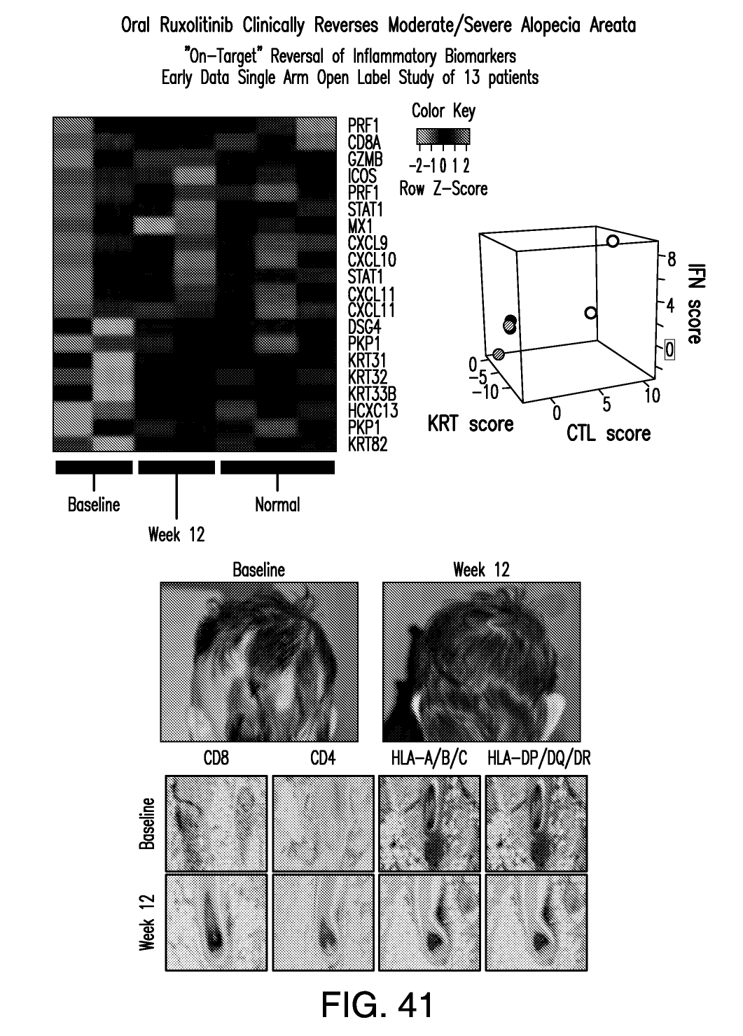

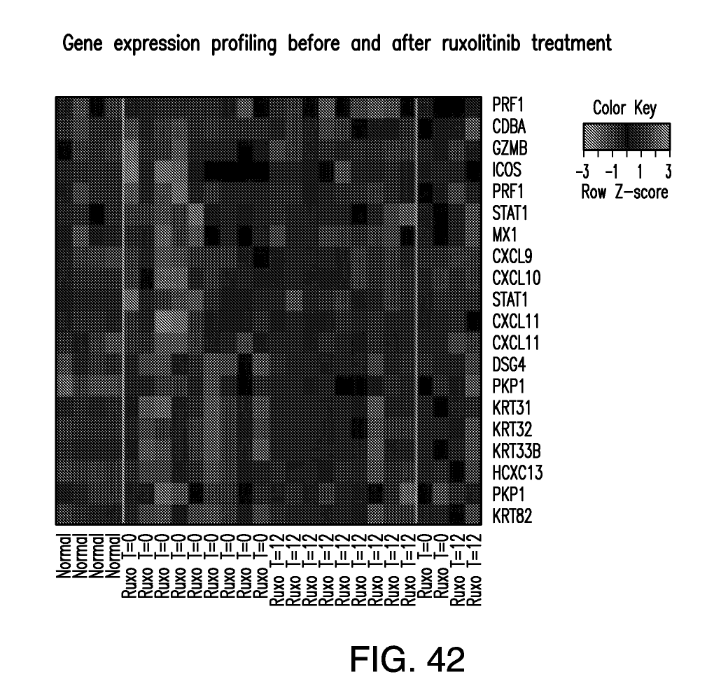

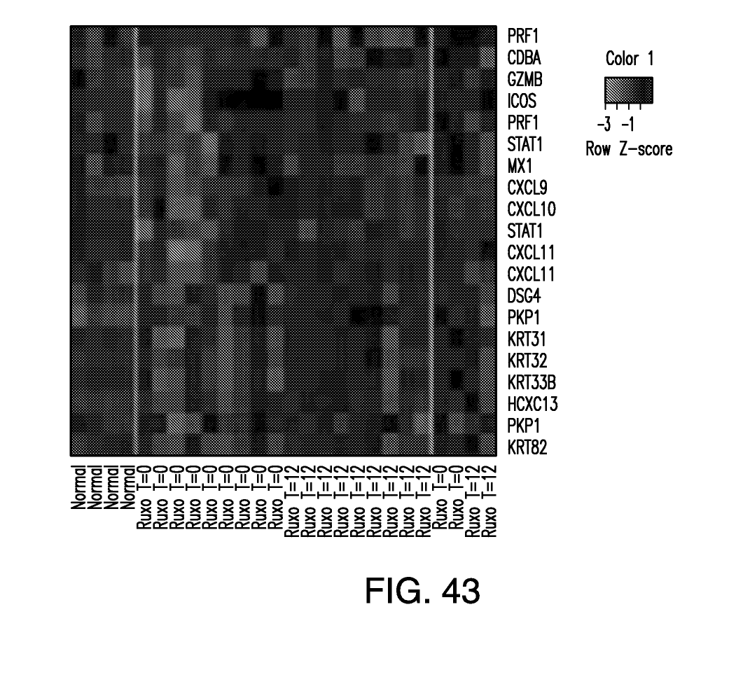

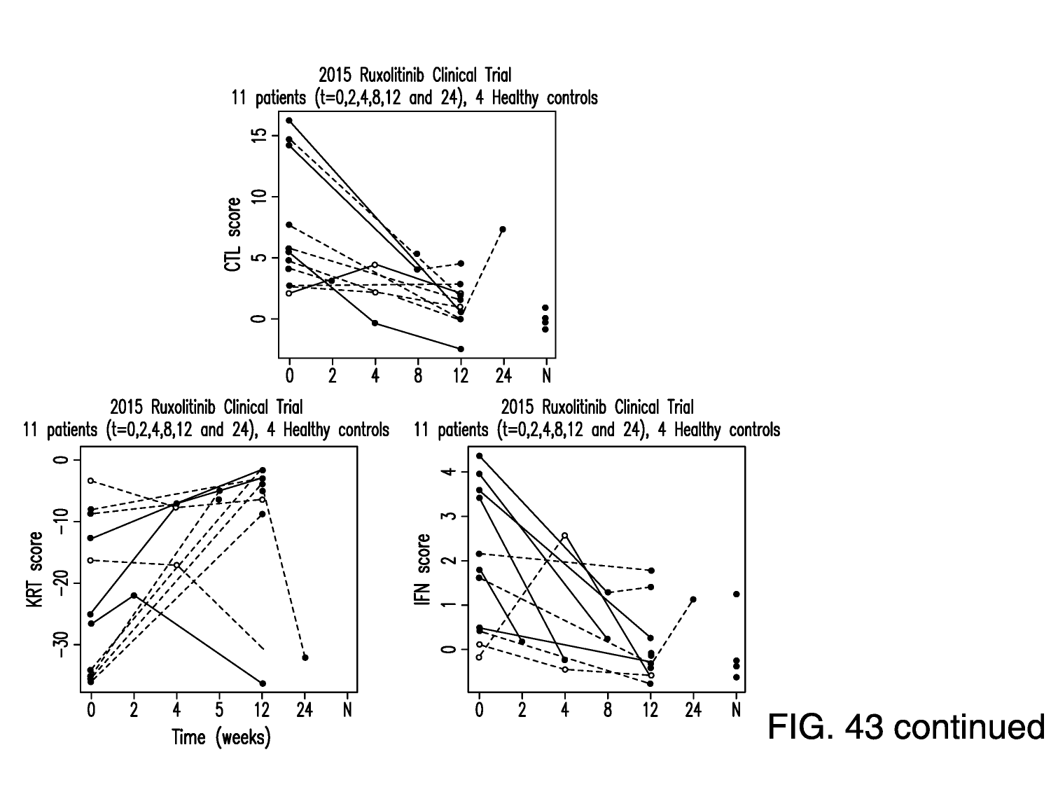

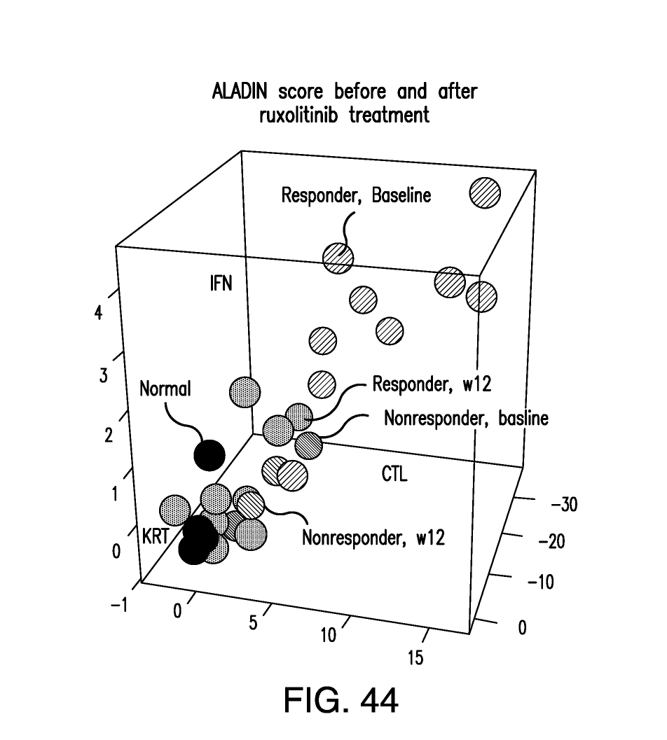

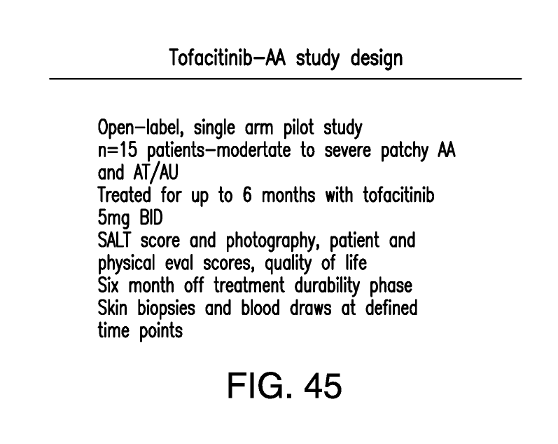

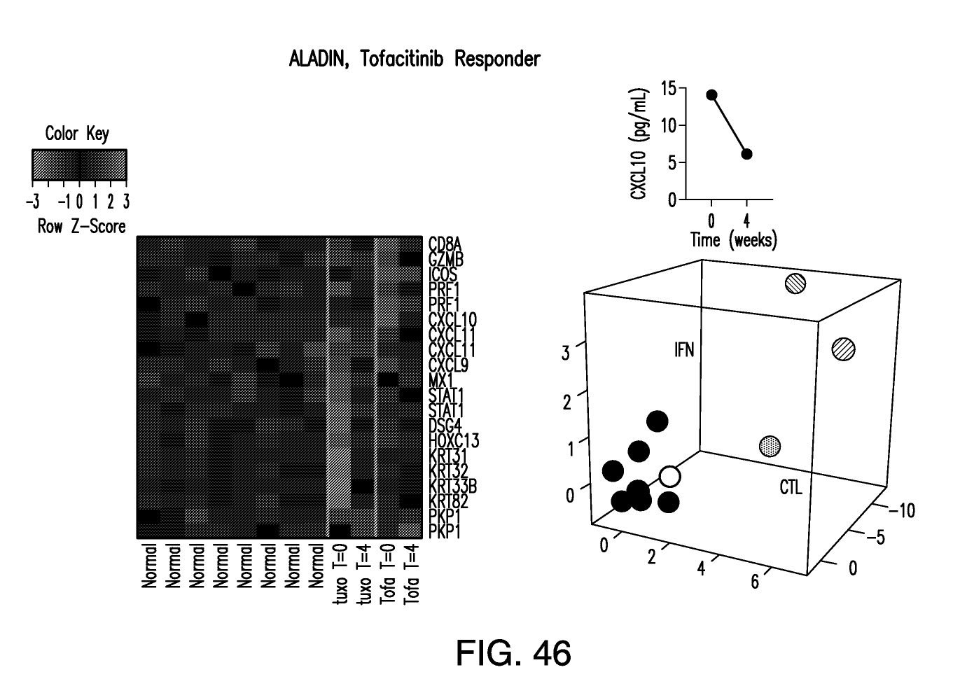

PBMC-dependent cytotoxicity was measured using the CytoTox 96 Nonradioactive Cytotoxicity Assay available through Promega. For the processing of samples and solutions, the inventors followed manufacturer protocols. The optimization for PBMC:T was done as below, but using variable concentrations (1:1, 5:1, and 10:1) (FIG. 32).

Cytotoxicity experiments were set up in 96-well format, with each treatment done in triplicate. Transfections were done 36 hr prior to the experiment. The day of the experiment, HK and huDP cells were trypsinized and diluted with Dulbecco's modified Eagle's medium (DMEM) into working stocks. The T concentration per well was 80,000 cells in 50 μl DMEM, combined with 800,000 PBMCs. The NKG2D inhibitor was the Human NKG2D MAb (clone 149810) from R&D Systems (Cat. MAB139), used at a final concentration of 20 μg/ml. Each transfection was allocated in triplicate according to manufacturer instructions.

Gene Expression Studies

A total of 122 samples from 96 patients were profiled on the Affymetrix U133 2Plus array consisting of 28 AAP patients, 32 AT/AU patients, and 36 unaffected controls. The remaining 26 samples correspond to patient-matched non-lesional biopsies from the AAP cohort. These non-lesional samples were not included in the inference of an initial signature, but used later (below). RNA from these patient biopsies was isolated and processed on the Affymetrix U133 2Plus array. Data post-processing was done via R using MASS normalization with standard packages available through Bioconductor. These data are available at the Gene Expression Omnibus as GSE68801. This dataset was broken into two sets for training and validation.

An initial panel of gene markers was identified by two differential expression analyses comparing (1) AA vs unaffected and (2) lesional vs non-lesional in the training set. A threshold was set for differential expression at p<0.05 and a fold change>25%. This relatively lax threshold was implemented because the network analyses are based on consensus. The analysis is not primarily concerned with candidate ranks, but instead relies on having enough molecular information to infer TF activity. This approach is also necessarily more robust to noise that could be introduced by a more relaxed threshold, since the addition of noise would be applied across the entire dataset and normalized out of the consensus by both ARACNe and master regulator analysis (see below). All X- and Y-linked genes were additionally removed to remove any possible gender bias in the ranking and clustering of differentially expressed genes.

Gene Set Enrichment Analysis

GSEA is a method for measuring nonparametric statistical enrichment in the differential expression of a defined panel of genes. A default differential expression analysis between experimental and control cohorts done, and genes are rank-sorted by differential expression with no threshold (all genes included). This can be done according to any user-specified criteria (fold-change, p-value, etc).

This enrichment score is then compared to an empirically generated null distribution by shuffling sample labels, i.e., by randomizing case and control samples and repeating the analysis. This is repeated over 1000 iterations to generated a null distribution of Enrichment Scores, which the observed score can be compared against to generate a p-value.

Cloning

Each primer pair provided below was used in PCR reactions with the Accuprime Taq PCR mixes according to manufacturer protocols on cDNAs derived from HEK293T cells. cDNAs were generated from cultured cells using the SuperScript First-Strand Synthesis System from Invitrogen. PCR products were run out by gel electrophoresis, and any isoforms present were separately excised using the Qiagen Gel Extraction Kit.

mRNA fidelity was verified via sequencing from Genewiz, and correct sequences were digested with the appropriate enzymes (SPEI and ASCI) from New England Biosystems in SmartCut buffer for 2 hours. The pLOC-RFP vector was digested in parallel, and the cut backbone was excised by gel extraction. After purification of the backbone and inserts, each insert was ligated into the cut pLOC vector using the RapidLigation Kit from Roche, according to manufacturer protocols and transformed into DH5α cells for amplification.

Successful transformations were validated for sequence fidelity via colony PCR and sequencing (Genewiz). Correct constructs were amplified and purified by Maxiprep (Qiagen) for experiments.

Primers used to clone genes for insertion into the pLOC vector are provided below in the following format, 5′ to 3′: spacer-enzyme-mRNAsequence.

IKZF1.1
Forward
GGC-ACTAGT-ATGGATGCTGATGAGGGTCAA
Reverse
ATT-GGCGCGCC-TTAGCTCATGTGGAAGCGGT
IKZF1.2
Forward
GGC-ACTAGT-ATGGATGCTGATGAGGGTCAAG
Reverse
ATT-GGCGCGCC-TTAGCTCATGTGGAAGCGGT
(identical to 1.1)
DLX4
Forward
GGC-ACTAGT-ATGAAACTGTCCGTCCTACCCC
Reverse
ATT-GGCGCGCC-TCATTCACACGCTGGGGCTGG

Cell Culture and Transfections

Both huDP and HK cells were kept in standard conditions for growth: DMEM 10% FBS at 37 C and 5% CO2. huDP cells are cultured primary human dermal papillae that were microdissected from human skin samples. For the experiments in this work, only huDP and HK cells with a passage number <6 were used.

Cells were transformed with pLOC expression constructs using the JetPRIME transfection reagent according to manufacturer protocols. Transfections were allowed to carry overnight using a 2:1 concentration of reagent (ul) to DNA (ug).

Microarrays of MR Rescue

Transfections of IKZF1 and DLX4 into HK and huDP cells were carried out as described above in cells cultured in 10 cm plates. 36 hours post-transfection these cells were harvested in PBS with a cell scraped, then lysed and processed for purified RNA using the RNeasy kit from Qiagen following manufacturer protocols. RNA quality control was done using a spectrometer and submitted for processing on the Affymetrix human U133 2Plus array by the Columbia facility (Pathology Department). Array data was again normalized and processed using MASS normalization through the Bioconductor package in R.

qPCRs

Quantitative PCR reactions were performed on cDNAs extracted from an independent cohort of eight primary lesional biopsies (one was found to be degraded and was excluded from the study), four unaffected controls, and five pairs of patient-matched lesional and non-lesional samples. Reaction mixes using SYBR Green were made in 25 ul volumes according to manufacturer protocols and analyzed on a 7300 series Real Time PCR Machine from Applied Biosystems. Primers for each gene are provided at the end of this section.

All samples were tested in technical triplicates in stamp-plate format (each replicate was performed on one plate, with all samples and controls prepared at once, repeated three times). Data from these replicates was analyzed via the δ δ CT method, normalizing all experimental series to the average normalized values of the control tissues. The SEM was derived across the comparisons using standard statistical error propagation.

Primers for assaying transcripts by qPCR are provided below, 5′ to 3′. The primers for full-length amplification of DLX4 were used because the transcript is ˜300 bp (the optimal transcript length for the provided protocol is 200-300 bp).

IKZF1
Forward
ACTCCGTTGGTAAACCTCAC
Reverse
CTGATCCTATCTTGCACAGGTC
DLX4
*same as cloning primers*
ACTB
Forward
GAAGGATTCCTATGTGGGCGAC
Reverse
GGGTCATCTTCTCGCGGTTG

Isolating fresh Peripheral Blood Mononuclear Cells

Fresh PBMCs were isolated from whole blood draws the evening before the intended cytotoxicity assays. PBMCs were separated from whole blood using the Histopaque-1077 reagent (Ficoll) by diluting 8-ml aliquots of whole blood in sterile PBS 1:1, and layering that solution over Ficoll at a final volumetric ratio of 2:1. This solution was centrifuged at 1200 rpm for 45 minutes. The monocyte-bearing interface layer was isolated, diluted in 5× volumes of sterile PBS and centrifuged again for 15 minutes at 1500 rpm. Supernatant was discarded, and the pellet was resuspended in 3 ml of DMEM 10% FBS. Cell count was performed with a hemocytometer and the solution was diluted to a final concentration of 1×106 cells per ml with DMEM 10% FBS. This was stored overnight at 37 C and 5% CO2 for the experiments next-morning.

In addition to the various embodiments depicted and claimed, the disclosed subject matter is also directed to other embodiments having other combinations of the features disclosed and claimed herein. As such, the particular features presented herein can be combined with each other in other manners within the scope of the disclosed subject matter such that the disclosed subject matter includes any suitable combination of the features disclosed herein. The foregoing description of specific embodiments of the disclosed subject matter has been presented for purposes of illustration and description. It is not intended to be exhaustive or to limit the disclosed subject matter to those embodiments disclosed.

Various publications, patents and patent applications, and protocols are cited herein, the contents of which are hereby incorporated by reference in their entireties.

1. A method of treating Alopecia Areata (AA) in a subject comprising:
(a) identifying the AA disease severity in said subject by detecting a biomarker indicative of said disease severity; and
(b) administering a therapeutic intervention to said subject appropriate to the identified disease severity.

2. A method of treating Alopecia Areata (AA) in a subject comprising:
(a) identifying the propensity of a subject having AA to respond to JAK inhibitor treatment by detecting a biomarker indicative of said propensity; and
(b) administering a JAK inhibitor to said subject if the identified biomarker indicates a propensity that the subject will respond to said inhibitor.

3. A method of treating Alopecia Areata (AA) in a subject comprising:
(a) administering a JAK inhibitor to the subject; and
(b) detecting a biomarker indicative of responsiveness to JAK inhibitor treatment; and
(c) thereafter tailoring administration of the JAK inhibitor based on the responsiveness by either (1) continuing administration of the JAK inhibitor, (2) altering administration of the JAK inhibitor, or (3) discontinuing administration of the JAK inhibitor.

4. The method of claim 1, wherein said detecting of the biomarker is performed on a sample obtained from the subject and the sample is selected from the group consisting of skin, blood, serum, plasma, urine, saliva, sputum, mucus, semen, amniotic fluid, mouth wash and bronchial lavage fluid.

5-8. (canceled)

9. The method of claim 4, wherein the biomarker is a gene expression signature comprising gene expression information of one or more of the following groups of genes: KRT-associated genes; CTL-associated genes; and IFN-associated genes.

10. The method of claim 9, wherein the KRT-associated genes comprise DSG4, HOXC31, KRT31, KRT32, KRT33B, KRT82, PKP1 and PKP2.

11. The method of claim 9, wherein the CTL-associated genes comprise CD8A, GZMB, ICOS and PRF1.

12. The method of claim 9, wherein the IFN-associated genes comprise CXCL9, CXCL10, CXCL11, STAT1 and MX1.

13. The method of claim 4, wherein the biomarker is a gene expression signature that is an Alopecia Areata Disease Activity Index (ALADIN).

14. (canceled)

15. The method of claim 4, wherein the biomarker is a gene expression signature that is IKZF1, DLX4 or a combination thereof.

16-26. (canceled)

27. The method of claim 2, wherein said detecting of the biomarker is performed on a sample obtained from the subject and the sample is selected from the group consisting of skin, blood, serum, plasma, urine, saliva, sputum, mucus, semen, amniotic fluid, mouth wash and bronchial lavage fluid.

28. The method of claim 27, wherein the biomarker is a gene expression signature comprising gene expression information of one or more of the following groups of genaes: KRT-associated genes; CTL-associated genes; and IFN-associated genes.

29. The method of claim 28, wherein the KRT-associated genes comprise DSG4, HOXC31, KRT31, KRT32, KRT33B, KRT82, PKP1 and PKP2.

30. The method of claim 28, wherein the CTL-associated genes comprise CD8A, GZMB, ICOS and PRF1.

31. The method of claim 28, wherein the IFN-associated genes comprise CXCL9, CXCL10, CXCL11, STAT1 and MX1.

32. The method of claim 27, wherein the biomarker is a gene expression signature that is an Alopecia Areata Disease Activity Index (ALADIN).

33. The method of claim 27, wherein the biomarker is a gene expression signature that is IKZF1, DLX4 or a combination thereof.

34. The method of claim 3, wherein said detecting of the biomarker is performed on a sample obtained from the subject and the sample is selected from the group consisting of skin, blood, serum, plasma, urine, saliva, sputum, mucus, semen, amniotic fluid, mouth wash and bronchial lavage fluid.

35. The method of claim 34, wherein the biomarker is a gene expression signature comprising gene expression information of one or more of the following groups of genes: KRT-associated genes; CTL-associated genes; and IFN-associated genes.

36. The method of claim 35, wherein the KRT-associated genes comprise DSG4, HOXC31, KRT31, KRT32, KRT33B, KRT82, PKP1 and PKP2.

37. The method of claim 35, wherein the CTL-associated genes comprise CD8A, GZMB, ICOS and PRF1.

38. The method of claim 35, wherein the IFN-associated genes comprise CXCL9, CXCL10, CXCL11, STAT1 and MX1.

39. The method of claim 34, wherein the biomarker is a gene expression signature that is an Alopecia Areata Disease Activity Index (ALADIN).

40. The method of claim 34, wherein the biomarker is a gene expression signature that is IKZF1, DLX4 or a combination thereof.


SEQUENCE LISTING (9 sequences)

SEQ ID NO: 1 - DNA, 30 bp, Artificial Sequence
  Description of Artificial Sequence Synthetic primer
ggcactagtatggatgctgatgagggtcaa

SEQ ID NO: 2 - DNA, 31 bp, Artificial Sequence
  Description of Artificial Sequence Synthetic primer
attggcgcgccttagctcatgtggaagcggt

SEQ ID NO: 3 - DNA, 31 bp, Artificial Sequence
  Description of Artificial Sequence Synthetic primer
ggcactagtatggatgctgatgagggtcaag

SEQ ID NO: 4 - DNA, 31 bp, Artificial Sequence
  Description of Artificial Sequence Synthetic primer
ggcactagtatgaaactgtccgtcctacccc

SEQ ID NO: 5 - DNA, 32 bp, Artificial Sequence
  Description of Artificial Sequence Synthetic primer
attggcgcgcctcattcacacgctggggctgg

SEQ ID NO: 6 - DNA, 20 bp, Artificial Sequence
  Description of Artificial Sequence Synthetic primer
actccgttggtaaacctcac

SEQ ID NO: 7 - DNA, 22 bp, Artificial Sequence
  Description of Artificial Sequence Synthetic primer
ctgatcctatcttgcacaggtc

SEQ ID NO: 8 - DNA, 22 bp, Artificial Sequence
  Description of Artificial Sequence Synthetic primer
gaaggattcctatgtgggcgac

SEQ ID NO: 9 - DNA, 20 bp, Artificial Sequence
  Description of Artificial Sequence Synthetic primer
gggtcatcttctcgcggttg